(19) United States
(12) Patent Application Publication
(10) Pub. No.: US 2018/0330049 A1
(43) Pub. Date: Nov. 15, 2018

(54) METHODS FOR CLASSIFICATION OF GLIOMA

(71) Applicants: THE TRUSTEES OF COLUMBIA UNIVERSITY IN THE CITY OF NEW YORK, NEW YORK, NY (US); University of São Paolo, São Paulo (BR); University of Texas MD Anderson Cancer Center, Houston, TX (US)

(72) Inventors: Antonio Iavarone, New York, NY (US); Houtan Noushmehr, São Paulo (BR); Roel GW Verhaak, Houston, TX (US)

(21) Appl. No.: 16/035,392

(22) Filed: Jul. 13, 2018

Related U.S. Application Data
(63) Continuation of application No. PCT/US2017/014561, filed on Jul. 23, 2017.
(60) Provisional application No. 62/286,117, filed on Jan. 22, 2016.

(51) Int. Cl. G06F 19/20; G06F 19/22

(52) U.S. Cl. CPC G06F 19/20; G06F 19/22

(57) ABSTRACT

The present disclosure provides a method of classifying a glioma in a patient by identifying with respect to the glioma, isocitrate dehydrogenase genes (IDH) mutation status, DNA methylation cluster, RNA cluster, telomere length, telomere maintenance, and at least one biomarker, and based in the identifications, classifying the glioma as IDH mutant/G-CIMP low glioma type, IDH mutant/G-CIMP high glioma type, IDH mutant/Codel glioma type, DH wild type/Classic like glioma type, IDH wild type/Mesenchymal-like glioma type, IDH wild type/LGm6-GBM glioma type, or PA-like glioma type.

### PRIORITY CLAIM

The present application is a continuation of International Patent Application PCT/US17/014561 filed Jan. 23 2017; which claims priority to U.S. Provisional Patent Application Ser. No. 62/286,117, filed Jan. 22, 2016, titled “Methods for Accurate Stratification of Patients With Glioma,” the contents of which are incorporated by reference herein in their entirety.

### TECHNICAL FIELD

The present disclosure relates to methods of accurately classifying patients with glioma, and to methods of treating patients with glioma based on the classification.

### BACKGROUND

Diffuse gliomas represent 80% of malignant brain tumors. Adult diffuse gliomas are classified and graded according to histological criteria (oligodendroglioma, oligoastrocytoma, astrocytoma, and glioblastoma; grade II to IV). Although histopathologic classification is well established and is the basis of the World Health Organization (WHO) classification of CNS tumors, it suffers from high intra- and inter-observer variability, particularly among grade II-III tumors. Recent molecular characterization studies have benefited from the availability of the datasets generated by The Cancer Genome Atlas (TCGA) and have related genetic, gene expression, and DNA methylation signatures with prognosis.

For example, mutations in the isocitrate dehydrogenase genes 1 and 2 (IDH1/IDH2) define a distinct subset of glioblastoma (GBM) with a hypermethylation phenotype (G-CIMP) with favorable outcome. Conversely, the absence of IDH mutations in low grade glioma (LGG) marks a distinct IDH-wild-type subgroup characterized by poor, GBM-like prognosis. Accordingly, it has been proposed to classify glioma into IDH wild-type cases, IDH mutant group additionally carrying codeletion of chromosome arm 1p and 19q (IDH mutant-codel) and samples with euploid 1p/19q (IDH mutant-non-codel), regardless of grade and histology. Mutation of the telomerase reverse transcriptase (TERT) promoter, which has been reported with high frequency across glioma, may be an additional defining feature. Current analyses have not clarified the relationships between LGGs and GBMs that share common genetic hallmarks like IDH mutation or TERT promoter mutation status.

### SUMMARY

The present disclosure, according to a non-limiting embodiment, provides a method of classifying a glioma in a patient by identifying with respect to the glioma, isocitrate dehydrogenase genes (IDH) mutation status, DNA methylation cluster, RNA cluster, telomere length, telomere maintenance, and at least one biomarker, and based in the identifications, classifying the glioma as IDH mutant/G-CIMP low glioma type, IDH mutant/G-CIMP high glioma type, IDH mutant/Codel glioma type, DH wild type/Classic like glioma type, IDH wild type/Mesenchymal-like glioma type, IDH wild type/LGm6-GBM glioma type, or PA-like glioma type.

The present disclosure, according to certain non-limiting embodiments, further provides the following additional features, which may be combined with one another unless clearly mutually exclusive:
i) IDH mutation status is based on a mutation in and IDH1 or IDH2 gene, or the presence of a wild type version of both genes;
ii) the DNA methylation cluster is LGm1, LGm2, LGm3, LGm4, LGm5, or LGm6
iii) the RNA cluster is LGr1/2, LGr3, or LGr4;
iv) telomere length is identified as elongated, shortened, or stable.
v) the telomere maintenance is identified based on the presence of mutant to wild type Alpha-thalassemia X-linked (ATRX);
vi) the teolmere maintenance is identified based on the upregulation or normal expression of telomerase reverse transcriptase (TERT);
vii) the biomarker includes upregulation of Epidermal Growth Factor Receptor (EGFR);
viii) the biomarker includes a mutation in Tumor protein p53;
ix) the biomarker includes an IDH mutant-codel;
x) the biomarker includes a chromosome 7 (chr7) amplification coupled with a chromosome 10 (chr10) deletion;
xi) the biomarker includes a Cyclin-dependent kinase 4 (CDK4) amplification coupled with a Cyclin Dependent Kinase Inhibitor 2A (CDKN2A) deletion;
xii) the biomarker includes a chromosome 19 (chr19) amplification coupled with a chromosome 20 (chr20) amplification;
xiii) the biomarker includes a B-Raf gene (BRAF) mutation coupled with a Neurofibromin 1 (NF1) mutation;
xiv) the IDH mutant/G-CIMP low glioma type exhibits an IDH mutation, the LGm1 DNA methylation cluster, the LGr3, RNA cluster, elongated telomere length, an ATRX mutation, a TP53 mutation, and a CDK4 amplification coupled with a CDKN2A deletion;
xv) the IDH mutant/G-CIMP high glioma type exhibits an IDH mutation, the LGm2 DNA methylation cluster, the LGr3 RNA cluster, elongated telomere length, an ATRX mutation, and a TP53 mutation;
xvi) the IDH mutant/Codel glioma type exhibits an IDH mutation, the LGm3 DNA methylation cluster, the LGr1/2 RNA cluster, shortened telomere length, upregulation of TERT, and an IDH mutant-codel;
xvii) the IDH wild type/Classic like glioma type exhibits wild type IDH, the LGm4 DNA methylation cluster, the LGr4 RNA cluster, shortened telomere length, upregulation of TERT, amplified EGFR, amplified chr 7 coupled with a chr 10 deletion, and amplified chr 19 coupled with amplified chr 20;
xviii) the IDH wild type/Mesenchymal-like glioma type exhibits wild type IDH, the LGm5 DNA methylatin cluster, the LGr4 RNA cluster, shortened telomere length, amplified EGFR, and amplified chr 7 coupled with a chr 10 deletion;
xix) the IDH wild type/LGm6-GBM glioma type exhibits wild type IDH, the LGm6 DNA methylation cluster, the LGr4 RNA cluster, stable telomer length, amplified EGFR, a CDK4 amplification coupled with a CDKN2A deletion, and a BRAF mutation coupled with a NF1 mutation;
xx) the PA-like glioma type exhibits wild type IDH, the LGm6 DNA methylation cluster, the LGr4 RNA cluster, stable telomer length, and a BRAF mutation coupled with a NF1 mutation.

According to additional non-limiting embodiments, the present disclosure also provides a method of treating a patient with glioma, by classifying the glioma according to any above and administering a cancer therapy or therapeutically effective amount of a pharmaceutical composition to the patient based on the glioma type.

According to further non-limiting embodiments, the present disclosure also provides a system for classifying a glioma. The system includes a memory and a processor able to receive the identifications from any of the above methods and execute steps to classify the glioma according to any of the above methods.

According to additional non-limiting embodiments, the present disclosure also provides a kit for performing the method of any of the above claims or for use with any of the above systems. The kit includes at least one reagent or other material.

### BRIEF DESCRIPTION OF THE DRAWINGS

For a more complete understanding of the present disclosure and its features and advantages, reference is now made to the following description, taken in conjunction with the accompanying drawings in which:

FIG. 1A is a heatmap of relative tumor/normal telomere lengths of 119 gliomas, grouped by TERT promoter (TERTp) and Alpha-thalassemia X-linked (ATRX) mutation status;

FIG. 1B is a graph of telomere length versus age (measured in years at diagnosis) in blood normal control samples (n=137);

FIG. 1C is a graph of quantitative telomere length estimates of tumors and blood normal, grouped by TERTp mutant (n=67, 56%), ATRX mutant (n=40, 33%), and double negative (n=13, 11%) status. ***=p<0.0001; **=p<0.001;

FIG. 2A is heatmap of DNA methylation data in which columns represent 932 The Cancer Genome Atlas (TCGA) glioma samples grouped according to unsupervised cluster analysis and rows represent DNA methylation probes sorted by hierarchical clustering; non-neoplastic samples are represented on the left of the heatmap (n=77);

FIG. 2B is a heatmap of RNA sequencing data in which unsupervised clustering analysis for 667 TCGA glioma samples profiled using RNA sequencing are plotted in the heatmap using 2,275 most variant genes;

FIG. 2C is a tumor map based on mRNA expression and DNA methylation data in which each data point is a TCGA sample colored coded according to their identified status;

FIG. 3A is a heatmap of probes differentially methylated between the two IDH mutant-non-codel DNA methylation clusters to allow the identification of a low-methylation subgroup named G-CIMP-low; non-tumor brain samples (n=12) are represented on the left of the heatmap;

FIG. 3B is a heatmap of genes differentially expressed between the two IDH mutant-non-codel DNA methylation clusters;

FIG. 3C is a set of Kaplan-Meier survival curves of IDH mutant methylation subtypes; ticks represent censored values;

FIG. 3D is a map of the distribution of genomic alterations in genes frequently altered in IDH mutant gliomas;

FIG. 3E is a map of the genomic distribution of 633 CpG probes differentially demethylated between co-clustered G-CIMP-low and G-CIMP-high; CpG probes are grouped by University of California Santa Cruz (UCSC) genome browser-defined CpG Islands, shores flanking CpG island ±2 kb and open seas (regions not in CpG islands or shores);

FIG. 3F is a DNA methylation heatmap of TCGA glioma samples ordered per FIG. 2A with the epigenetically regulated (EReg) gene signatures defined for G-CIMP-low, G-CIMP-high, and Codel subtypes; the mean RNA sequencing counts for each gene matched to the promoter of the identified cgID across each cluster are plotted to the right;

FIG. 3G is a heatmap of the validation set classified using the Random Forest method applying the 1,300 probes defined in FIG. 2A;

FIG. 3H is a heatmap of probes differentially methylated between G-CIMP-low and G-CIMP-high in longitudinally matched tumor samples;

FIG. 4A is a set of Kaplan-Meier survival curves for IDH-wild-type glioma subtypes; ticks represent censorship;

FIG. 4B is a graph of the distribution of previous published DNA methylation subtypes in the validation set, across the TCGA IDH-wild-type-specific DNA methylation clusters;

FIG. 4C is a map of the distribution of genomic alterations in genes frequently altered in IDH-wild-type glioma;

FIG. 4D is a heatmap of TCGA glioma samples ordered according to FIG. 2A and two EReg gene signatures defined for the IDH-wild-type DNA methylation clusters; mean RNA sequencing counts for each gene matched to the promoter of the identified cgID across each cluster are plotted to the right;

FIG. 4E is a heatmap of the validation set classified using the random forest method using the 1,300 probes defined in FIG. 2A;

FIG. 5 is a graphical and schematic representation of an integrative analysis of 1,122 adult gliomas resulted in 7 different subtypes with distinct biological and clinical characteristics; the groups extend across six DNA methylation subtypes of which the LGm6 cluster was further separated by tumor grade into pilocytic astrocytoma (PA)-like and LGm6-GBM; the size of the circles is proportional to the percentages of samples within each group; the DNA methylation schematic is a cartoon representation of overall genome-wide epigenetic pattern within glioma subtypes; survival information is represented as a set of Kaplan-Meier curves, counts of grade, histology and LGG/GBM subtypes within the groups are represented as bar-plots, whereas age is represented as density; labeling of telomere length and maintenance status is based on the enrichment of samples within each column, similarly for the biomarkers and the validation datasets;

FIG. 6A is a graph of RNaseq TERT expression is in TERTp mutant cases and in ATRX and double negative cases (p<0.0001);

FIG. 6B is a graph of TERT expression as quantified by RNA sequencing;

FIG. 6C is a graph of telomere length with age in tumor samples (p<0.0001);

FIG. 7A is a boxplot of the mean DNA methylation beta-values genome-wide (20,036 probes) for each sample distributed by the six Pan-glioma DNA methylation clusters (left) and divided by tumor type (right); Significant differences are highlighted with *(p-value <0.01) and ***(p-value <1e-04)

FIG. 7B is a graph of the principal component analysis of 932 TCGA glioma samples and 77 non-tumor brain samples (Guintivano et al., 2013) performed on 19,520 CpG probes (genome-wide);

FIG. 7C is a clustered heatmap of merged LGG-GBM mRNA data with 569 GBM and 463 LGG non-duplicate samples, and 2000 most variable genes;

FIG. 7D is a Functional Copy Number (fCN) gene signature heatmap; genes with Spearman's correlation between CN and Expression above 0.5, abs (FC>1.5) and abs(ΔCN>0.5) define the fCN signature; RNA expression levels range from green (low) to red (high).

FIG. 7E is a clustered heatmap of unsupervised hierarchical clustering of 473 samples (columns) and 196 antibodies (rows); the annotation bars (shown on top) were not used for clustering; the legend for the annotation bars is shown on the left; in the heatmap, low, medium, and high expression is represented by blue, white, and red colors, respectively;

FIG. 8A, left is a heatmap of DNA methylation data; unsupervised consensus clustering analysis using 1,308 CpG tumor specific CpG probes defined among the TCGA IDH mutant gliomas; column-wise represents 450 IDH mutant glioma samples, row-wise represents probes; samples are ordered according to the consensus cluster output, and rows are ordered by hierarchical clustering; DNA methylation beta-values ranges from 0 (low) to 1 (high); three clusters were defined, each cluster separated and labeled; non-tumor brain samples are represented on the left of the heatmap; additional tracks are included at the top of the heatmaps to identify each sample membership within separate cluster analysis (Glioma subtypes, tumor type, previous published subtypes, RNA sequencing and TERT expression); Right is a heatmap of clustering of IDH mutant samples transcriptional profiles; unsupervised clustering of gene expression separated by IDH status 426 samples confirming the presence of three main groups resembling the clusters previously reported in where all GBM G-CIMP cluster together with the LGG IDH mutant-non-codel;

FIG. 8B is a boxplot of the average DNA methylation beta-value genome-wide (20,000 probes) for each sample grouped by IDHmut K1 and IDHmut K2; dots represent LGG tumors and triangles represent GBM tumors; significant difference is highlighted with ***(p-value <2.2×10-16);

FIG. 8C, left is a heatmap of DNA methylation data; supervised statistical analysis using 149 CpG tumor specific CpG probes that define each TCGA IDH mutant glioma subtype; column-wise represents 448 IDH mutant (codels and non codels) TCGA glioma samples, row-wise represents probes; DNA methylation beta-values ranges from 0 (low) to 1 (high); Right is a heatmap of DNA methylation data for the validation dataset using the 149 CpG tumor specific probes that define each TCGA IDH mutant glioma subtype; non-TCGA glioma samples were classified into one of the three IDH mutant type specific clusters using the random forest machine learning method; DNA methylation beta-values ranges from 0 (low) to 1 (high); additional tracks are included at the top of the heatmap to identify tumor histology, published clusters (Published Clusters) and each sample membership according to its dataset (Study);

FIG. 8D is a set of Kaplan-Meier survival curves showing samples separated by IDHmut K1 low, IDHmut K1 high, IDHmut K2 and IDHmut K3; ticks represent censorship;

FIG. 8E is two graphs showing pathway analysis of differentially expressed genes between IDHmut K1, IDHmut K2, ranked by p-value; the top panel shows categories enriched in IDHmutK2; the bottom panel displays categories enriched in IDHmutK1;

FIG. 9A is a heatmap of DNA methylation data; unsupervised consensus clustering analysis using 914 CpG tumor specific probes defined among the TCGA IDH-wild-type gliomas; column-wise represents 430 IDH-wild-type TCGA glioma samples, row-wise represents probes; samples are ordered according to the consensus cluster output, and rows are ordered by hierarchical clustering; DNA methylation beta-values ranges from 0 (low) to 1 (high); three clusters were defined, each cluster separated and labeled; non-tumor brain samples are represented on the left of the heatmap; additional tracks are included at the top of the heatmaps to identify each sample membership within separate cluster analysis (Glioma subtypes, tumor type, previous published subtypes, RNA sequencing and TERT expression);

FIG. 9B is a heatmap of DNA methylation data for the validation dataset using the 914 CpG tumor specific probes defined in FIG. 9A; non-TCGA glioma samples were classified into one of the three IDH-wild-type specific clusters using the random forest machine learning method; the second track from top to bottom shows the classification of non-TCGA glioma samples into one of the seven glioma subtypes also using the random forest machine learning method; DNA methylation beta-values ranges from 0 (low) to 1 (high); additional tracks are included at the top of the heatmap to identify each sample membership according to its dataset (Dataset), to previous published clusters (Published Clusters) and to tumor histology;

FIG. 9C is a map of clustering of IDH-wild-type samples transcriptional profiles; unsupervised clustering of gene expression separated by IDH status showed that the LGr4 cluster identified in the pan-glioma unsupervised analysis splits into four mixed LGG/GBM clusters (234 samples), where the first two, although separated by a relatively small number of genes, are respectively enriched with Classical subtype (59%) and LGm4 samples and the second with Mesenchymal (75%) subtype and LGm5 samples, the third enriched with Proneural subtype (85%) and a fourth mostly containing LGG IDH-wild-type samples;

FIG. 9D is a boxplot of the estimate stromal score for each sample distributed by the four glioma IDH wild-type subtypes; significant differences are highlighted with *(p-value <0.05) and **(p-value <0.005);

FIG. 9E is an IGV screenshot showing differences in copy number landscape across glioma subtypes

FIG. 10A is a schematic diagram of the progression of LGG IDH mutant-non-codel to GBM G-CIMP in LGr3 as marked by a hyper-proliferation signature and four major gene sets groups related to cell cycle and hyperproliferation, DNA metabolic processes, response to stress and angiogenesis.

FIG. 10B is a schematic diagram similar to that of FIG. 10A in which the gene sets activated in the GBM are compared to the LGG component of LGr4 (IDH-wild-type) to identify an inflammation and immunologic response signature characterized by the activation of several chemokines and interleukins enriching sets involved in inflammatory and immuno response, negative regulation of apoptosis, cell cycle and proliferation, IKB/NFKB kinase cascade;

FIG. 10C is a diagram of differential regulatory networks describing differential molecular activities between GBM and LGG in LGr3; dichotomies were selected by only choosing those where samples form tight linearly separable clusters in the high dimensional genomic space; the size of the node is inversely proportional to the magnitude of the p-value computed by LIMMA for each differential; curated canonical MSigDB pathways significantly represented in these networks are listed below each network;

FIG. 10D is a diagram similar to that of FIG. 10C, but for LGr4;

FIG. 10E is a schematic overview diagram of the adopted pipeline for extracting significant pathways;

FIG. 10F is a diagram of the distribution of Estimate, Immuno and Stromal score by tumor type in the IDH-wild-type samples.

### DETAILED DESCRIPTION

The present disclosure relates to methods, kits, and programmed computers for classifying patients with adult diffuse glioma into one of seven glioma types based on IDH mutation status, DNA methylation cluster, RNA cluster, telomere length, telomere maintenance, and biomarkers. The present disclosure further includes methods of treating patients based on their classification into one of the seven glioma types.

The present disclosure is based, in part, on the development and verification of a new glioma classification scheme. Therapy development for adult diffuse glioma has previously been hindered by incomplete knowledge of somatic glioma driving alterations and suboptimal disease classification. To develop the present classification scheme, the complete set of genes associated with 1,122 diffuse grade II-III-IV gliomas was defined from The Cancer Genome Atlas and molecular profiles were used to improve disease classification, identify molecular correlations, and provide insights into the progression from low- to high-grade disease. Whole-genome sequencing data analysis determined that ATRX but not TERT promoter mutations are associated with increased telomere length. Recent advances in glioma classification based on IDH mutation and 1p/19q co-deletion status were recapitulated through analysis of DNA methylation profiles, which identified clinically relevant molecular subsets. A subtype of IDH mutant glioma was associated with DNA demethylation and poor outcome; a group of IDH-wild-type diffuse glioma showed molecular similarity to pilocytic astrocytoma (PA) and relatively favorable survival. Understanding of cohesive disease groups identified with particular glioma types may aid improved clinical outcomes.

The following are terms relevant to the present disclosure:

An “individual” or “subject” or “patient” herein is a vertebrate, such as a human or non-human animal, for example, a mammal. Mammals include, but are not limited to, humans, primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys.

In certain non-limiting embodiments, the patient suffers from a diffuse glioma.

A “pharmaceutical composition” as used herein is a small molecule, biological, or other composition of matter introduced into the patient's body. A pharmaceutical composition can be in a form suitable for introduction to the patient's body and can include other non-active components, such as excipients and stabilizers.

A “cancer therapy” as used herein may include any cancer treatment other than a pharmaceutical composition. A cancer therapy can include radiation therapies and surgeries.

An “effective amount” of a substance as that term is used herein is that amount sufficient to effect beneficial or desired results, including clinical results, and, as such, an “effective amount” depends upon the context in which it is being applied. In the context of administering a pharmaceutical composition to treat a glioma and/or administering a pharmaceutical composition to reduce at least one symptom of a glioma, an effective amount of a substance is an amount sufficient to prevent the progression of glioma or at least one symptom thereof or to treat or ameliorate a glioma or at least one symptom thereof by at 10%, 20%, 30%, 40%, 50%, 60%, 70%, 80%, 90%, 95%, 98% or 99%.

As used herein, and as well-understood in the art, “treatment” is an approach for obtaining beneficial or desired results, including clinical results. For purposes of this subject matter, beneficial or desired clinical results include, but are not limited to, alleviation or amelioration of one or more signs or symptoms, diminishment of extent of the glioma, stabilized (i.e., not worsening) state of the glioma, delay or slowing of glioma progression, and/or remission of the glioma. The decrease can be at least a 10%, 20%, 30%, 40%, 50%, 60%, 70%, 80%, 90%, 95%, 98% or 99% decrease in severity of complications, signs or symptoms or in likelihood of progression of the glioma.

As used herein “classification” is the assignment of a patient to a glioma type.

A “grade” as used herein refers to histopathologic classification that forms the basis of the World Health Organization (WHO) classification of CNS tumors. The present disclosure is intended to provide an alternative to this grade system.

Methods of Classifying Patients with Adult Diffuse Gliomas

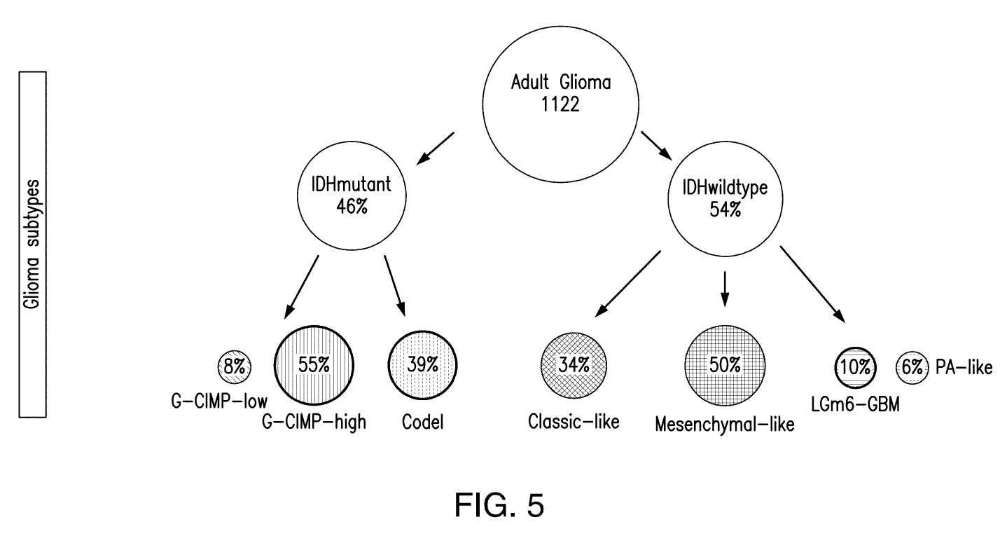

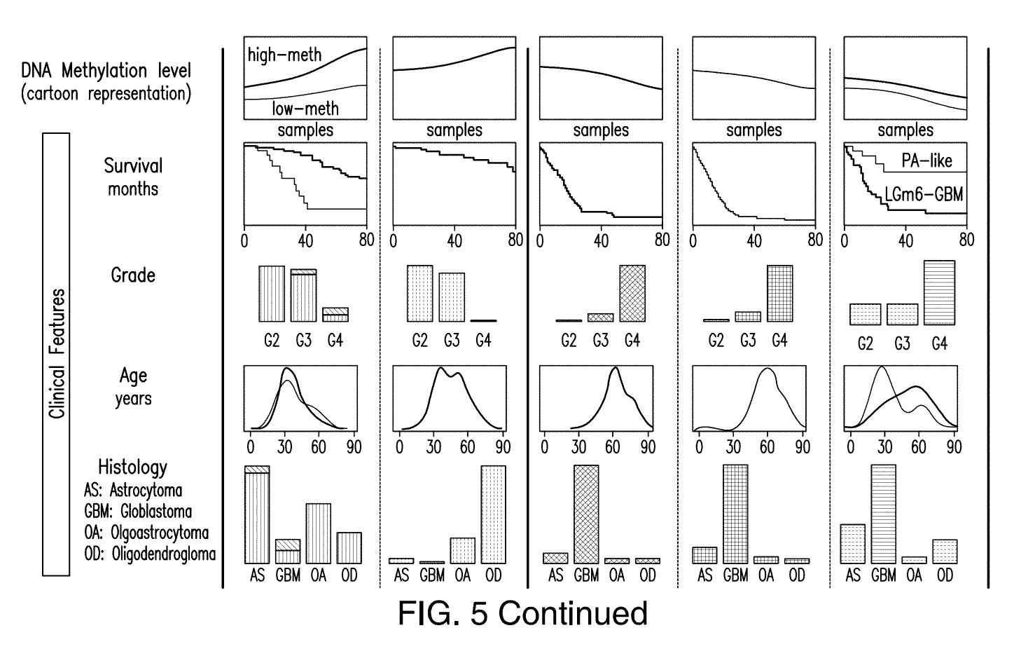

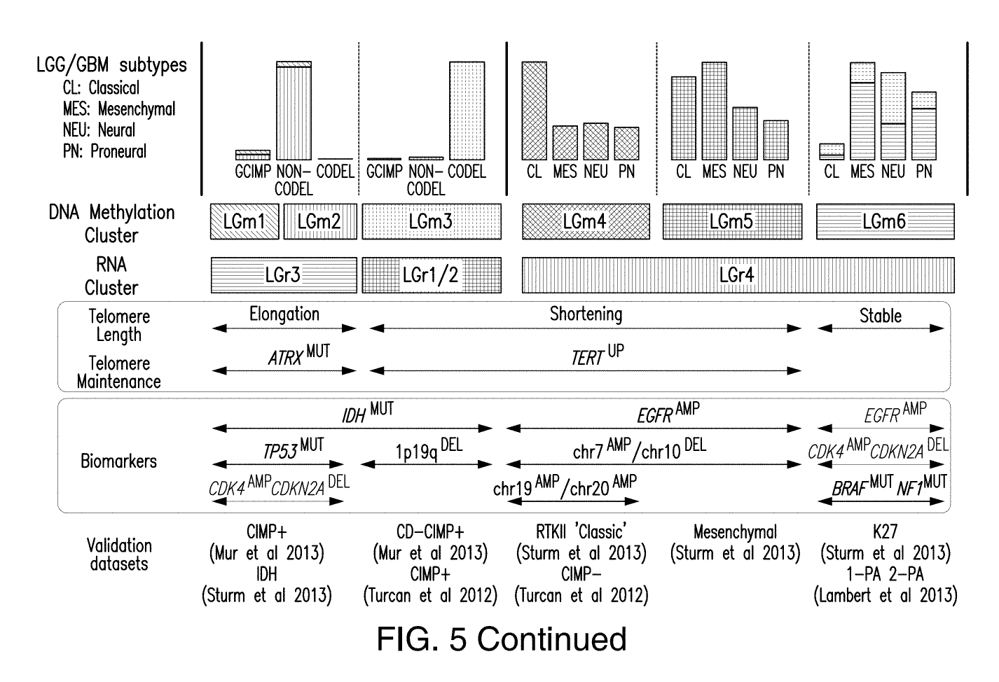

The present disclosure provides a method of classifying patients with adult diffuse glioma into one of seven glioma types. A summary of one non-limiting embodiment is provided in FIG. 5.

Using this method, gliomas are identified by IDH mutation status. An IDH mutant has a mutation in an IDH1 or IGH2 gene. According to certain non-limiting embodiments, an IDH mutant may have a mutation that results in hypermethylation of DNA. In certain non-limiting embodiments, the IDH mutant may have a point mutation. In certain non-limiting embodiments, the IDH mutant may have a codeletion of chromosome arm 1p and 19q (IDH mutant-codel) or be euploid 1p/19q (IDH mutant-non-codel).

Wild type IDH1 can have the sequence in Genbank Gene ID: 3417. Wild type IDH2 can have the sequence in Genbank Gene ID: 3418.

Gliomas are also identified by DNA methylation cluster as defined by TCGA. According to certain non-limiting embodiments, these clusters can include LGm1, LGm2, LGm3, LGm4, LGm5, and LGm6.

Gliomas are further identified by RNA cluster as further described in the Examples. According to certain non-limiting embodiments, these clusters can include LGr1/2, LGr3, and LGr4.

Gliomas are additionally identified by telomere length. According to certain non-limiting embodiments, telomere length may be described with respect to an appropriate comparative control, such as described in the Examples, as exhibiting elongation or shortening, or as stable.

Gliomas are additionally identified by telomere maintenance. According to certain non-limiting embodiments telomere maintenance may be classified based on an associated gene, particularly a mutation in ATRX or upregulation or TERT.

Gliomas are further identified by a set of biomarkers. According to certain non-limiting embodiments, the first biomarker is upregulation of Epidermal Growth Factor Receptor (EGFR). According to certain non-limiting embodiments, the second biomarker is a mutation in Tumor protein p53. According to certain non-limiting embodiments, the third biomarker is an IDH mutant-codel. According to certain non-limiting embodiments, the fourth biomarker is chromosome 7 (chr7) amplification coupled with a chromosome 10 (chr 10) deletion. According to certain non-limiting embodiments, the fifth biomarker is a Cyclin-dependent kinase 4 (CDK4) amplification coupled with a Cyclin Dependent Kinase Inhibitor 2A (CDKN2A) deletion. According to certain non-limiting embodiments, the sixth biomarker is a chromosome 19 (chr19) amplification and a chromosome 20 (chr20) amplification. According to certain non-limiting embodiments, the seventh biomarker is a B-Raf gene (BRAF) mutation coupled with a Neurofibromin 1 (NF1) mutation.

Identificaitons as described above may be performed as described in the Examples, or otherwise using DNA, RNA, protein, and chromosomal analysis platforms and methods.

A first glioma type is identified as IDH mutant/G-CIMP low. The IDH mutant/G-CIMP low glioma type exhibits an IDH mutation, the LGm1 DNA methylation cluster, the LGr3, RNA cluster, elongated telomere length, an ATRX mutation, a TP53 mutation, and a CDK4 amplification coupled with a CDKN2A deletion. The IDH mutant/G-CIMP low glioma type may fail to exhibit any of the other ARTX or TERT features or seven biomarkers noted above.

A second glioma type is identified as IDH mutant/G-CIMP high. The IDH mutant/G-CIMP high glioma type exhibits an IDH mutation, the LGm2 DNA methylation cluster, the LGr3 RNA cluster, elongated telomere length, an ATRX mutation, and a TP53 mutation. The IDH mutant/G-CIMP high glioma type may fail to exhibit any of the other ARTX or TERT features or seven biomarkers noted above.

A third glioma type is identified as IDH mutant/Codel. The IDH mutant/Codel glioma type exhibits an IDH mutation, the LGm3 DNA methylation cluster, the LGr1/2 RNA cluster, shortened telomere length, upregulation of TERT, and an IDH mutant-codel. The IDH mutant/Codel glioma type may fail to exhibit any of the other ARTX or TERT features or seven biomarkers noted above.

A fourth glioma type is identified as IDH wild type/Classic like. The IDH wild type/Classic like glioma type exhibits wild type IDH, the LGm4 DNA methylation cluster, the LGr4 RNA cluster, shortened telomere length, upregulation of TERT, amplified EGFR, amplified chr 7 coupled with a chr 10 deletion, and amplified chr 19 coupled with amplified chr 20. The IDH wild type/Classic like glioma type may fail to exhibit any of the other ARTX or TERT features or seven biomarkers noted above.

A fifth glioma type is identified as IDH wild type/Mesenchymal-like. The IDH wild type/Mesenchymal-like glioma type exhibits wild type IDH, the LGm5 DNA methylatin cluster, the LGr4 RNA cluster, shortened telomere length, amplified EGFR, and amplified chr 7 coupled with a chr 10 deletion. The IDH wild type/Mesenchymal-like glioma type may fail to exhibit any of the other ARTX or TERT features or seven biomarkers noted above.

A sixth glioma type is identified as IDH wild type/LGm6-GBM. The IDH wild type/LGm6-GBM glioma type exhibits wild type IDH, the LGm6 DNA methylation cluster, the LGr4 RNA cluster, stable telomer length, amplified EGFR, a CDK4 amplification coupled with a CDKN2A deletion, and a BRAF mutation coupled with a NF1 mutation. The IDH wild type/LGm6-GBM glioma type may fail to exhibit any of the other ARTX or TERT features or seven biomarkers noted above.

A seventh glioma type is identified as PA-like. The PA-like glioma type exhibits wild type IDH, the LGm6 DNA methylation cluster, the LGr4 RNA cluster, stable telomer length, and a BRAF mutation coupled with a NF1 mutation. PA-like glioma type may fail to exhibit any of the other ARTX or TERT features or seven biomarkers noted above.

The present disclosure further includes a kit for use in implementing any one or all of the above identifications. According to certain non-limiting embodiments, the kit can include reagents and other materials suitable for DNA and RNA extraction and analysis, including sequencing and, expression, mutation, or cluster analysis. According to certain non-limiting embodiments, the kit can include reagents and other materials suitable for protein extraction and analysis, including sequencing and identification of the quantity of protein. According to certain non-limiting embodiments, the kit can include reagents and other materials suitable for chromosome detection.

The present disclosure further includes a computer able to carry out the above methods to identify a glioma type using the above identifications. The computer can include a processor able to execute to identify a glioma type as well as a memory and an input module able to receive the identifications. According to certain non-limiting embodiments, the computer can receive the identifications as data from another computer via an input module. According to certain non-limiting embodiments, the computer can receive raw data and execute additional steps on the processor to create one or more identifications. The computer may further include an output to provide the glioma type and/or identifications to a user.

### Methods of Treatment Using Glioma Type

A patient classified with a glioma type as described above may be treated with cancer therapy or effective amount of a pharmaceutical composition based on the glioma type.

According to certain non-limiting embodiments, patients with the IDH mutant/G-CIMP low glioma type may receive increased treatment as compared with patients with the IDH mutant/G-CIMP high glioma type. According to certain non-limiting embodiments, patients with the IDH mutant/G-CIMP low glioma type may receive treatment appropriate for Grade 3 and Grade 4 gliomas.

According to certain non-limiting embodiments, patients with the IDH mutant/G-CIMP high glioma type may receive treatment appropriate for Grade 2 or Grade 3 gliomas, with treatment appropriate for Grade 4 gliomas only if other factors so indicate.

According to certain non-limiting embodiments, patients with the IDH mutant/Codel glioma type may receive treatment appropriate for Grade 2 or Grade 3 gliomas, with treatment appropriate for Grade 4 gliomas only if other factors so indicate.

According to certain non-limiting embodiments, patients with IDH wild type/Classic like glioma type may receive treatment appropriate for Grade 4 gliomas, with treatment appropriate for Grade 2 or Grade 3 gliomas only if other factors so indicate.

According to certain non-limiting embodiments, patients with IDH wild type/Mesenchymal-like may receive treatment appropriate for Grade 4 gliomas, with treatment appropriate for Grade 2 or Grade 3 gliomas only if other factors so indicate.

According to certain non-limiting embodiments, patients with IDH wild type/LGm6-GBM may receive treatment appropriate for Grade 4 gliomas.

According to certain non-limiting embodiments, patients with PA-like glioma type may receive treatment appropriate for Grade 2 or Grade 3 gliomas.

According to certain non-limiting embodiments, the dissection of the IDH mutant non-codel G-CIMP LGG and GBM into two separate subgroups (G-CIMP-low and G-CIMP-high) based on the extent of genome-wide DNA methylation has clinical relevance. In particular, the identification of the G-CIMP-low subset, characterized by activation of cell cycle genes mediated by SOX binding at hypomethylated functional genomic elements and unfavorable clinical outcome, allows more accurate segregation and therapeutic assessment in a group of patients in which correlations of conventional grading with outcome are modest (Olar et al., 2015, Reuss et al., 2015).

According to certain non-limiting embodiments, the finding that G-CIMP-high tumors can emerge as G-CIMP-low glioma at recurrence identifies variations in DNA methylation as an important determinants for glioma progression.

According to certain non-limiting embodiments, the identification of a PA-like LGG subset that harbors a silent genomic landscape, confers favorable prognosis relative to other IDH-wild-type diffuse glioma, and displays a molecular profile with high similarity to PA, allows less rigorous treatment of such patients.

### EXAMPLES

The presently disclosed subject matter will be better understood by reference to the following Examples, which are provided as exemplary of the disclosure, and not by way of limitation.

### Materials and Methods Used in Examples

The following materials and methods were used in these examples.

Platforms

In total, tumors from 1,132 patients were assayed on at least one molecular profiling platform, which platforms included: (1) whole-genome sequencing, including high coverage and low pass whole-genome sequencing; (2) exome sequencing; (3) RNA sequencing; (4) DNA copy-number and single-nucleotide polymorphism arrays, including Agilent CGH 244K, Affymetrix SNP6.0, and Illumina 550K Infinium HumanHap550 SNP Chip microarrays; (5) gene expression arrays, including Agilent 244K Custom Gene Expression, Affymetrix HT-HGU133A and Affymetrix Human Exon 1.0 ST arrays; (6) DNA methylation arrays, including Illumina GoldenGate Methylation, Illumina Infinium HumanMethylation27, and Illumina Infinium HumanMethylation450 BeadChips; (7) reverse phase protein arrays; (8) miRNA sequencing; and (9) miRNA Agilent 8×15K Human miRNA-specific microarrays. Details of data generation have been previously reported (Brennan et al., 2013, Cancer Genome Atlas Research Network et al., 2015). To ensure cross-platform comparability, features from all array platforms were compared to a reference genome.

Biospecimens

Biospecimens were collected from human patients diagnosed with low grade gliomas (LGG) and glioblastoma multiforme (GBM) undergoing surgical resection. The case list freeze included 1122 cases comprising 516 LGG and 606 GBM. Samples were acquired and processed according to previous descriptions (Brennan et al., 2013; TCGA_Network, 2015). Clinical data elements available for the patients included histology, grade, gender, age at diagnosis/surgery, treatments, vital status, overall and progression-free survival. Overall survival was defined as the time from surgical diagnosis until death. Patients who were still alive at the time of this study had overall survival time censored at the time of last follow-up.

Survival curves were estimated and plotted using the Kaplan-Meier method. Log-rank tests were used to compare curves between groups. Single-predictor and multiple-predictor models were fit using Cox regression under the proportional hazards assumption. Hazard ratios and 95% confidence intervals are reported. Nested models were compared using the likelihood ratio test (LRT). Harrell's concordance index (C-index) was used to assess and report model performance (Harrell et al., 1982). These analyses were conducted in R (v 3.1.2) using the survival package (Therneau, 2014; Therneau and Grambsch, 2000).

DNA Sequencing Data Production

Whole exome, whole genome and targeted validation and TERT promoter sequencing (including low-pass sequencing) was performed as previously described (Brennan et al., 2013; Cancer Genome Atlas Research, 2015; Verhaak et al., 2010).

Identification of Somatic Mutations

The Broad Institute's Firehose cancer genome analysis pipeline used BAM files for tumor and matched normal samples to perform quality control, local realignment coverage calculations and others on whole exome sequencing (Table 1) as described (Imielinski et al., 2012). For the identification of somatic single nucleotide variations a multicenter approach integrating the output of three different somatic mutation algorithms: MuTect (Cibulskis et al., 2013), RADIA (Radenbaugh et al., 2014) and Varscan (Koboldt et al., 2012) was used. MAF files from each mutation calling algorithm were integrated in a unique MAF file considering those mutations that were called at least by two of the three considered methods. The integrated MAF contains 28637 somatic mutation called by all the methods, 5559 called by MuTect and VarScan, 7971 called by MuTect and RADIA and 730 called by VarScan and RADIA. Similarly, for the detection of somatic insertions and deletions the calls produced by Indelocator and Varscan algorithms were intersected, obtaining 1956 high confidence indels.

Identification of IDH Mutations

In order to expand the annotation of IDH status in the test cohort, previously reported (Cancer Genome Atlas Research, 2008) mutation calls on Sanger sequenced DNA and exome sequencing of whole genome amplified DNA were used. Sanger sequencing and whole exome sequencing of whole genome amplified DNA was performed as previously described (Brennan et al., 2013; Cancer Genome Atlas Research, 2008; Verhaak et al., 2010). Except for bona fide IDH1/2 mutations, no other mutations were called on these platforms. An ABI platform with a DNA sample for GBM was performed with 158 paired samples. An Illumina platform with WGA for GBM was performed with 163 paired samples. There was a union of 174 paired samples. Additional data used to determine IDH mutation status.

Identification of TERT Promoter Mutations

Targeted sequencing at the TERT promoter region (Chr5:1295150-1295300) was performed on a subset of 287 cases as previously described (Cancer Genome Atlas Research, 2015). Additionally, whole genome sequencing (including low-pass) was evaluated for the presence of somatic variants using GATK pileup. A minimum coverage of at least 6 bp and a minimum variant allele faction of 15% was required for detection of TERT promoter mutations. A total of 328 cases had sufficient coverage to detect a mutation and 162 cases showed a somatic mutation at one of three sites.

A nucleotide change of A161C at Chr5:1295161 was seen in 2 paired samples. A nucleotide change of C228T at Chr5:1295228 was seen in 121 paired samples. A nucleotide change of C250T at Chr5:1295250 was seen in 39 paired samples. One patient showed mutations in both C250T and C228T.

Mutation Significant Analysis

Significantly mutated genes were identified using the MutSigCV algorithm. Analyses were conducted on the entire sample set (n=820) accept a single hypermutator phenotype (TCGA-DU-6392). Intronic mutations were excluded. A mutation blacklist was applied for remove potential technical artifacts (Lawrence et al., 2013b). Genes with a q-value less than 0.1 were considered significant.

Telomere Quantification

Quantification of telomere length was performed using the TelSeq tool (Ding et al., 2014). This tool counts the number of reads containing any (range 0 to k) amount of telomeric repeats (nk), or TTAGGG, and then computes the estimated telomere length in bp 1 further based on the average chromosome length in bp c and the total coverage s.

l=c×nk/s 1)

Ding et al. recommend a k of 7 based on their experimentally validated results. Given that TelSeq was not designed for cancer, it does not take into account tumor ploidy and purity. The TelSeq computation was therefore modified to consider tumor purity p and ploidy τ:

nk/s=lt×τ×p+ln×(1−p)/τ×c×p+c×(1−p) 2)

Because p and τ are given by the ABSOLUTE analysis (Carter et al., 2012), solving lt is straightforward, whereas ln can be calculated using 1) above.

The average chromosome length c is calculated as follows:

c=46/G 3)

Here G is the total genome length and 46 is the expected number of chromosomes. Because GC content is a potential confounding factor, G was set to the genome length in bp with GC content between 48% and 52%. The average coverage s is adjusted in a similar fashion.

Whole Genome Mutation Calling

MuTect (Cibulskis et al., 2013) was used to call somatic mutations on 89 matched primary tumor normal pairs. a minimum coverage of 14 in the tumor sample and 8 in the normal sample was required. Variants known to dbSNP v132 and unknown to COSMIC v54 were filtered resulting in 714,305 variants. Using these samples overlapping RNA-seq expression data was used to form an integrated dataset of 67 pairs (29 GBM, 38 LGG). In order to identify potential promoter sites the GENCODE v19 transcript annotation (n=196,520 transcripts) was used with a subset of 24,001 transcripts that have an exact UniProt database match and has been curated according to known clinically relevant protein changes (Ramos et al., 2015). The transcripts were then reduced down to one transcript per gene (n=17,722 transcripts). For each remaining transcript a region spanning from 2,000 bp upstream of the transcription start site and 200 bp into the coding region was examined. Overlapping mutations were determined for each region using the Bioconductor package “GenomicRanges” (Lawrence et al., 2013a). Regions with hits from less than 7 unique samples, regions which were upstream of genes lacking RNA-seq counts or counts that were lacking any variability, regions in which the variants had a median of read count of 1 or more alternate reads in the matching normal were removed. This filtering resulted in 141 mutations across 12 putative promoter regions. For each of the remaining gene promoter regions a t-test and a mann-whitney-U test comparing the log 2 normalized gene expression counts in mutant cases to wild type cases were performed. Promoter regions were subsequently filtered out with a Benjamini-Hochberg adjusted gene expression correlation Q-value <0.05, and only three promoter regions remained including TERT, TRIM28 and CACNG6.

Preprocessing and Peak Calling

Tumor and normal samples were profiled on Affymetrix SNP6.0 GeneChip arrays and subsequently processed into genome segmentation files (McCarroll et al., 2008). The tool GISTIC 2.0 was then used to identify significantly reoccurring focal and broad copy number changes (Mermel et al., 2011). Events with a Q-value <0.10 were considered significant. In order to identify low-frequent subtype specific events, GISTIC was run both across the entire cohort (n=1084) and smaller subsets within DNA methylation clusters (n=6 groups), RNA expression clusters (n=4 groups) and IDH-codel subtypes (n=3 groups). For each statistically significant peak, GISTIC 2.0 indicates a narrow focal peak and a wider surrounding peak. All overlapping focal peaks across all GISTIC run and were intersected and 57 disjoint amplified regions and 105 deleted regions were identified. Using this method, while drastically limiting the number of genes compared to using the wide peak boundaries, 80% of genes that were considered as potential tumor drivers in previous studies were still located.

Genes previously suggested as tumor drivers not found using this method include IRS2 gain, LSAMP loss and KDR/KIT gain (the neighboring oncogene PDGFRA however was still found). In order to further narrow down the list of genes per peak and to identify potential tumor drivers, copy number change was correlated to gene expression and prioritized genes in which significant mutations were found. Using this method, evidence for several new tumor drivers including GIGYF2 loss, ERRFI1 loss, ARID2 loss and FGFR2 gain, was found.

Functional Copy Number (fCN) Analysis

In order to define the functional copy number (fCN) genes the Spearman's correlation between the copy number and the expression of each gene in the dataset was calculated. All the genes with p-value <0.05 and cor >0.5 were selected.

In order to highlight the different behavior between the four expression groups, only the differentially expressed (abs (FC>1.5)) and aberrated (abs (ΔCN>0.5)) fCN genes were selected, resulting in a list of 57 genes (the fCN signature).

Mutations with Common Focal Alterations (MutComFocal)

By considering both copy number and somatic mutation data of LGG/GBM samples, the algorithm of MutComFocal (Trifonov et al., 2013) was applied. Particularly, focality score and recurrence score were calculated based on samples with at least 10 and at most 1,000 copy number segments. The focality score assigns equal weight to all genes participating in a genomic alteration inversely proportional to the size of that alteration, while recurrence score assigns equal weight to all genes altered in a sample inversely proportional to the total number of gene altered in the sample (Frattini et al., 2013; Trifonov et al., 2013).

### RNA Data Preparation and Gene Selection

RNA-seq raw counts of 667 cases (513 LLG and 154 GBM) were downloaded, normalized and filtered using the Bioconductor package TCGAbiolinks (Colaprico et al., 2015) using TCGAquey( ), TCGAdownload( ) and TCGAprepare( ) for both tumor types (“LGG” and “GBM”, level 3, and platform “IlluminaHiSeq_RNASeqV2”). The union of the two matrices was then normalized using within-lane normalization to adjust for GC-content effect on read counts and upper-quantile between-lane normalization for distributional differences between lanes applying the TCGAanalyze_Normalization( ) function encompassing EDASeq protocol. Gene selected for clustering were chosen by applying two filters, the first was aimed a reducing the batch effect between the two tumor cohorts. Differentially expressed genes were computed with TCGAanalyze_DEA( ) (implementing the EdgeR protocol (Robinson et al., 2010)), and genes differentially expressed between the two sets (α=10-10) were filtered out, obtaining 10,389 genes. Variability filters that select genes having a sufficiently high variation (100%) between the mean of top 5% and the mean of the bottom 5% values and having these means respectively above and below the overall median value of the data matrix were applied. The filtering steps resulted in 2,275 genes that were used for the consensus clustering. ConsensusClusterPlus Bioconductor package was used to perform the clustering with hierarchical clustering as inner method and 1000 resampling steps (epsilon=0.8). Number of cluster (n=4) was used as local maxima of the Calinsky-Harabasz curve. Within cluster analysis was done generating differentially expressed genes between GBM and LGG cohorts (log fold change greater and 1.0 and FDR less than 0.05), lists were then analyzes using DAVID functional annotation tool (Huang et al., 2009) and ClueGO (Bindea et al., 2009).

Classification of Affymetrix Samples

Once the four RNA-seq cluster were obtained, 378 GBM samples for which no RNAseq data were available were reclassified using their Affymetrix profiles. The 151 GBM samples (20 in LGr1, 4 in LGr2, 10 in LGr3 and 117 in LGr4) having both the Affymetrix and RNA-seq profiles were used as a training set of a kNN classifier (k=3) to assign LGr cluster memberships to the remaining 378 Affymetrix samples. The feature set of the classifier was based on a signature of 327 probesets obtained by selecting up-regulated and down-regulated genes for the training samples in each cluster.

Combining Multi-Platform Multi-Tumor Datasets

The ComBat batch effect removal method (Johnson et al., 2007) was used in order to combine mRNA expression data from the GBM RNA-seq (n=154), GBM Agilent (n=525), LGG RNA-seq (n=513), and LGG Agilent (n=27) datasets. Data generated using Agilent microarray platform was used preferentially over those generated using Affymetrix because such data were available for both tumor types, while Affymetrix data were only available for GBM samples. The 4 datasets were combined and analyzed using ComBat. 4 batches were flagged, one for each dataset, to be removed by the ComBat method. One hundred and forty nine GBM samples were analyzed using both Agilent and RNA-seq platforms. Twenty seven LGG samples were analyzed using both Agilent and RNA-seq platforms. These matched samples were used as biological covariates in the ComBat method. Upon completion of the data transformation, all redundant samples analyzed using the Agilent platform were removed whenever the sample was also analyzed using RNA-seq. This combined mRNA expression dataset (n=1043) was used for Tumor Map analysis.

Tumor Map Method

Tumor Map is a dimensionality reduction and visualization method for high dimensional genomic data. It allows viewing and browsing relationships between high dimensional heterogeneous genomic samples in a two-dimensional map, in a manner much like exploring geo maps in Google Maps web application.

Prior to the analysis, technical and batch effects in the gene expression data were mitigated as a preprocessing step and as described above. Sample-by-sample pair-wise similarities were calculated. 6002 genes whose expression was the most variable based on the variance distribution curve were selected from RNA expression data. The 1301 most important methylation probes were selected by manual curation of the probe list as described in the DNA methylation analysis section.

Spearman rank correlation (Spearman, 1904) was used on these continuous variable data (mRNA and methylation). To build maps based on a single data type, for each sample the closest neighborhood of 10 samples was selected. The Tumor Map method represents these local neighborhoods as a graph. The edge weight in this graph is proportional to the magnitude of the similarity metric. Then spring-embedded graph layout (Golbeck and Mutton, 2005) algorithm is applied to the constructed graph. The spring-embedded layout algorithm treats edges as springs and allows the springs to oscillate for a fixed amount of time with the energy inversely proportional to the edge weights. Under these conditions, springs with large weights do not oscillate much, causing those vertices to stay together. However, springs with small weights oscillate more and end up farther away from each other. The method then projects the positions of all the vertices in the resulting graph layout onto a two-dimensional grid. Each cell in the grid allows only one vertex to be placed into it. If multiple vertices contest for the same grid cell, a random vertex selection is made and placed into the cell; and the other competing vertices are placed into the nearest empty cell, snapping around the original cell in a spiral-like manner. Thus, dense clumps of samples are separated so that they can be viewed at approximately the same scale as the distances that separate them. After computing pairwise sample similarities in the gene expression and DNA methylation space separately, the two similarity spaces were combined after standardizing each space.

Multi-platform maps using Bivariate Standardization similarity space Transformation (BST)

Sample pairwise similarities were computed for each data type separately, producing a square samples-by-samples similarity matrix. For each of the similarity matrices, bivariate standardization was performed by transforming each value to be an arithmetic mean of the z-scores of this value within both the row and the column empirical distributions. This method is an adaptation of the approach by Faith et. al (2007). Once each of the similarity matrices is transformed into a z-score space, each available z-scores (from N platforms) for each pair of samples were combined by taking a weighted average of the z-scores, where the weights indicate the importance of each of the N platforms being combined. When genomic data for a given platform was not available for at least one of the samples from the pair, a pairwise similarity for this pair was not available for this platform. This method allows such omissions, as it will only combine similarity z-scores from those platforms that are available for any pair of samples. The resulting BST matrix is a square samples-by-samples matrix that contains a union of samples in all the platforms.

Extracting Significantly Active Pathways

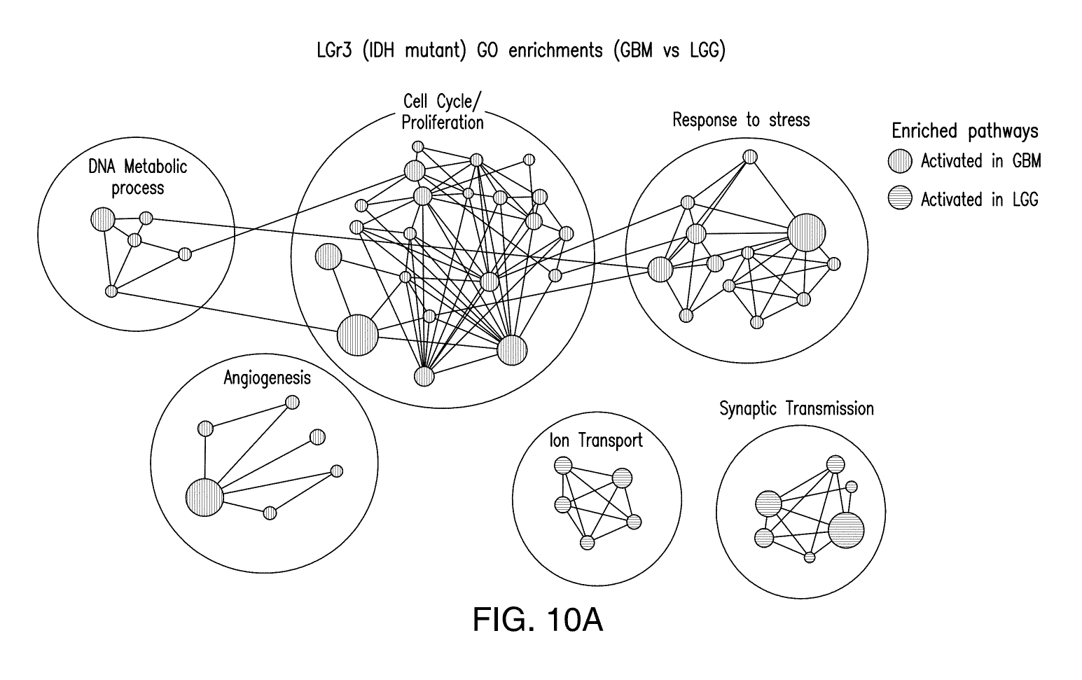

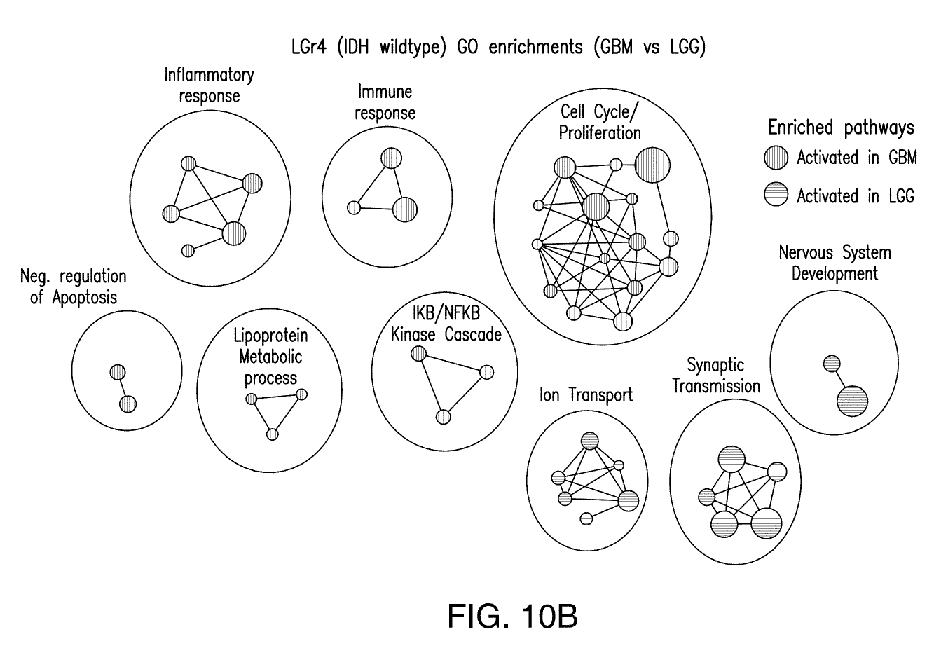

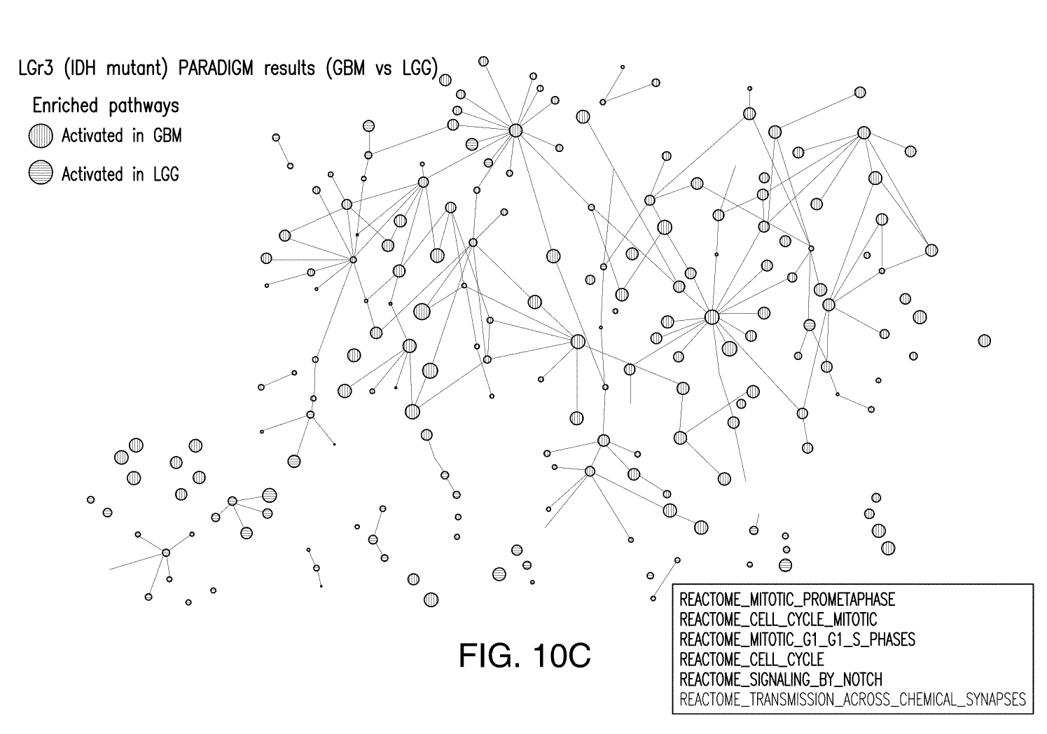

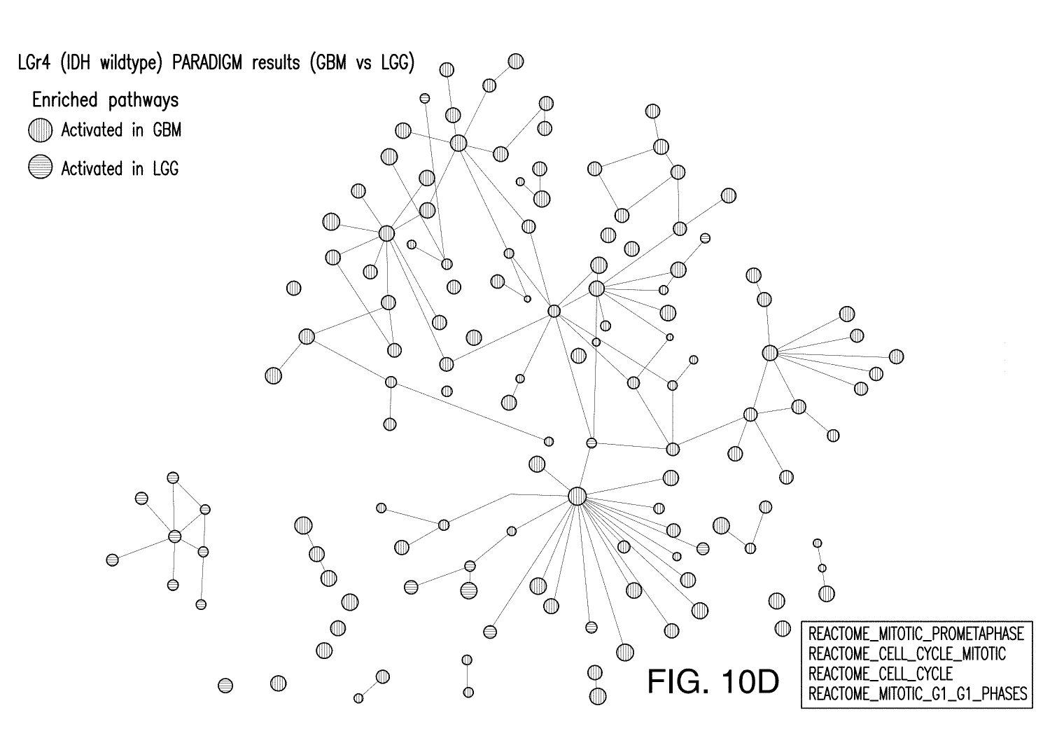

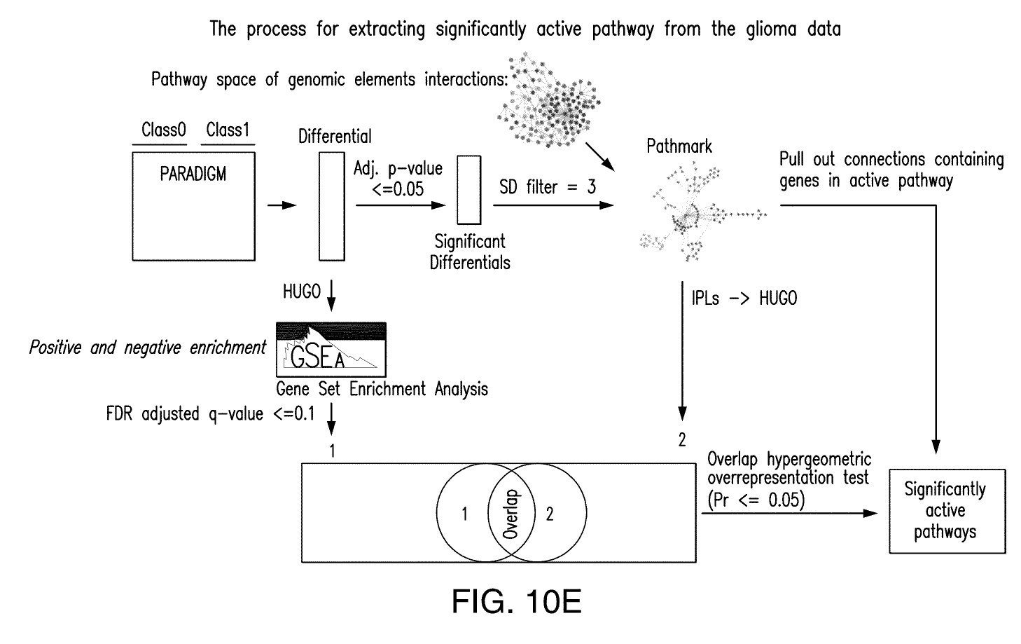

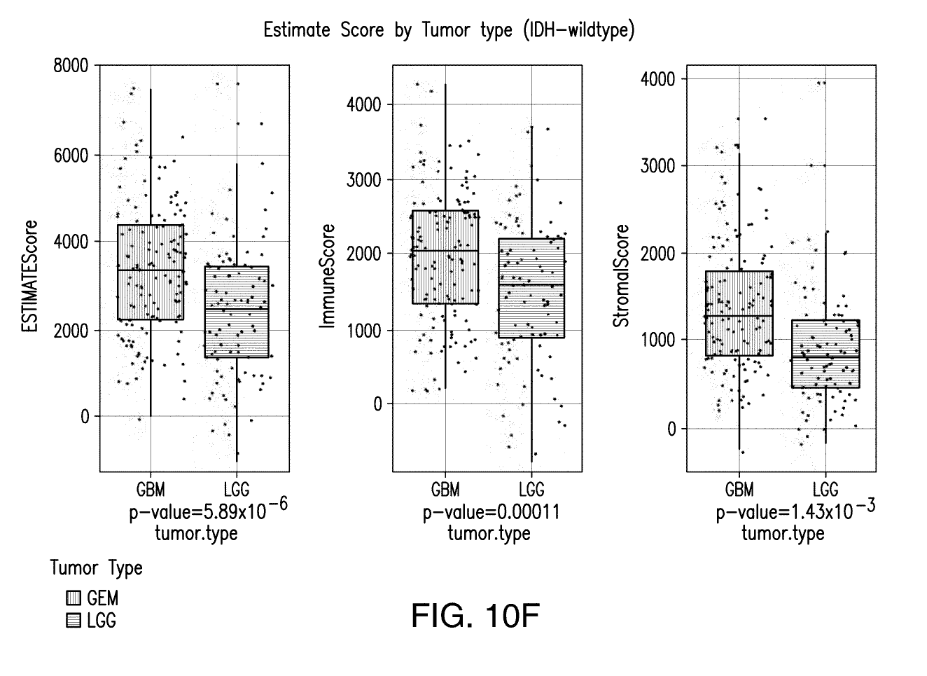

mRNA expression for samples available through RNA-seq platform only and the CNV data were used to transform the data into inferred pathway activity levels using PARADIGM (Vaske et al., 2010). A number of dichotomies, such as LGm1 GBM vs. LGG were then considered. Some of the dichotomies considered had significantly different numbers of samples in each class. In order to make statistically strong inferences about pathway activities only those dichotomies in which both classes were well represented by their members and the variance within the classes was much smaller than the variance between the classes were considered. Those dichotomies where sample scatter is small within the classes and classes are separable in the pathway space were considered. Based on the PARADIGM IPLs (Inferred Pathway Levels) pair-wise Spearman rank correlation for each pair of samples was computed. Within-class and between-class variance of the correlations were computed, first for the first class and then for the second class. The F-statistic for each of the classes in the dichotomy and the p-value based on the F-distribution were then also computed. The p-value for the dichotomy was determined by computing the mean p-value. Those dichotomies that had an aggregated p-value of <=0.05 were selected. For each dichotomy selected, differential activity levels were calculated using the linear models for microarrays and RNA-seq data (LIMMA) method (Smyth, 2005). Gene Set Enrichment Analysis (GSEA) (Subramanian et al., 2005) was then applied to the HUGO members of the full differential vector. Only those pathways that had FDR-adjusted q-value of <=0.1 were extracted. At the same time, statistically significant differentials (multiple hypothesis adjusted p-value <=0.05) were extracted. PATHMARK (Cancer Genome Atlas Research, 2013) was run on the statistically significant differential activities obtained from LIMMA to extract connected components of the global PARADIGM regulatory network. An additional filter of 3 standard deviations was applied to the PATHMARK method. This means only those activities that fall outside 3 standard deviations of the empirical distribution of the statistically significant differentials pass through the filter. A network connection is extracted if both vertices connected by that connection pass the filter. For each pathway gene set that passed the GSEA q-value of 0.1 the overlap of the pathway genes and those that survived the PATHMARK filter as well as the over-representation hypergeometric p-value was computed. Those pathways that passed with the p-value of <=0.05 were extracted. FIG. 10E shows an overview of the process for extracting significantly active pathway from the glioma data. FIG. 10C and FIG. 10D show pathway views of the significant IPLs in which IPLs representing families, complexes, phopho-events and redundant complexes were removed for better visualization.

Combining GBM Agilent G4502A mRNA Data with LGG Illumina Hi-Seq RNA-Seq Data

Approximately 15,700 genes were common between the two platforms and a total of 185 pairs of GBM and LGG sample replicates were run on both platforms. Initial tests by combining the GBM and LGG replicates and clustering them showed two clusters based entirely on platform differences and the replicates didn't merge with each other. To remove the platform effect, a novel algorithm that randomly divided the 185 replicate pairs into training, testing and validation sets was used. The training set was used to train an Empirical Bayes (Johnson et al., 2007) based model, which was then applied to the testing set. The testing set was used to figure out which genes didn't merge well by using a t-test to find the genes with the most differences between the platforms. The process was repeated 1000 times by using a bootstrapping approach for the training set. The top 3000 genes that were consistently found to be the most variable in the testing set were removed from the data set. The resulting model was then applied to the validation set, after removing those 3000 genes, to evaluate the algorithm. The evaluations showed that all 43 of the replicate pairs in the validation set clustered in matched pairs. The median of Pearson's correlations between the matched pairs was 0.23 before adjustment and 0.93 after adjustment, indicating very successful merging. The model was then applied to the full GBM and LGG dataset to perform overall merging, and then duplicates were removed by randomly keeping one sample from the pairs. The final dataset had 1032 samples and 12,717 genes.

Fusion Transcript Detection Using PRADA

Transcript fusions were detected in 665 samples using the Pipeline for RNA-seq Data Analysis (PRADA) fusion detection tool (Tones-Garcia et al., 2014). Fusions were classified to one of four tiers based on the number of junction spanning reads and discordant read pairs, gene partner uniqueness, gene homology, whether the fusion preserves the open reading frame, transcript allele fraction and DNA breakpoints in SNP6 array data, as previously described (Yoshihara et al., 2014).

Briefly, tier one fusions are the highest confidence fusions and tier four fusions are the lowest confidence ones. For the purpose of this analysis include tiers one and two were included.

Fusion Transcript Detection Using deFuse

RNA-seq reads were analyzed using deFuse package version 0.6.0 (McPherson et al., 2011). Fusions involving receptor tyrosine kinase genes were manually reviewed using blat analysis (Kent, 2002) of the breakpoint sequence in the UCSC Genome Browser (Kent et al., 2002). Candidate fusions were filtered based on the following deFuse parameters:
Splitr_count >=5 (5 or more split reads supporting the fusion)
Span_count >=10 (10 or more spanning reads supporting the fusion)
Read through˜“N” (fusion is not a readthrough)
Adjacent˜“N” (fusion does not involve adjacent genes)
Altsplice˜“N” (fusion cannot be explained by alternative splicing)
Min_map_count=1 (at least one spanning read supporting the fusion is uniquely mapped)
ORF˜“Y” (fusion preserves the open reading frame)

deFuse and PRADA fusion predictions were combined to generate a list of 204 events identified by both methods.

Identification of Transcriptional Regulatory Factors Underlying IDH Wild Type and IDH Mutant Phenotypes in Glioma

Batch-corrected expression data from Agilent Microarray and Illumina Hiseq RNA-seq platforms using MBatch was used for differential expression and transcription factor analysis. Linear regression was used to find the genes that are differentially expressed between IDH wild type and IDH-mutant groups after adjusting for the effect of expression platform (RNA-seq or microarray) in the model. The p-values are adjusted for multiple testing using the Bonferroni method. Genes with adjusted p-value less than 0.01 are considered significant.

Transcription Factor (TFs) Analysis was performed using the Match Algorithm of Biobase (TRANSFAC) system to identify TFs enriched in promoters of genes differentially expressed between IDH wild type and mutant groups. This algorithm compares the number of TF binding sites found in a query sequence set against a background set and identifies factors whose frequencies were enriched in the query compared to the background. Genes significantly upregulated in the IDH mutant group were considered as the background for TF analysis of genes upregulated in IDH wild type group and vice-versa. The TFs enriched with p-value less than 0.05 are considered significant.

Differential expression analysis was used to assess the expression differences of the enriched TFs themselves between the two groups (IDHmut vs wt). The transcription factors with Bonferroni adjusted p-value less than 0.05 are defined as significant candidates.

Ingenuity Pathway Analysis (IPA) was used to generate downstream networks for the top ranking transcription factors. Rank of the transcription factor is defined based on fold change between the two groups and the number of transcription factor binding sites in the promoter region of the target genes. Twelve transcription factor families were found to have log fold change of >1 between the IDH mut and IDHwt groups. The ones with the highest number of target genes are NKX2-5, PAX8, ETV7, CEBPD, ETV4, ELF4, and NFE2L3. Several of these TFs have been shown to be important for carcinogenesis. For example, Pax8 has been shown to be minimally expressed in LGG and normal brain but highly expressed in glioblastoma (Hung et al., 2014) and plays a role in telomerase regulation (Chen et al., 2008). Similarly, enrichment of the pro-proliferative TF ETV4 in 1p/19q codeleted gliomas has been demonstrated (Gleize et al., 2015).

Preprocessing and Clustering

For data acquisition, the Bioconductor package TCGAbiolinks (Colaprico et al., 2015) was used. First, TGCAquery( ) was used to search the samples of “GBM” and “LGG” tumors in TCGA repository using the following parameters: data level=3, platform type=“HumanMethylation450” and “HumanMethylation27”, version 12 for LGG and version 5 GBM samples. Second, TCGAdownload( ) was used to download the data; and, finally, TCGAprepare( ) was used to read the data into a dataframe. A total of 932 TCGA glioma samples assessed for DNA methylation, including 516 LGG and 416 GBM samples, profiled using 2 different Illumina platforms, were included. During the initial phase of the TCGA project, 287 GBM samples (batches 1 to 9) were profiled using the Illumina HumanMethylation 27 platform (HM27), which interrogates 27,578 CpG probes. As a new platform became available, the TCGA LGG (batches 1 to 16) and 129 GBM (batches 1 to 12) samples were transitioning into the larger more comprehensive Illumina platform known as the HumanMethylation450 (HM450), which interrogates 485,421 CpG sites. The DNA methylation score for each locus was presented as a beta (β) value (β=(M/(M+U)) in which M and U indicate the mean methylated and unmethylated signal intensities for each locus, respectively. β-values ranged from zero to one, with scores of zero indicating no DNA methylation and scores of one indicating complete DNA methylation. A detection p-value also accompanied each data point and compared the signal intensity difference between the analytical probes and a set of negative control probes on the array. Any data point with a corresponding p-value greater than 0.01 is deemed not to be statistically significantly different from background and is thus masked as “NA” in TCGA level 3 data packages. Data of the two platforms (HM450 and HM27) were merged as previously described (Brennan et al., 2013) and to arrive at 25,978 probes that match both 27 k and 450 k platforms. Duplicated samples and secondary tumors were excluded.

Unsupervised Clustering Analysis of DNA Methylation Data

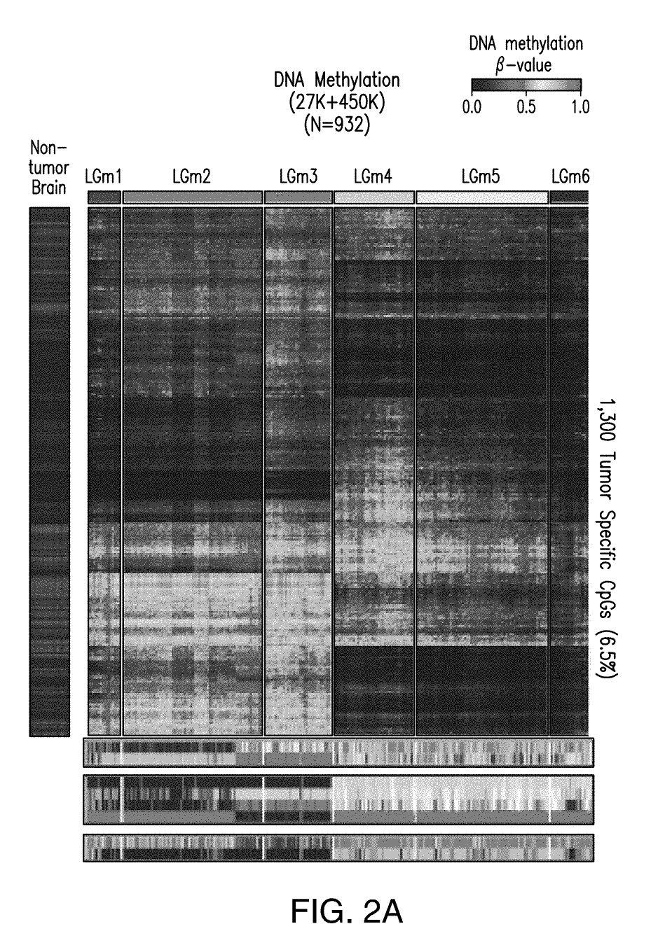

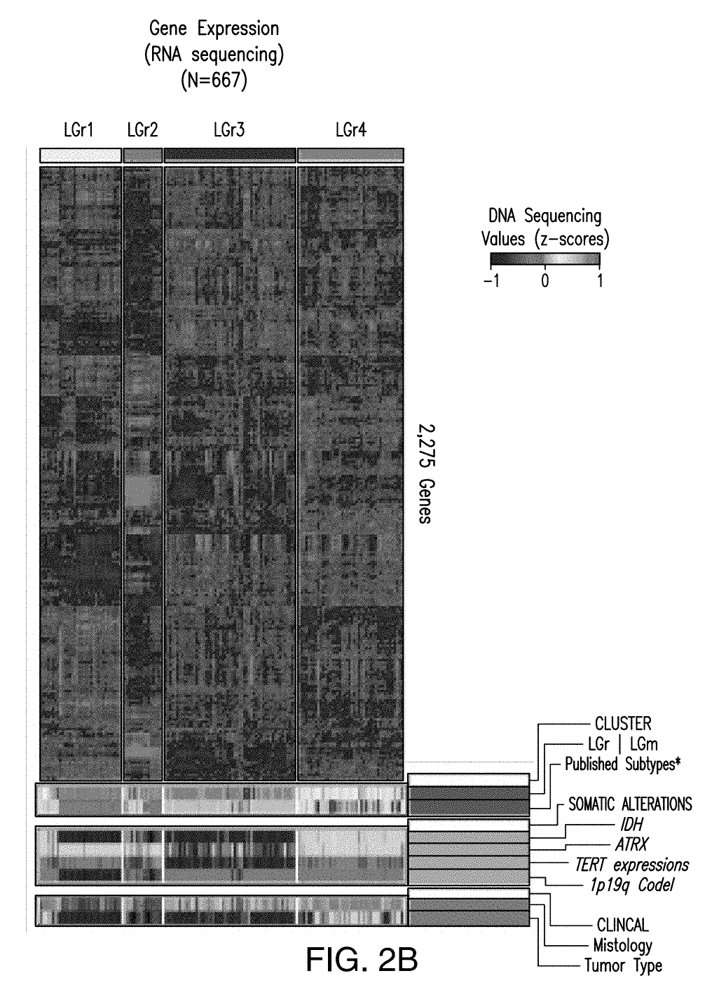

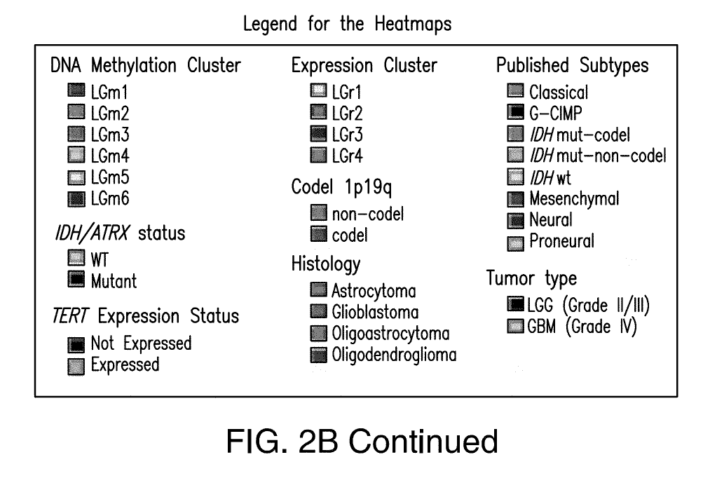

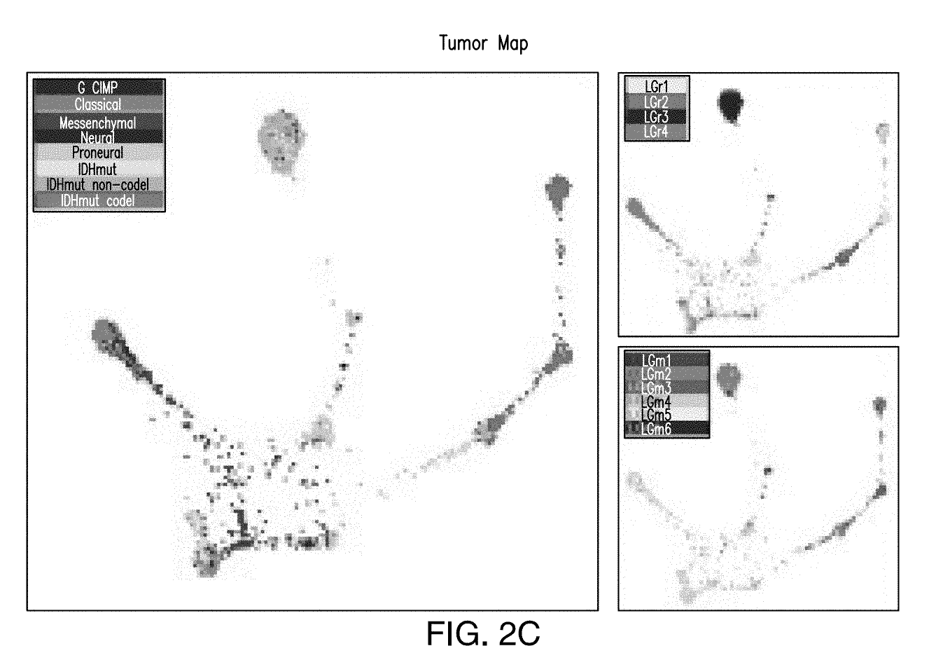

Methods to capture tumor-specific DNA methylation probes were used as recently described (Cancer Genome Atlas Research, 2014b). First probes which had any “NA”-masked data points and probes that were designed for sequences on X and Y chromosomes were removed. CpG sites that are located in high CpG density regions (top 25% of the sites with the highest observed/expected CpG ratio around their 3 kb regions spanning from 1,500 bp upstream to 1,500 bp downstream of the transcription start sites) were selected and CpGs associated with CpG islands were extracted from the UCSC Genome Browser (http://genome.ucsc.edu). To capture cancer-specific DNA hypermethylation events, sites that were methylated (mean β-value ≥0.3) in histologically non-tumor brain tissues (Guintivano et al., 2013) were further eliminated. This selection method reduced the initial 25,978 probes to 1,300 glioma-specific CpG probes, which corresponded to 6.5% of the full available data. However, a clustering analysis can be strongly confounded by the purity of tumor samples. To alleviate the potential influence of variable levels of tumor purity in the sample set on the clustering result, the data was dichotomized using a β-value of >0.3 as a threshold for positive DNA methylation. Then unsupervised hierarchical clustering was performed on 1,300 CpG sites with this threshold that were methylated in at least 10% of the tumors using a binary distance metric for clustering and Ward's method for linkage. The cluster assignments were generated by cutting the resulting dendrogram. The probes were arranged based on the order of unsupervised hierarchical clustering of the dichotomous data using a binary distance metric and Ward's linkage method. Six groups (LGm1-LGm6) shown in FIG. 2A were generated based on the original β-values to visualize 1,300 CpG sites used in the clustering.

The probes included 1300 probes for Pan Glioma Clusters (unsupervised), 1308 IDHmut clusters (unsupervised), 914 IDHwt clusters (unsupervised), 131 probes that define G-CIMP-low, 149 probe signature for IDHmut, 27 probe signature (and EReg) for IDHwt, and 45 probes for the IDHmut EReg.

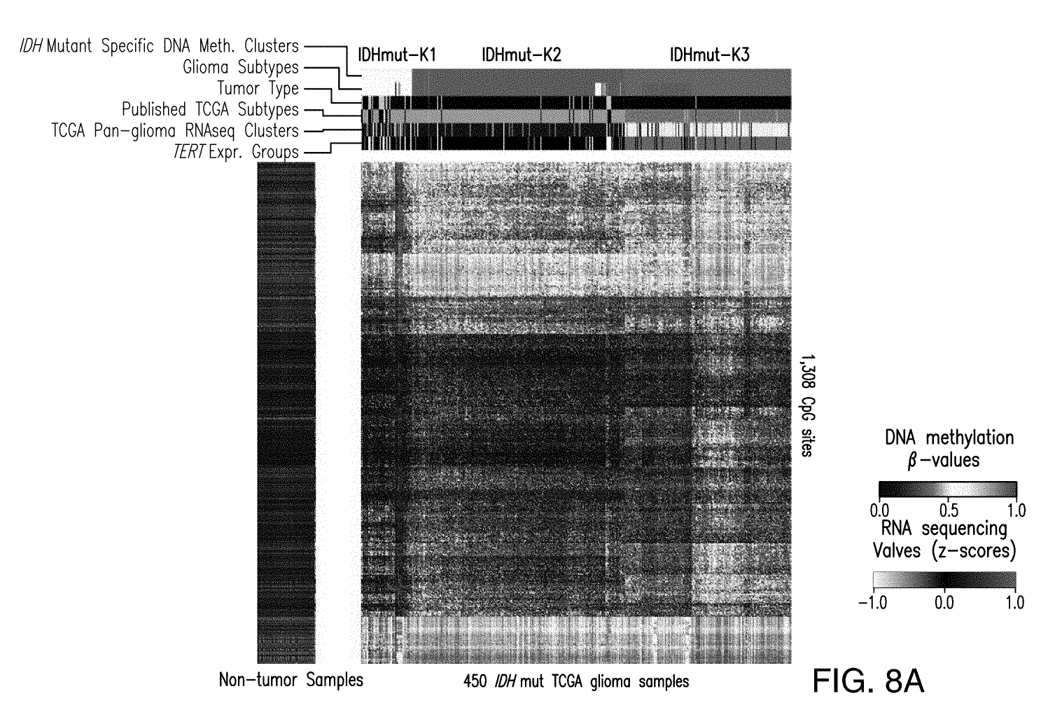

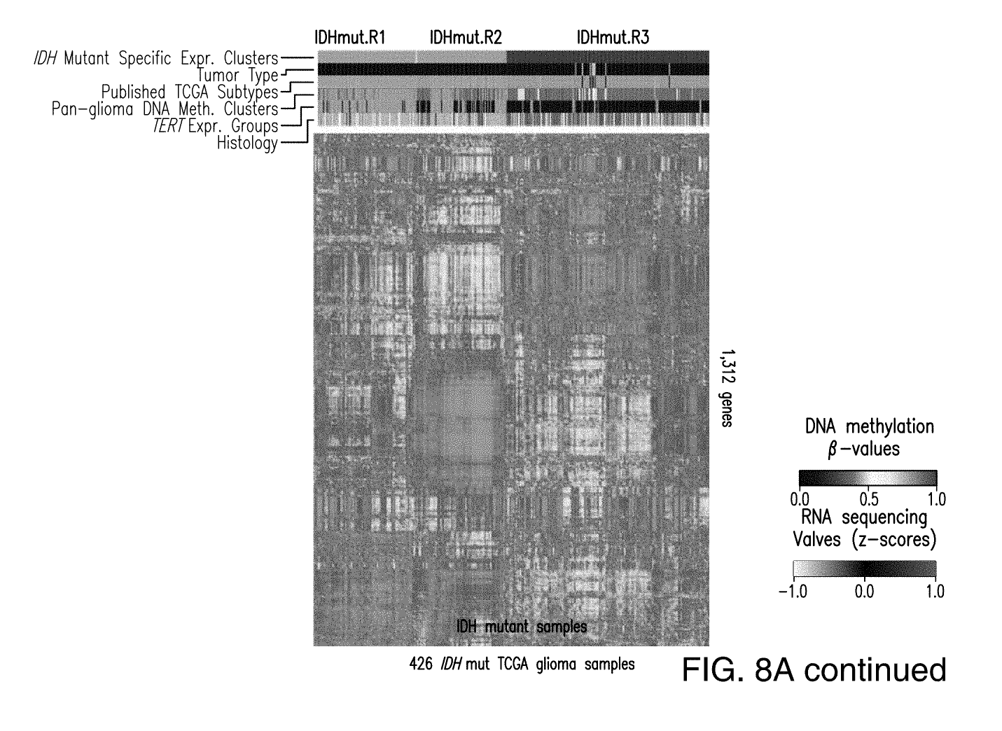

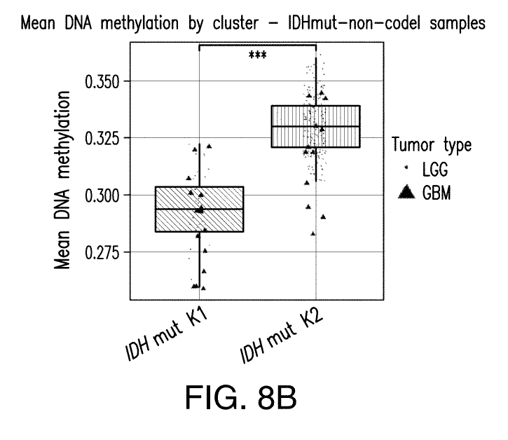

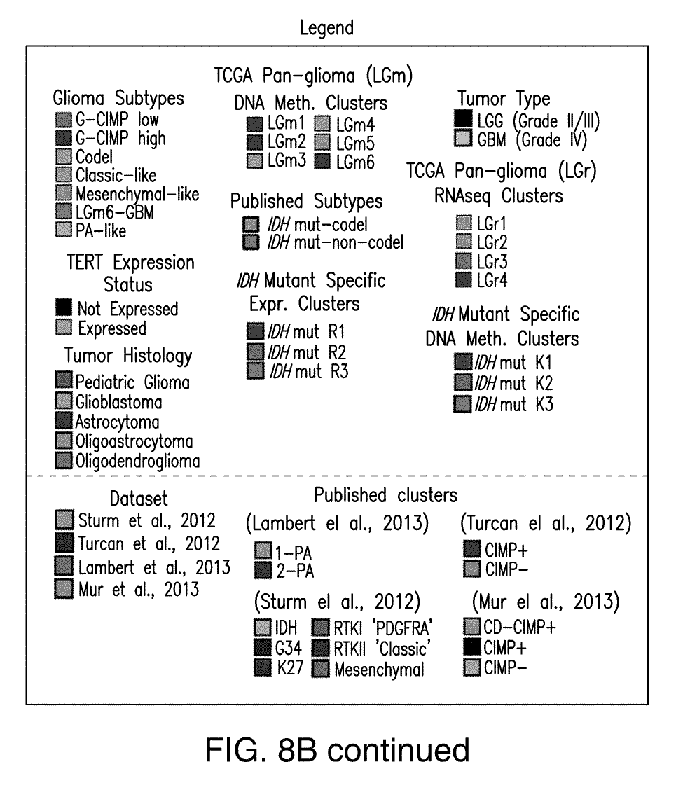

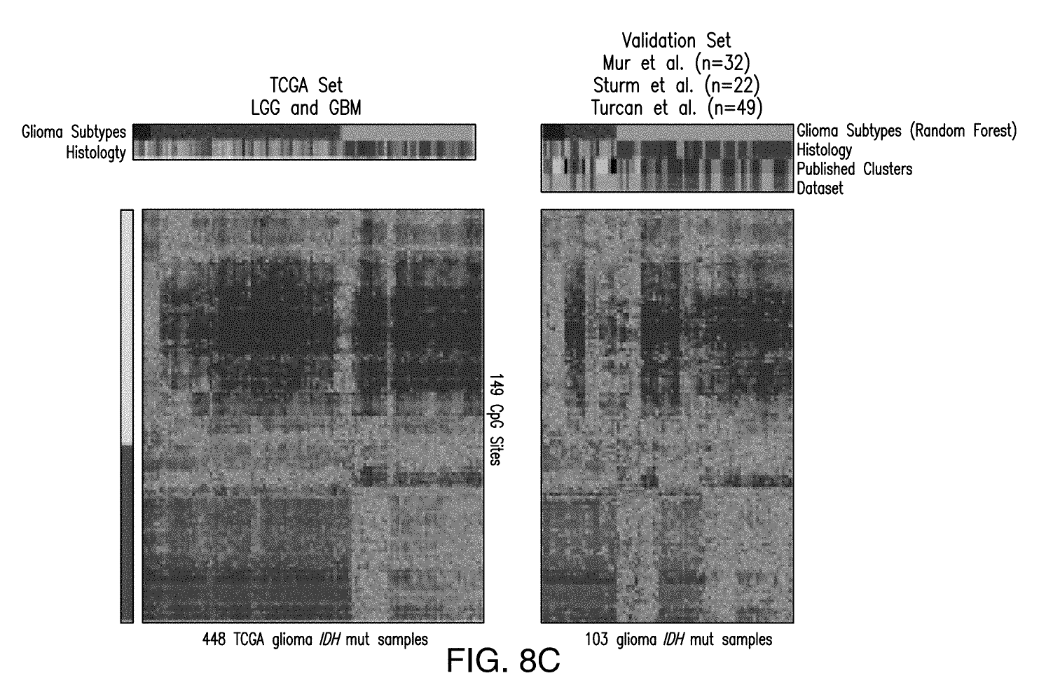

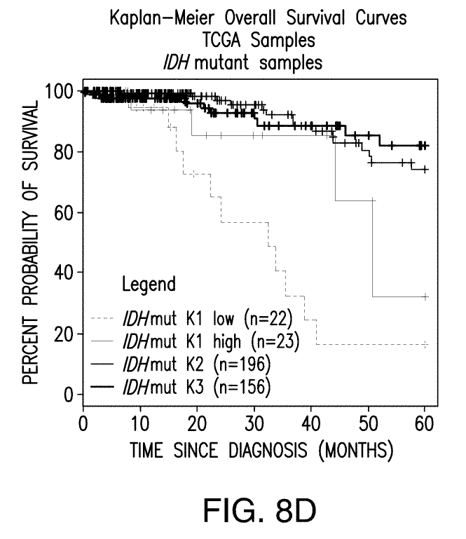

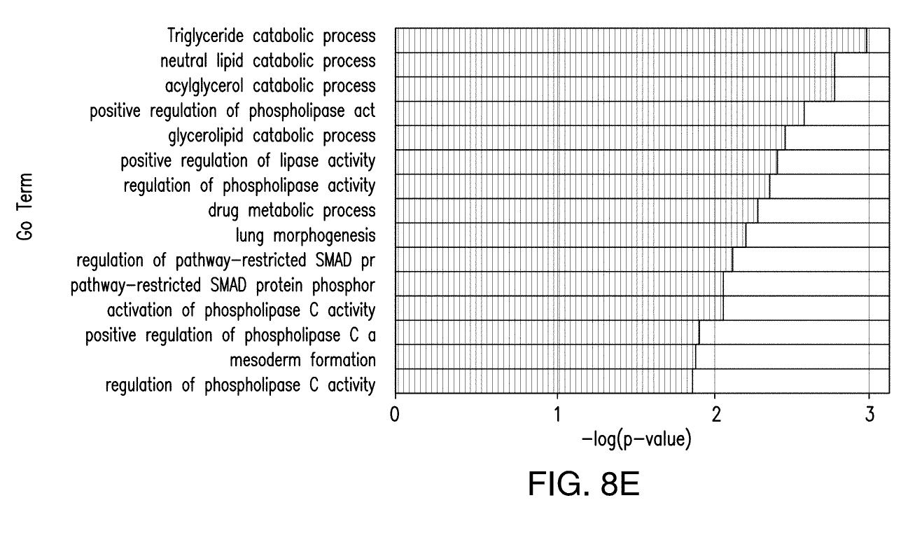

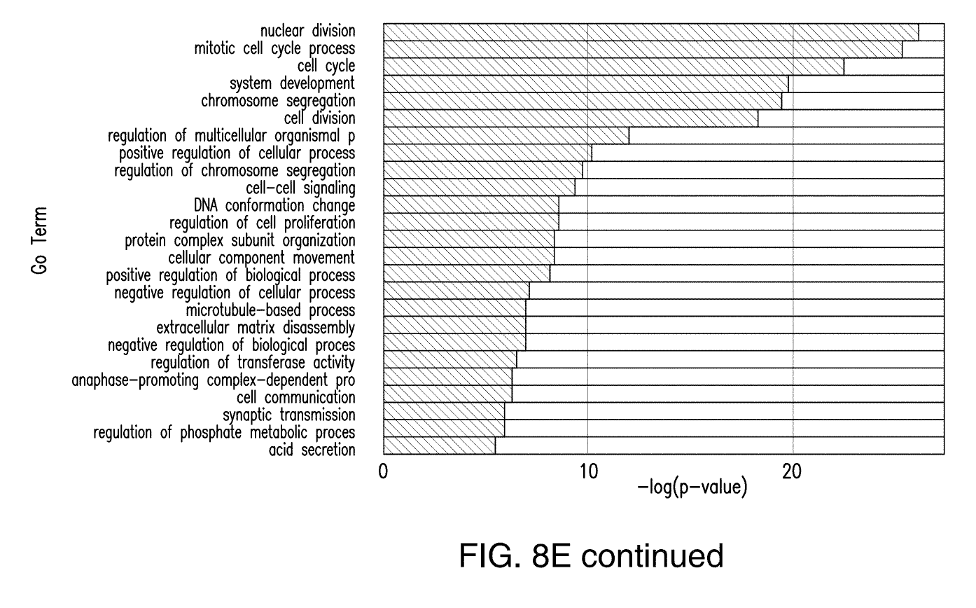

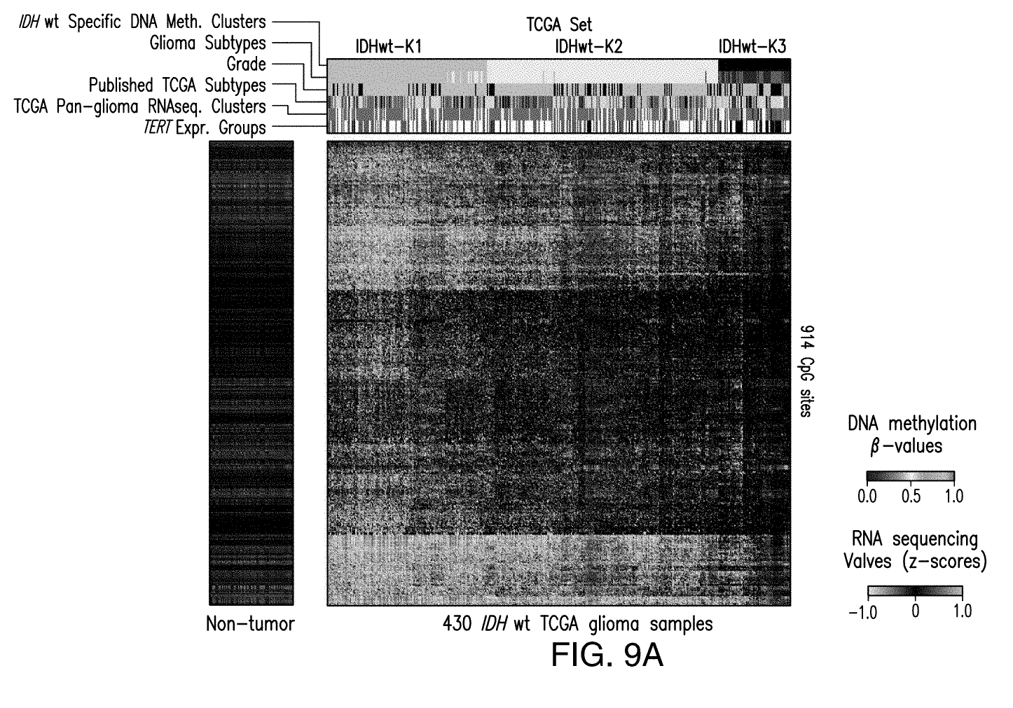

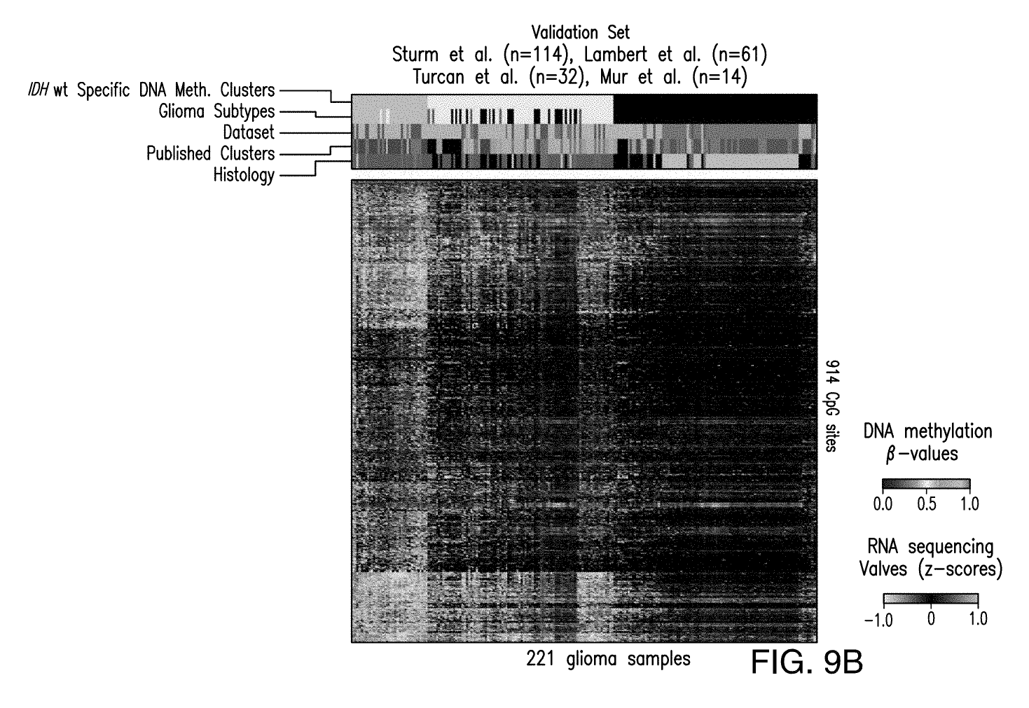

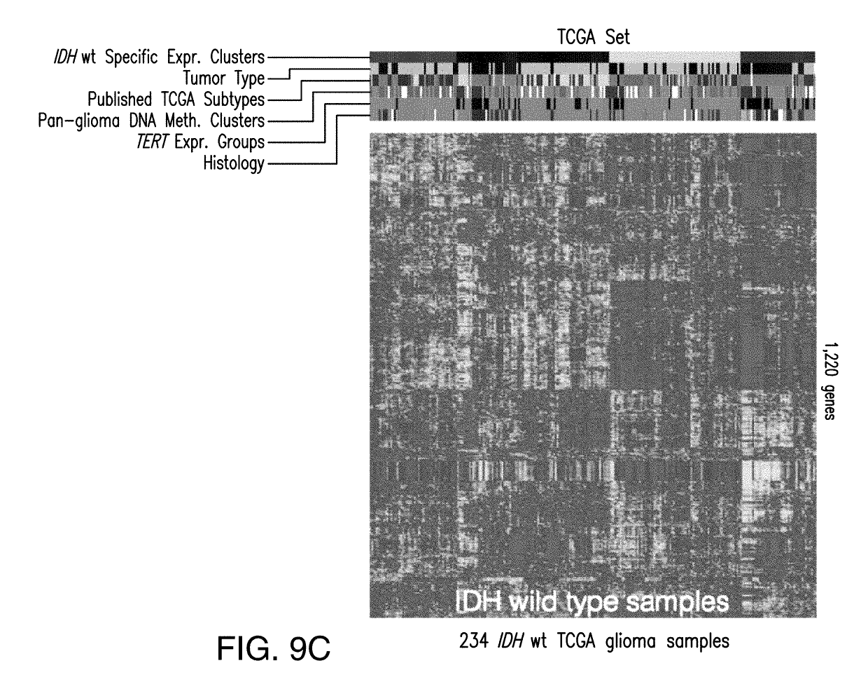

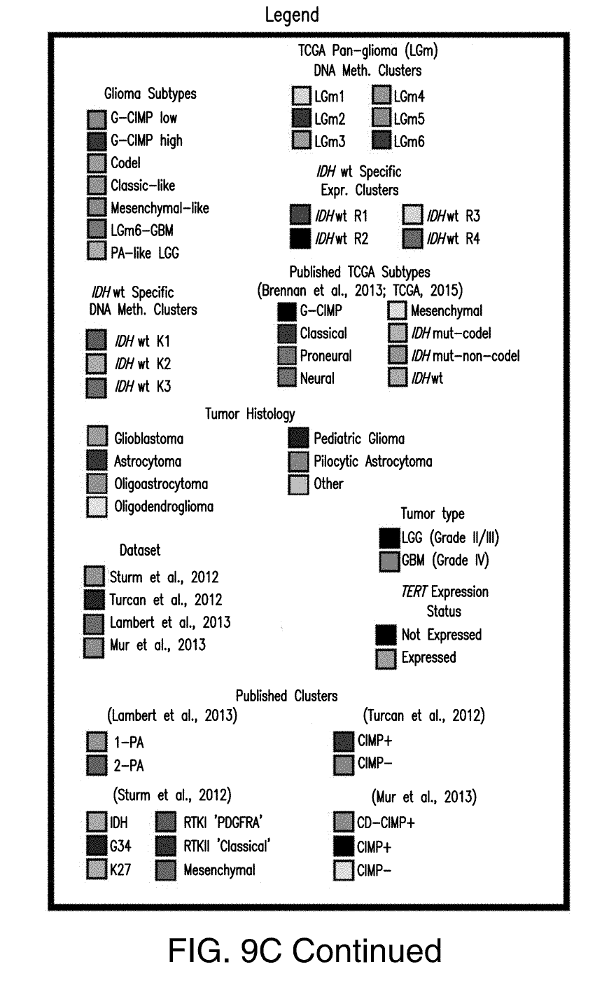

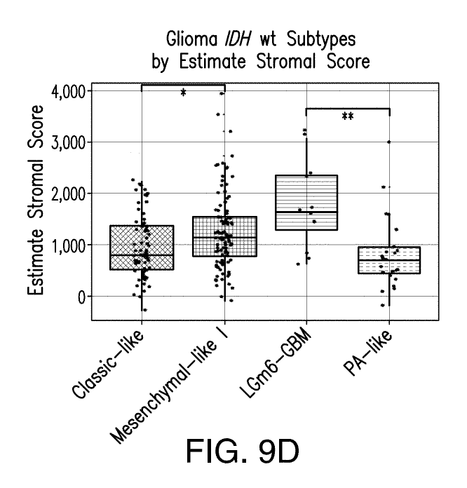

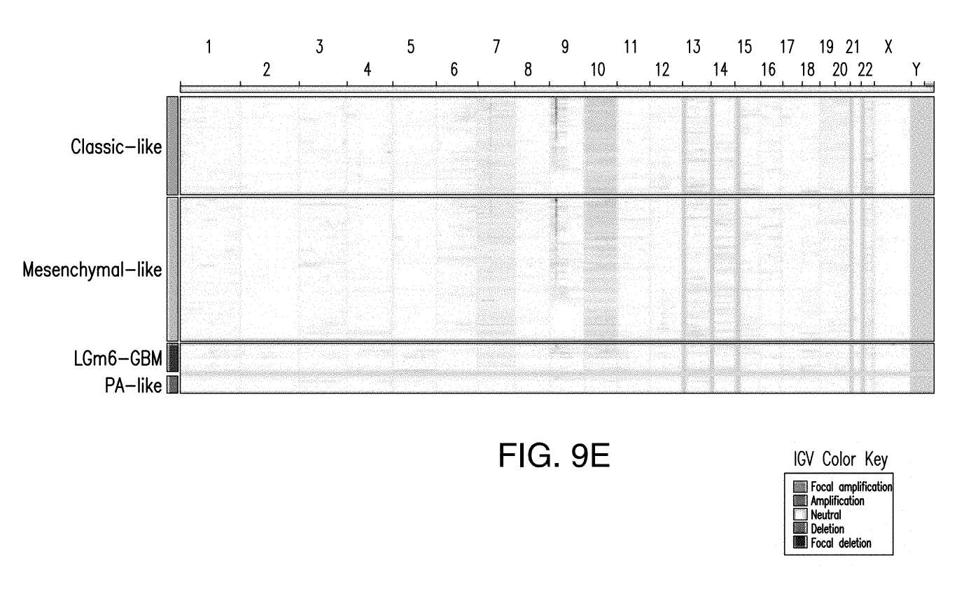

The approach described above to capture tumor-specific DNA methylation probes was used to select glioma-specific CpG probes and perform unsupervised clustering separated by IDH status. 1,308 tumor specific CpG probes for IDH-mutant analysis and three IDHmutant-specific clusters (FIG. 8A) were identified. Likewise, 914 tumor specific CpG probes for IDH-wild type samples and three IDH-wildtype-specific clusters (FIG. 9A) were identified.

In order to classify the newly acquired TCGA samples (not included in the previous studies; LGG=227; GBM=20) into the context of previously published DNA methylation clusters (Brennan et al., 2013; TCGA_Network, 2015), a set of 80% of TCGA samples were randomly selected to train a random forest machine-learning. The performance was then evaluated on the remaining 20% of samples and an accuracy of more than 88% on average was achieved. The new TCGA samples were then tested and classified into the previous DNA methylation clusters.

Supervised Analysis of DNA Methylation

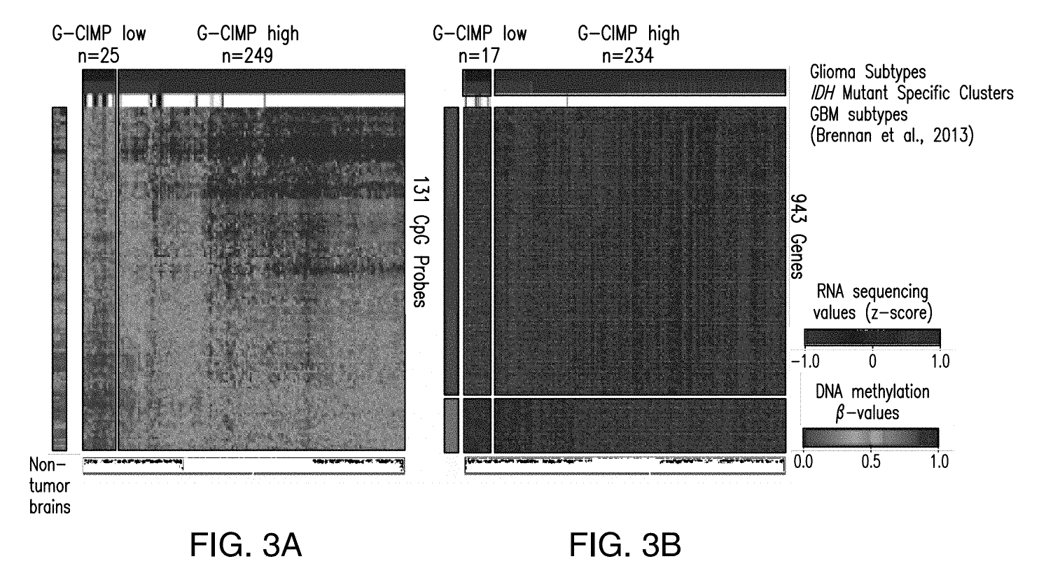

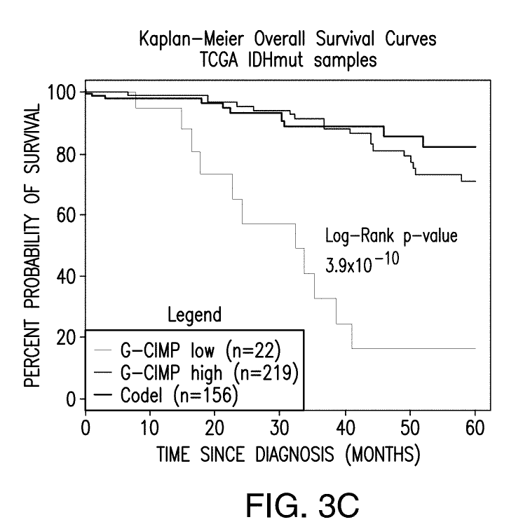

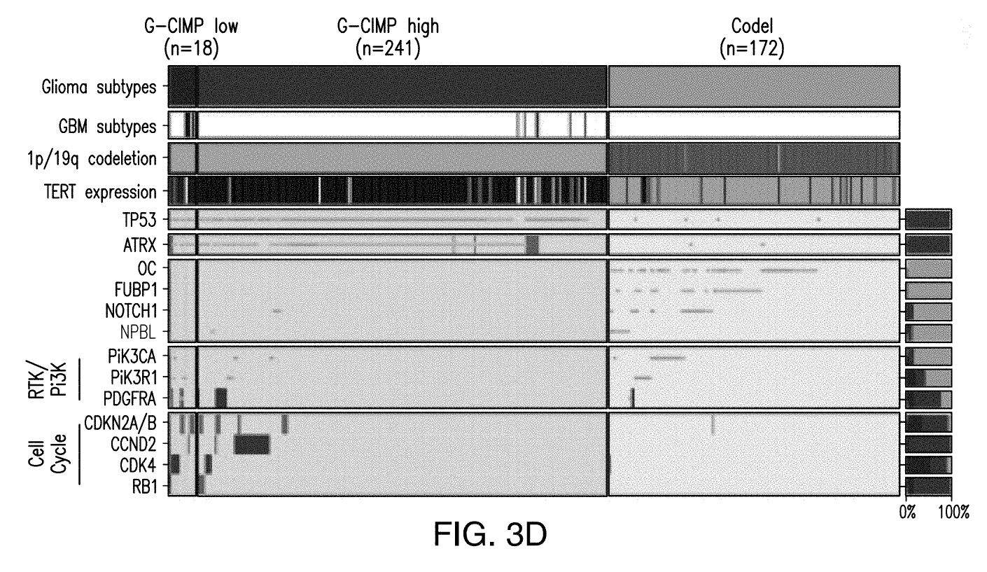

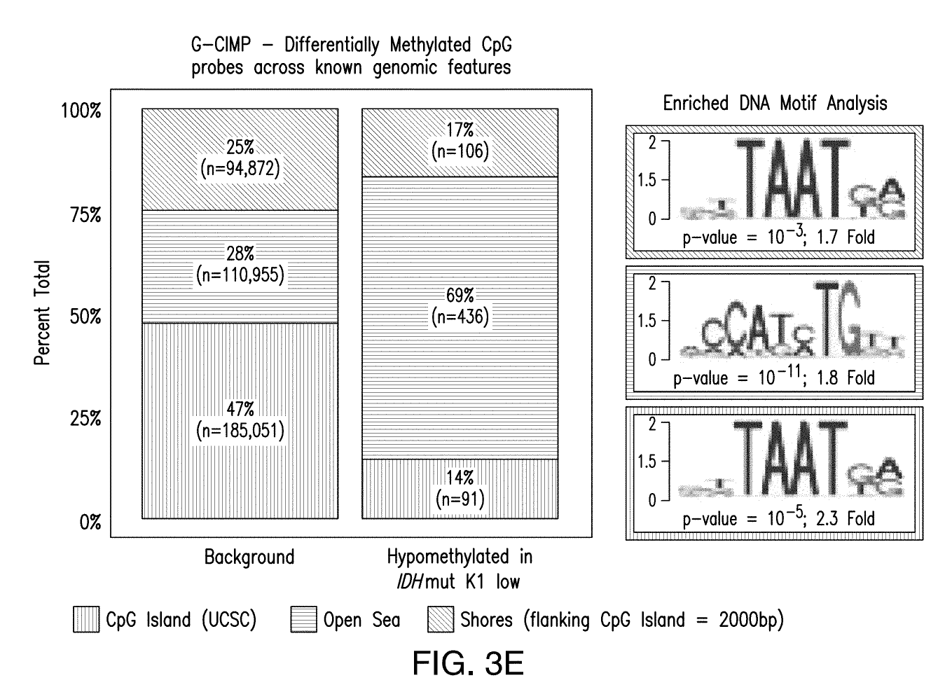

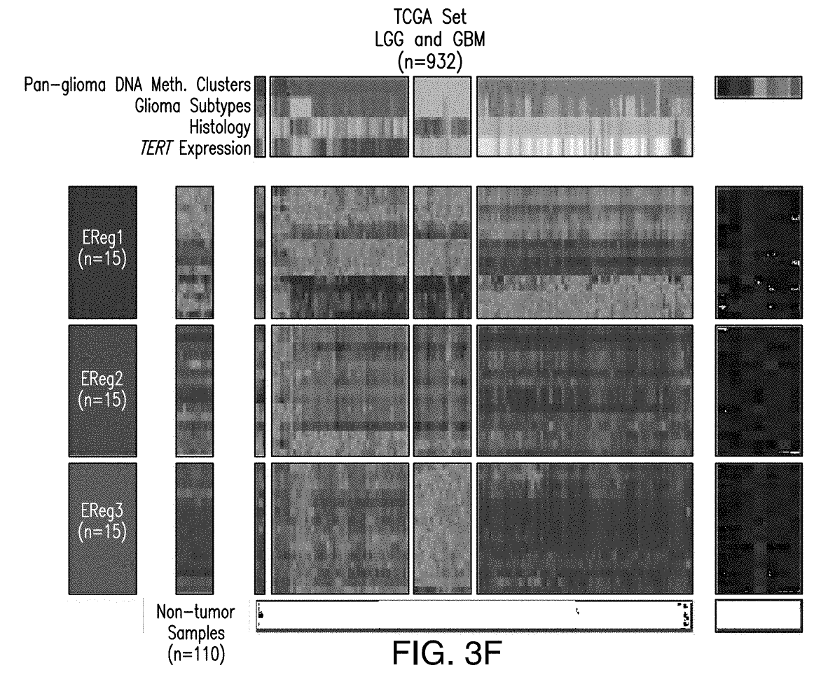

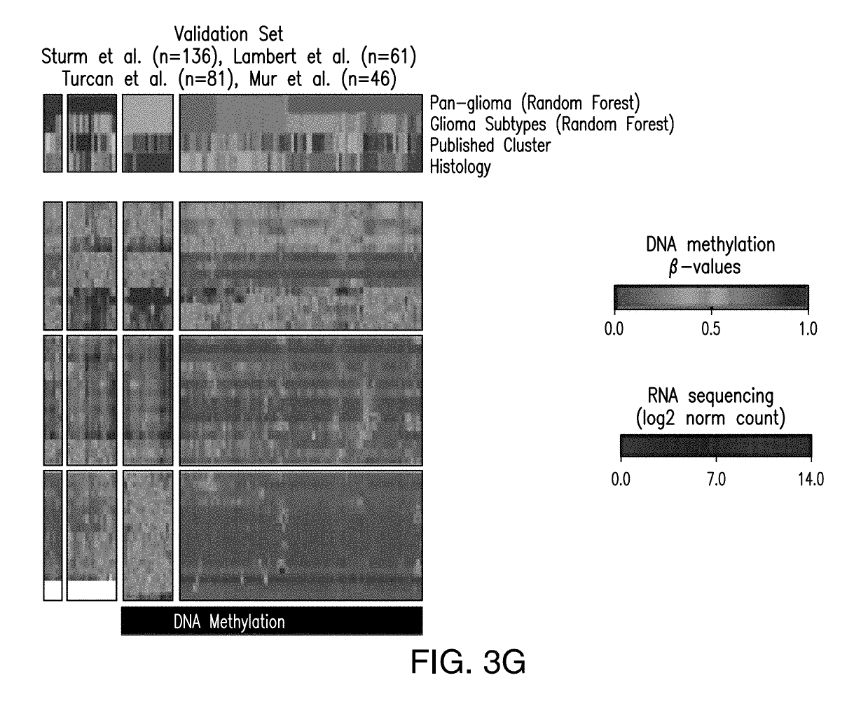

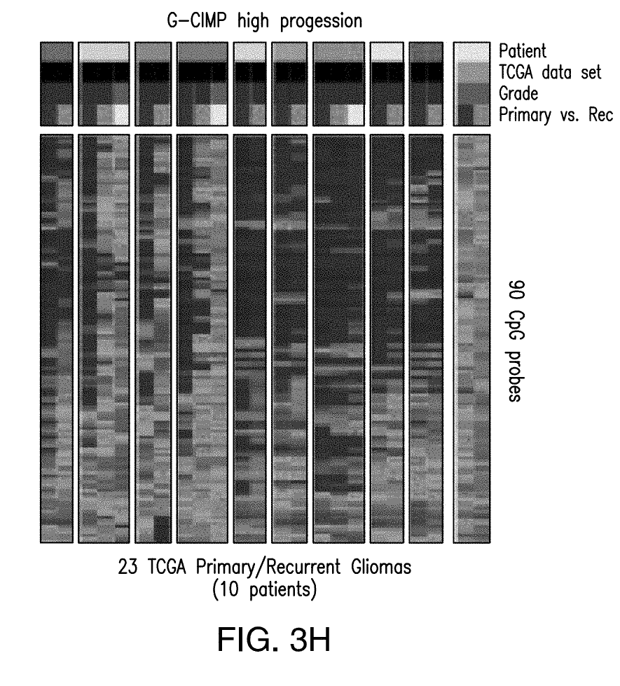

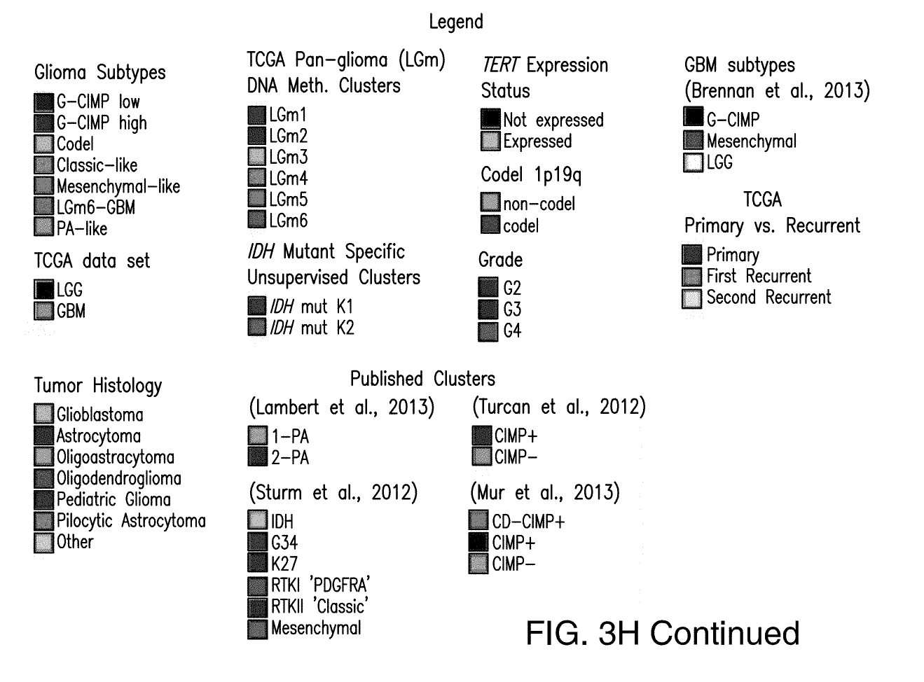

The Wilcoxon test followed by multiple testing using the Benjamini and Hochberg (BH) method was used for false discovery rate estimation (Benjamini and Hochberg, 1995) to identify differentially DNA methylated probes between two groups of interest. The 131 probes presented in FIG. 3A were defined comparing samples from IDHmut-K1 (n=53) to IDHmut-K2 (n=221), using the following criteria: FDR<10e-15, absolute difference in mean methylation beta-value >0.27. The 90 probes presented in FIG. 311 were identified comparing samples from G-CIMP-low (n=25) to G-CIMP-high (n=249), in order to identify probes defining the G-CIMP-low group, using the following criteria: FDR<10e-13, difference in mean methylation beta-value >0.3 and <−0.4. The 149 probes presented in FIG. 3H were a combination of the 90 probes described above with 73 probes identified from the comparison between non-codels (from LGm2, n=210) and codels (from LGm3, n=120), using the following criteria: FDR<10e-30, absolute difference in mean methylation beta-value >0.25, removing probes with NA values.

Identification of Epigenetically Regulated Genes

To increase statistical power, epigenetically regulated genes were evaluated using the Pan-glioma subtypes, which allowed use of the entire TCGA glioma cohort. Tumor samples that had both DNA methylation and RNA-sequencing based gene expression data were selected to do this analysis, resulting in 636 samples (513 LGG and 123 GBM). 110 non-tumor TCGA samples from 11 different tissues were also randomly selected and profiled using the same platforms. Each DNA methylation probe was mapped to the nearest UCSC gene, and after merging the DNA methylation and gene expression data, a total of 19,530 pairs of DNA methylation and gene expression probes were retained. The tumor samples were organized as either methylated (β>=0.3) or unmethylated (β<0.3) for each probe. The pair of DNA methylation and gene expression probes for which the mean expression in the methylated group was lower than 1.28 standard deviation (bottom 10%) of the mean expression in the unmethylated group, and in which >80% of the samples in the methylated group had expression levels lower than the mean expression in the unmethylated group were selected. Each tumor sample was labeled as epigenetically silenced for a specific probe/gene pair if: it belonged to the methylated group and the gene expression level was lower than the mean of the unmethylated group silenced (Cancer Genome Atlas Research, 2014a), resulting in 3,806 probes/genes identified as epigenetically regulated.

A Fisher test was used to detect if these 3,806 pairs were enriched in a DNA methylation cluster. For each probe, tumor samples labeled as methylated and downregulated by cluster, while non-tumor samples labeled as unmethylated and upregulated, were counted and arranged into a contingency table for a Fisher test, using 50% as a cutoff. p-value was calculated for each probe/gene pair and then was adjusted for multiple testing using the BH method for false discovery rate estimation (Benjamini and Hochberg, 1995). This analysis identified 3 Epigenetically Regulated groups (EReg): EReg2 with 233 genes enriched in LGm2 (resembling G-CIMP high), EReg3 with 15 genes enriched in LGm3 (resembling Codels) and EReg4 with 14 genes enriched in LGm4 (resembling Classic-like) and 1 gene enriched in LGm5 (resembling Mesenchymal-like). Since LGm1 (enriched for G-CIMP-low) and LGm6 (comprising LGm6-GBM and PA-like) are heterogeneous clusters, a different approach was applied in order to identify epigenetically regulated genes for these groups. For EReg1, the DNA methylation and gene expression levels for G-CIMP-low samples (n=25) with GCIMP-high samples (randomly selected 50 samples out of 249) were compared and those probes/genes with Wilcoxon BH adjusted p-value less than 1e-10, methylation difference greater than 0.25 and RNA expression log Fold Change greater than 0.85 were selected, resulting in 15 epigenetically regulated genes enriched in G-CIMP-low. For EReg5, the DNA methylation levels for LGm6 samples (n=77) were compared with a subset of randomly selected samples from the 855 remaining TCGA glioma samples (n=140) and those probes with Wilcoxon BH adjusted p-value less than 1e-21 and methylation difference greater than 0.33 were selected, resulting in 12 epigenetically regulated genes enriched in LGm6.

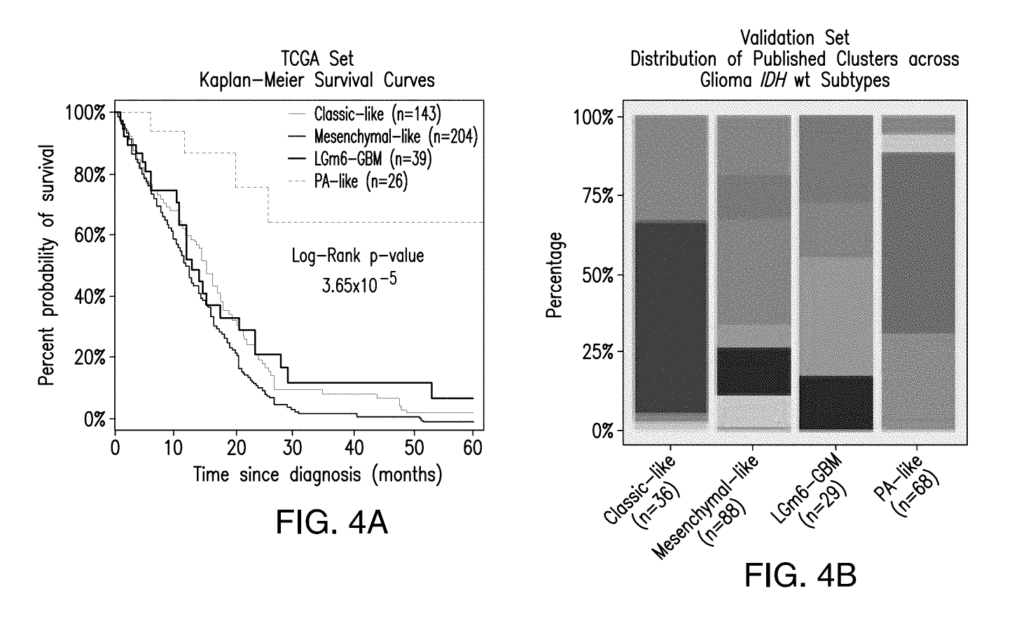

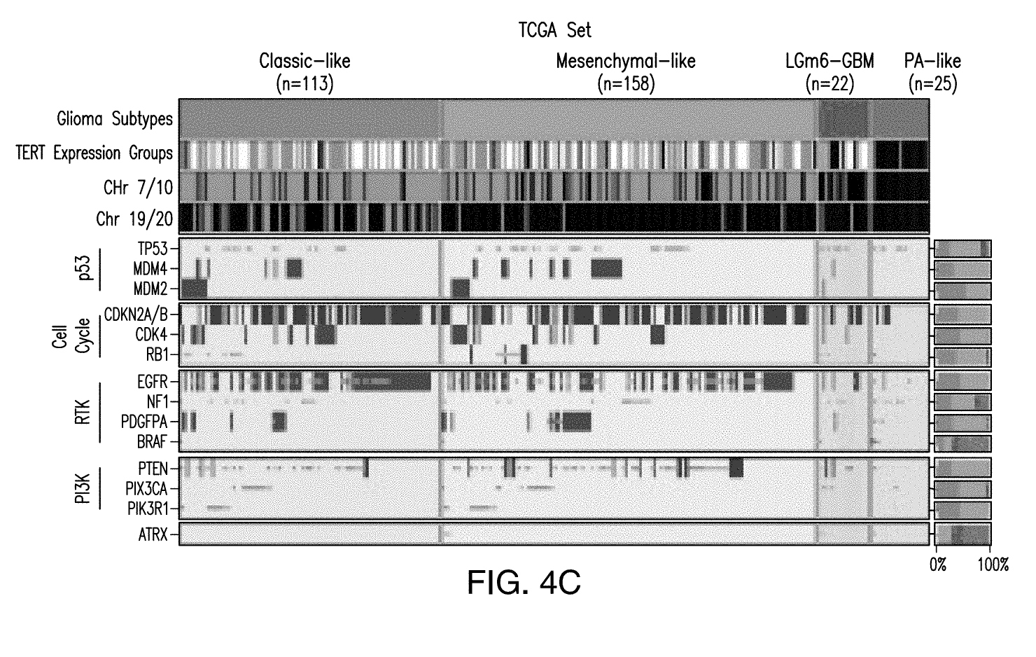

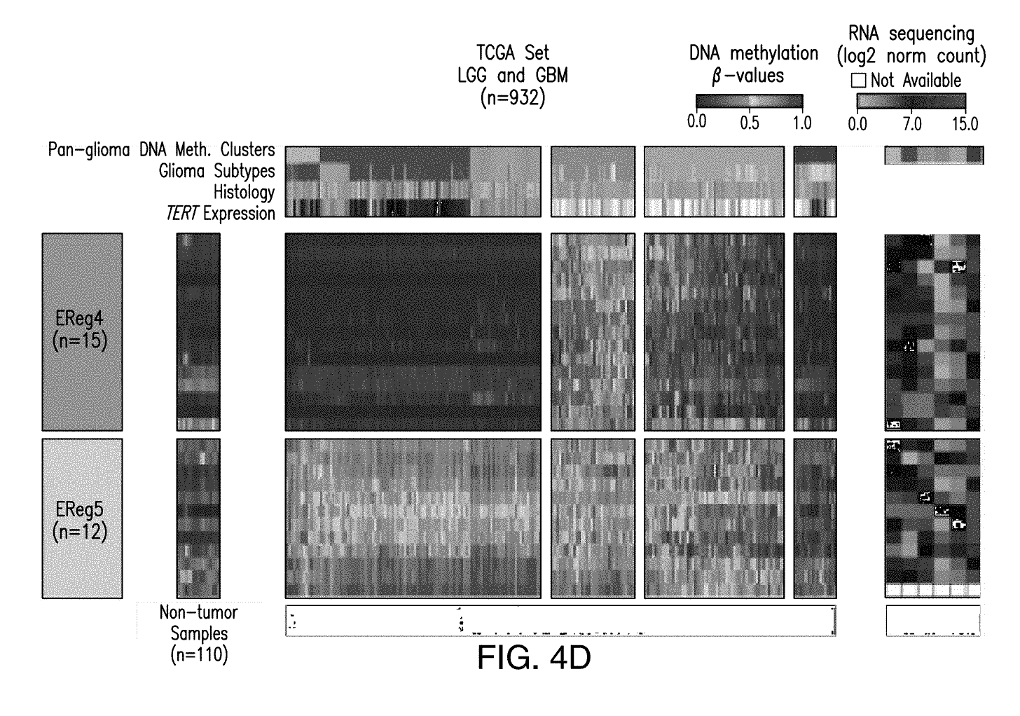

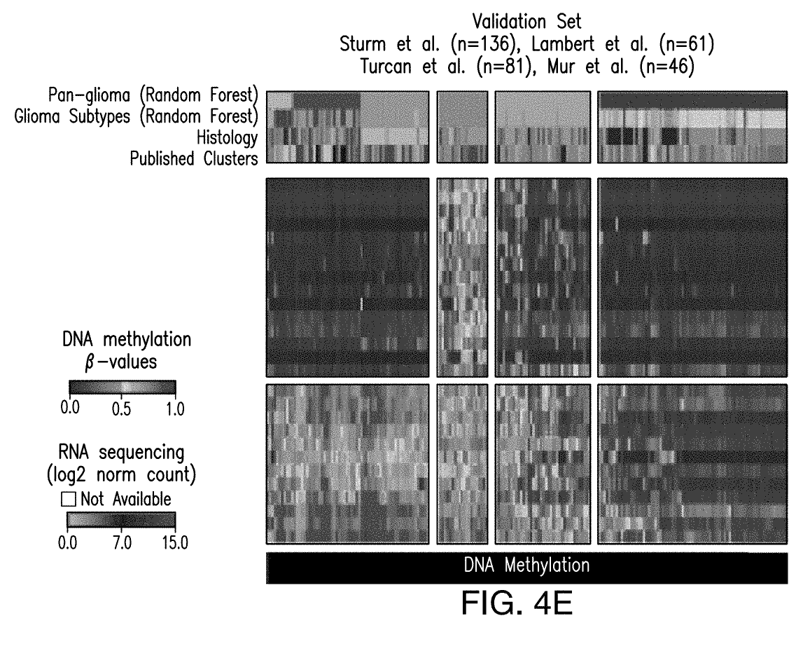

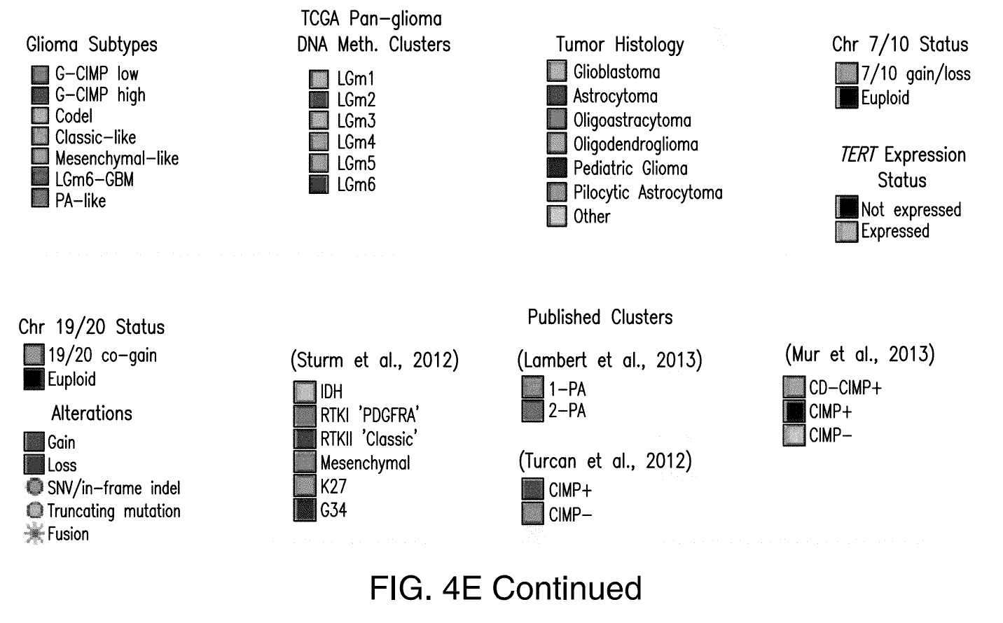

To validate the EReg genes in order to confirm the existence of these signatures in an independent, non-TCGA data, 4 different and publicly available datasets (Lambert et al., 2013; Mur et al., 2013; Sturm et al., 2012; Turcan et al., 2012), including 324 samples with distinct histology and clinical attributes were used. These samples included adult, pediatric gliomas of both low and high grade, reported with codel, IDH status and G-CIMP status. The independent data set included a pool of 61 pilocytic astrocytomas defined as grade I gliomas (Lambert et al., 2013). In order to classify the additional non-TCGA gliomas into LGm clusters, a random set of 80% TCGA samples were selected to train a random forest machine-learning model and the performance was evaluated on the remaining 20%. Given the high specificity and sensitivity of this model (accuracy >88% on average), the LGm cluster prediction model was tested on the additional non-TCGA samples using the random forest method. Data were visualized using the same 45 pairs of CpG probes/genes that define the epigenetically regulated genes for IDH mutant samples (FIG. 3F) and the same 27 pairs of CpG probes/genes that define the epigenetically regulated genes for IDH wild type samples (FIG. 4D). Applying a similar ordering in the validation set and accounting for differences in sample size, the five EReg groups both for IDH mutant samples (FIG. 3G) and IDH wild type samples (FIG. 4E) were recapitulated in molecular level.

The same random forest machine learning model approach was used for the IDH-mutant samples (using the 1,308 IDH-mutant tumor specific CpG probes) and for the IDH-wildtype samples (using the 914 IDH-wildtype tumor specific CpG probes), separately. The models were then tested in the IDH-mutant and IDH-wildtype samples from the validation set (Lambert et al., 2013; Mur et al., 2013; Sturm et al., 2012; Turcan et al., 2012) (Figure S4B).

Patient Centric Table (DNA Methylation)

To generate DNA methylation calls for each sample per gene per overlapping platforms (HM27, HM450), first multiple CpGs were collapsed to one representative gene. Using the associated gene expression data (organized as one gene—one expression value per sample), the samples and CpG probes with gene expression data for each platform were merged. Next, the Spearman correlation (p) across all samples for all CpG probes for each gene to one gene expression value was calculated. For multiple CpGs for each annotated gene promoter, one CpG probe with the lowest correlation rho value to the associated gene expression profile was selected to capture the most biologically representative event (epigenetic silencing). This effectively reduced the number of CpG probes from N:1 to 1:1. The data set was then reduced down to 636 samples×19,486 CpG:Gene.

Next, discrete categories were assigned based on the Spearman correlation rho value according to the following criteria:

1. Strongly negatively correlated (SNC) when ρ value is less than 0.5;

2. Weakly negatively correlated (WNC) when ρ value is between 0.5 and 0.25;

3. No negative correlation (NNC) when p value is greater than 0.25.

Next, samples were assigned to either the 10th (T10 or N10) or 90th (T90 or N90) percentile based on the observed beta-value across tumor samples (T) and normal samples (N). For the normal samples, 110 non-tumor TCGA samples from 11 different tissues previously described were used. Labels were assigned for each gene per platform per tissue type (tumor and normal) according to the following rules:

1. If percentile 90<0.25, assigned as CUN or CUT (constitutively unmethylated in normal or tumor);

2. If percentile 10>0.75, assigned as CMN or CMT (constitutively methylated in normal or tumor);

3. If percentile 10>0.25 and percentile 90<0.75, assigned as IMN or IMT (intermediate methylated in normal or tumor);

4. If not in any of the above categories, assinged as VMN or VMT (variably methylated in normal or tumor).

Next a ‘call’ and a confidence ‘score’ was assigned for each possible combinations (48) [3 (SNC, WNC, NNC)×4 (CUN, CMN, VMN, IMN)×4 (CUT, CMT, VMT, IMT)]. The following relationship for each call and score was created based on an interpretation of the most informative epigenetic event (e.g. promoter DNA hypermethylation and low expression).

The methylation calls are as follows:

MG: Methylation gain compared to normal

ML: Methylation loss compared to normal

MT: Methylated in tumor

UT: Unmethylated in tumor

ES: Epigenetically silenced

UC: Unable to make call

Methylation class confidence scores varied from 0 (no call) to 4 (high confidence).

Homer De Novo Motif Searches

De novo Motif discovery was performed using HOMER (script v4.4 (Aug. 25, 2014)), an algorithm previously described (Heinz et al., 2010). Briefly, differentially methylated probes were classified according to genomic location into CpG island, CpG shores, and open seas as follow: CpG islands were defined based on UCSC annotation and as per the criteria previously described (GardinerGarden and Frommer, 1987; Takai and Jones, 2002). Coverage of CpG island regions was further enhanced by including the 2 kb regions flanking CpG island, referred to here as CpG shores. CpGs isolated in the genome were defined as open seas. Probes mapped to each region were used to performed de novo motif analysis using HOMER (HOMER perl script ‘findMotifsGenome.pl’). To increase sensitivity of the method, up to two mismatches were allowed in each oligonucleotide sequence and distributions of CpG content in ‘target’ and ‘background’ sequences were selectively weighted to equalize the distributions of CpG content in both sets.

Reverse Phase Protein Array (RPPA) Data Processing

Protein was extracted using RPPA lysis buffer (1% Triton X-100, 50 mmol/L Hepes (pH 7.4), 150 mmol/L NaCl, 1.5 mmol/L MgCl2, 1 mmol/L EGTA, 100 mmol/L NaF, 10 mmol/L NaPPi, 10% glycerol, 1 mmol/L phenylmethylsulfonyl fluoride, 1 mmol/L Na3VO4, and aprotinin 10 ug/mL) from human tumors and RPPA was performed as described previously (Coombes, 2011; Hennessy et al., 2007; Hu et al., 2007; Liang et al., 2007; Tibes et al., 2006). Lysis buffer was used to lyse frozen tumors by Precellys homogenization. Tumor lysates were adjusted to 1 μg/μL concentration as assessed by bicinchoninic acid assay (BCA) and boiled with 1% SDS. Tumor lysates were manually serial diluted in two-fold of 5 dilutions with lysis buffer. An Aushon Biosystems 2470 arrayer (Burlington, Mass.) printed 1,056 samples on nitrocellulose-coated slides (Grace Bio-Labs). Slides were probed with 196 validated primary antibodies (Cancer Genome Atlas Research, 2015) followed by corresponding secondary antibodies (Goat anti-Rabbit IgG, Goat anti-Mouse IgG or Rabbit antiGoat IgG). Signal was captured using a DakoCytomation-catalyzed system and DAB colorimetric reaction. Slides were scanned in a CanoScan 9000F. Spot intensities were analyzed and quantified using Array-Pro Analyzer (Media Cybernetics Washington D.C.) to generate spot signal intensities (Level 1 data). The software SuperCurveGUI (Coombes, 2011; Hu et al., 2007), was used to estimate the EC50 values of the proteins in each dilution series (in log 2 scale). Briefly, a fitted curve (“supercurve”) was plotted with the signal intensities on the Y-axis and the relative log 2 concentration of each protein on the X axis using the non-parametric, monotone increasing B-spline model (Tibes et al., 2006). During the process, the raw spot intensity data were adjusted to correct spatial bias before model fitting. A QC metric (Coombes, 2011) was returned for each slide to help determine the quality of the slide: if the score is less than 0.8 on a 0-1 scale, the slide was dropped. In most cases, the staining was repeated to obtain a high quality score. If more than one slide was stained for an antibody, the slide with the highest QC score was used for analysis (Level 2 data). Protein measurements were corrected for loading as described (Coombes, 2011; Gonzalez-Angulo et al., 2011; Hu et al., 2007) using median centering across antibodies (level 3 data). In total, 196 antibodies and 473 samples were used. Final selection of antibodies was also driven by the availability of high quality antibodies that consistently pass a strict validation process as previously described (Hennessy et al., 2010). These antibodies were assessed for specificity, quantification and sensitivity (dynamic range) in their application for protein extracts from cultured cells or tumor tissue. Antibodies were labeled as validated and use with caution based on degree of validation by criteria previously described (Hennessy et al., 2010).

Two RPPA arrays were quantitated and processed (including normalization and load controlling) as described previously, using MicroVigene (VigeneTech, Inc., Carlisle, Mass.) and the R package SuperCurve (version-1.3), (Hu et al., 2007; Tibes et al., 2006). Raw data (level 1), SuperCurve nonparameteric model fitting on a single array (level 2), and loading corrected data (level 3) were deposited at the DCC.

Reverse Phase Protein Array (RPPA) Data Normalization

Median centering across all the antibodies was performed for each sample to correct for sample loading differences. Those differences arise because protein concentrations are not uniformly distributed per unit volume. That may be due to several factors, such as differences in protein concentrations of large and small cells, differences in the amount of proteins per cell, or heterogeneity of the cells comprising the samples. By observing the expression levels across many different proteins in a sample, one can estimate differences in the total amount of protein in that sample vs. other samples. Subtracting the median protein expression level forces the median value to become zero, allowing one to compare protein expressions across samples.

Surprisingly, processing similar sets of samples on different slides of the same antibody may result in datasets that have very different means and variances. Neely et al. (2009) processed clinically similar ALL samples in two batches and observed differences in their protein data distributions. There were additive and multiplicative effects in the data that could not be accounted by biological or sample loading differences. Similar effects were observed when comparing the two batches of GBM and LGG tumor protein expression data. A new algorithm, replicates-based normalization (RBN), was therefore developed using replicate samples run across multiple batches to adjust the data for batch effects. The underlying hypothesis is that any observed variation between replicates in different batches is primarily due to linear batch effects plus a component due to random noise. Given a sufficiently large number of replicates, the random noise is expected to cancel out (mean=zero by definition). Remaining differences are treated as systematic batch effects. One can compute those effects for each antibody and subtract them.

Many samples were run in both batches. One batch was arbitrarily designated the “anchor” batch and was to remain unchanged. The means and standard deviations of the common samples in the anchor batch, as well as the other batch were computed. The difference between the means of each antibody in the two batches and the ratio of the standard deviations provided an estimate of the systematic effects between the batches for that antibody (both location-wise and scale-wise). Each data point in the non-anchor batch was adjusted by subtracting the difference in means and multiplying by the inverse ratio of the standard deviations to cancel out those systematic differences. This normalization procedure significantly reduced technical effects, thereby allowing the datasets from different batches the merged.

Clustering

Consensus clustering was used to cluster the samples in an unsupervised way, with Pearson correlation as the distance metric and Ward as the linkage algorithm. A total of 473 samples and 196 antibodies were used in the analysis. Two clusters were observed that largely corresponded with tumor type (FIG. 8E), however, there were a few notable exceptions. Whereas only one GBM sample clustered with the LGG samples, twenty-six LGG samples were found to cluster with the GBM samples. Seventeen of those twenty-six samples had no mutations in IDH1/2, similar to the GBM samples. Furthermore, compared to the LGG-like cluster, the GBM-like cluster had elevated expression of IGFBP2, fibronectin, PAIL HSP70, EGFR, phosphoEGFR, phosphoAKT, Cyclin B1, Caveolin, Collagen VI, Annexin1 and ASNS, whereas it had low expression of PKC (alpha, beta and delta), PTEN, BRAF, and phosphoP70S6K.

Regulome Explorer Feature Matrix

Associations among the diverse clinical and molecular data were identified through construction of a “feature matrix” (FM) by integrating values from all data types. Each column in the FM represents one of the 1123 tumor samples. Each row in the FM represents a single clinical, sample or molecular data element (mRNA expression levels, microRNA expression levels, protein levels (RPPA), copy number alterations, DNA methylation levels and somatic mutations), and the individual data values may be numerical (continuous or discrete) or categorical, as appropriate. Missing values are indicated within the FM by “NA”, and the number of non-NA data values varies significantly across the different data types (rows). Data were retrieved from the DCC on Nov. 18, 2015 and further processed as follows.

Clinical and sample data (633 features): DCC clinical and sample data were processed into a matrix.

Cluster Assignments:

Cluster memberships resulting from unsupervised clustering for each of the individual molecular data types: SCNA, RNAseq, DNA methylation, and RPPA were incorporated into the FM. Mutation rates and categories were included in the FM as well. Molecular datasets include Gene expression (15,401 features): Gene level RSEM values from RNA-seq were log 2 transformed, and filtered to remove low-variability genes (bottom 25% removed, based on interdecile range). MicroRNA expression (692 features): The summed and normalized microRNA quantification files were log 2 transformed, and filtered to remove low variability microRNAs (bottom 25% based on zero-count).

Somatic Copy Number Alterations:

Copy number and focal copy number changes were obtained for peaks identified by GISTIC as described above (6318 features). DNA methylation (19,727 features): Probe specific level-3 β-values were obtained as described above (Supplement). The analysis began with probes common between the two methylation platforms, and then the bottom 25% based on interdecile range were removed.

Somatic mutations (2842): A mutations annotation file was used to generate a binary indicator vector indicating whether a particular non-silent mutation is present in a specific sample. Mutation features found in fewer than two tumor samples were removed. Overall, the gbm_lgg feature matrix has 45839 features (inclusive of the above mentioned analysis platforms) for all the 1122 patients (data freeze list) resulting in 51477197 matrix elements (48501×38), with approximately 89% non-NA elements (197478 out of 1843038).

All-by-all Pairwise Associations

Statistical association among the diverse data elements in this study was evaluated by comparing pairs of columns in the feature matrix. Hypothesis testing was performed by testing against null models for absence of association, yielding a p-value. P-values for the association between and among clinical and molecular data elements were computed according to the nature of the data levels for each pair: discrete vs. discrete (Fisher's exact test); discrete vs. continuous (ANOVA F-test, equivalently t-test for binary vs. continuous) or continuous vs. continuous (F-test). Ranked data values were used in each case. To account for multipletesting bias, the p-value was adjusted using the Bonferroni correction.

### Example 1: Patient Cohort Characteristics

The patient cohorts used in these examples were characterized. The TCGA LGG and GBM cohorts consisted of 516 and 606 patients, respectively. Independent analysis of the GBM dataset was previously described, as was analysis of 290 LGG samples (Brennan et al., 2013, Cancer Genome Atlas Research Network et al., 2015). 226 LGG samples were added to the current cohort (Table 1). Clinical data, including age, tumor grade, tumor histology, and survival, were available for 93% (1,046/1,122) of cases. The majority of samples were grade IV tumors (n=590, 56%), whereas 216 (21%) and 241 (23%) were grade II and III tumors, respectively. Similarly, 590 (56%) samples were classified as GBM, 174 (17%) as oligodendroglioma, 169 (16%) as astrocytoma, and 114 (11%) as oligoastrocytoma.

Among the data sources considered were gene expression (n=1,045), DNA copy number (n=1,084), DNA methylation (n=932), exome sequencing (n=820), and protein expression (n=473). Multiple and overlapping characterization assays were employed.

TABLE 1
Clinical Characteristics of the Sample Set Arranged by IDH and
1p/19q Co-deletion Status

| | | IDH mut - | IDH mut - | |
|---|---|---|---|---|
| | IDH Wt (n = | non-codel (n = | codel (n = | Unknown (n = |
| Feature | 520) | 283) | 171) | 148) |
| Clinical | | | | |
| Histology (n) | | | | |
| Astrocytoma | 52 (10.0%) | 112 (39.6%) | 4 (2.3%) | 1 (0.7%) |
| Glioblastoma | 419 (80.6%) | 32 (11.3%) | 2 (1.2%) | 137 (92.6%) |
| Oligoastrocytoma | 15 (2.9%) | 69 (24.4%) | 30 (17.5%) | 0 (0%) |
| Oligodendroglioma | 19 (3.7%) | 37 (13.1%) | 117 (68.4%) | 1 (0.7%) |
| Unknown | 15 (2.9%) | 33 (11.7%) | 18 (10.5%) | 9 (6.1%) |
| Grade (n) | | | | |
| G2 | 19 (3.7%) | 114 (40.3%) | 81 (47.4%) | 2 (1.4%) |
| G3 | 67 (12.9%) | 104 (36.7%) | 70 (40.9%) | 0 (0%) |
| G4 | 419 (80.6%) | 32 (11.3%) | 2 (1.2%) | 137 (92.6%) |
| Unknown | 15 (2.9%) | 33 (11.7%) | 18 (10.5%) | 9 (6.1%) |
| Age | | | | |
| Median (LQ-UQ) | 59 (51-68) | 38 (30-44) | 46 (35-54) | 55 (48-68) |
| Unknown (n) | 16 | 33 | 18 | 9 |
| Survival | | | | |
| Median (CI) | 14.0 (12.6-15.3) | 75.1 (62.1-94.5) | 115.8 (90.5-Inf) | 12.6 (11.3-14.9) |
| Unknown (n) | 14 | 32 | 18 | 12 |
| KPS | | | | |
| <70 | 85 (16.3%) | 8 (2.8%) | 5 (2.9%) | 21 (14.2%) |
| 70-80 | 196 (37.7%) | 41 (14.5%) | 18 (10.5%) | 60 (40.5%) |
| 90 | 29 (5.6%) | 60 (21.2%) | 32 (18.7%) | 2 (1.4%) |
| 100 | 51 (9.8%) | 44 (15.9%) | 30 (17.5%) | 14 (9.5%)‘ |
| Unknown | 159 (30.6%) | 129 (45.6%) | 86 (50.3%) | 51 (34.5%) |
| Molecular | | | | |
| MGMT promoter | | | | |
| Methylated | 170 (32.7%) | 242 (85.5%) | 169 (98.8%) | 32 (21.6%) |
| Unmethylated | 248 (47.7%) | 36 (12.7%) | 1 (0.6%) | 34 (23.0%) |
| Unknown | 102 (19.6%) | 5 (1.8%) | 1 (0.6%) | 82 (55.4%) |
| TERT promoter | | | | |
| Mutant | 67 (12.9%) | 8 (2.8%) | 86 (50.3%) | 1 (0.7%) |
| Wild-type | 19 (9.8%) | 146 (51.6%) | 2 (1.2%) | 0 (0%) |
| Unknown | 434 (83.5%) | 129 (45.6%) | 83 (48.5%) | 135 (99.3%) |
| TERT expression | | | | |
| Expressed | 178 (34.2%) | 14 (4.9%) | 153 (89.5%) | 6 (4.1%) |
| Not expressed | 51 (9.8%) | 242 (85.5%) | 16 (9.4%) | 7 (4.7%) |
| Unknown | 291 (56.0%) | 27 (9.5%) | 2 (1.2%) | 135 (91.2%) |

### Example 2: Identification of Novel Glioma-Associated Genomic Alterations

To establish the set of genomic alterations that drive gliomagenesis, point mutations and indels were called on the exomes of 513 LGG and 307 GBM using the Mutect, Indelocator, Varscan2, and RADIA algorithms. All mutations identified by at least two callers were considered. Significantly mutated genes (SMGs) were determined using MutSigCV. This led to the identification of 75 SMGs, 10 of which had been previously reported in GBM (Brennan et al., 2013), 12 of which had been reported in LGG (Cancer Genome Atlas Research Network et al., 2015), and 8 of which had been identified in both GBM and LGG studies.

45 SMGs had not been previously associated with glioma and ranged in mutation frequency from 0.5% to 2.6%. GISTIC2 was used to analyze the DNA copy number profiles of 1,084 samples, including 513 LGG and 571 GBM, and identified 162 significantly altered DNA copy number segments. PRADA and deFuse were employed to detect 1,144 gene fusion events in the RNA-seq profiles available for 154 GBM and 513 LGG samples, of which 37 in-frame fusions involved receptor tyrosine kinases.

Collectively, these analyses recovered all known glioma driving events, including in IDH1 (n=457), TP53 (n=328), ATRX (n=220), EGFR (n=314), PTEN (n=168), CIC (n=80), and FUBP1 (n=45). Notable newly predicted glioma drivers relative to the earlier TCGA analyses were genes associated with chromatin organization such as SETD2 (n=24), ARID2 (n=20), DNMT3A (n=11), and the KRAS/NRAS oncogenes (n=25 and n=5, respectively).

Overlapped copy number, mutation (n=793), and fusion transcript (n=649) profiles confirmed the convergence of genetic drivers of glioma into pathways, including the Ras-Raf-MEK-ERK, p53/apoptosis, PI3K/AKT/mTOR, chromatin modification, and cell cycle pathways. The Ras-Raf-MEK-ERK signaling cascade showed alterations in 106 of 119 members detected across 578 cases (73%), mostly occurring in IDH-wild-type samples (n=327 of 357, 92%). Conversely, a set of 36 genes involved in chromatin modification was targeted by genetic alterations in 423 tumors (54%, n=36 genes), most of which belonged to the IDH mutant-non-codel group (n=230, 87%).

In order to identify new somatically altered glioma genes, MutComFocal was used to nominate candidates altered by mutation, as well as copy number alteration. Prominent among these genes was NIPBL, a crucial adherin subunit that is essential for loading cohesins on chromatin (Peters and Nishiyama, 2012). The cohesin complex is responsible for the adhesion of sister chromatids following DNA replication and is essential to prevent premature chromatid separation and faithful chromosome segregation during mitosis (Peters and Nishiyama, 2012). Alterations in the cohesin pathway have been reported in 12% of acute myeloid leukemias (Kon et al., 2013). Mutations of the cohesin complex gene STAG2 had been previously reported in GBM (Brennan et al., 2013). Taken together, 16% of the LGG/GBM showed mutations and/or CNAs in multiple genes involved in the cohesin complex, thus nominating this process as a prominent pathway involved in gliomagenesis.

### Example 3: Telomere Length Is Positively Correlated with ATRX, but Not TERT Promoter Mutations

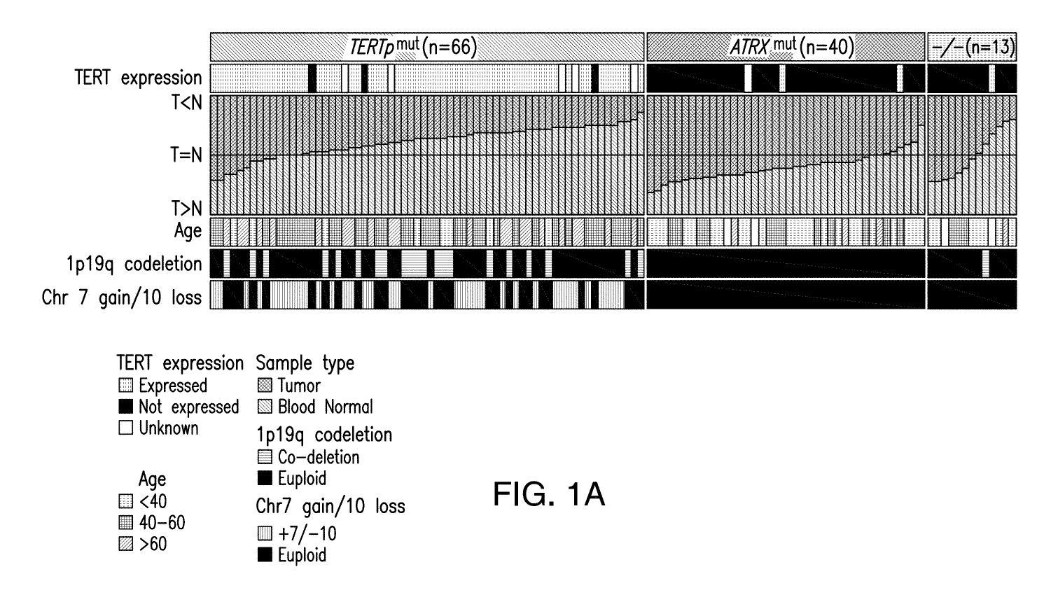

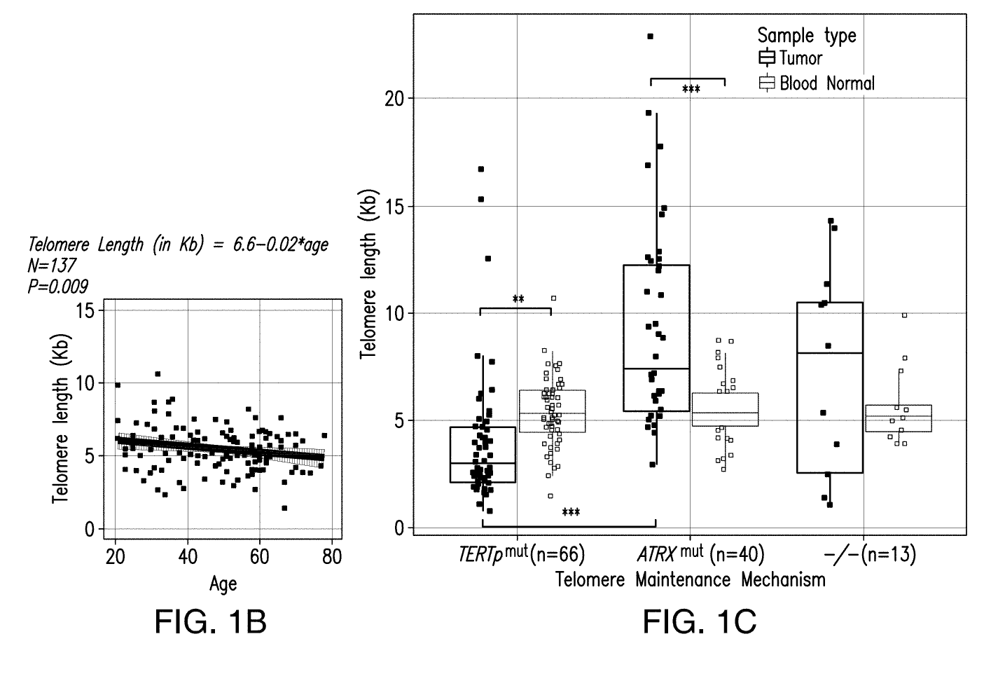

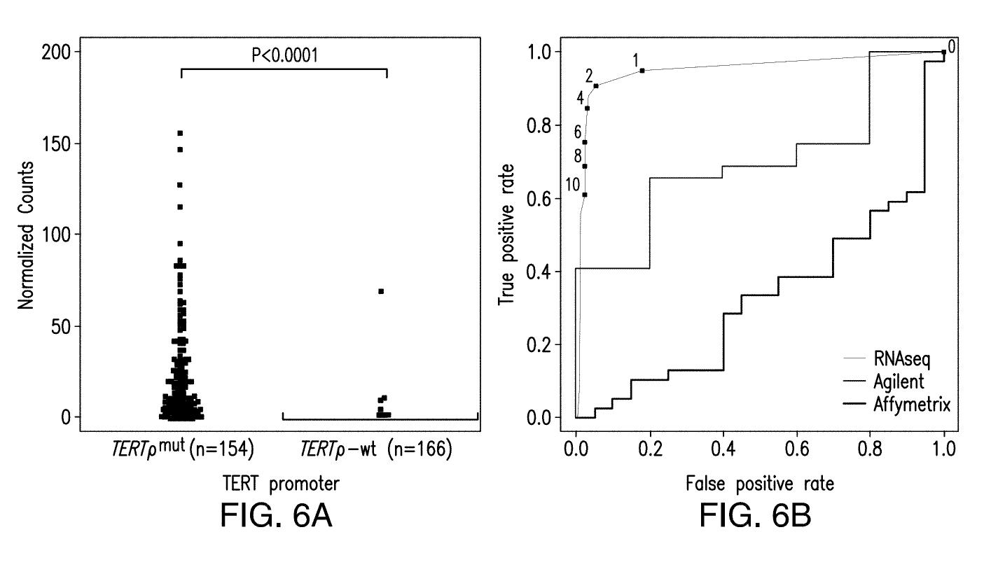

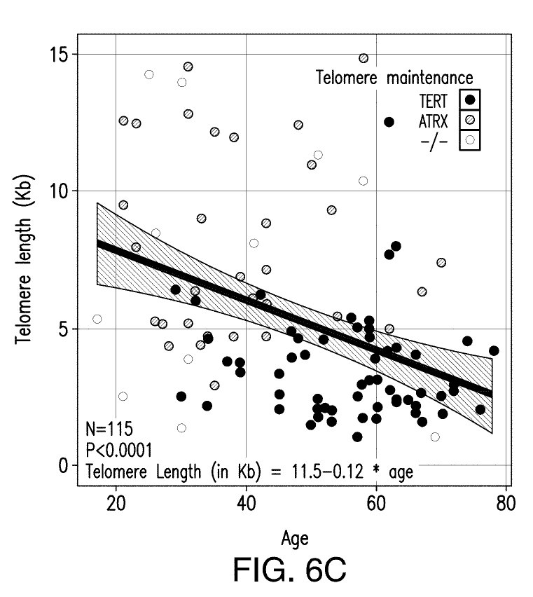

Mutations in the TERT promoter (TERTp) have been reported in 80% of GBM (Killela et al., 2013). TERTp mutation calls were used from targeted sequencing (n=287) were complemented with TERTp mutations inferred from whole-genome sequencing (WGS) data (n=42). TERTp mutations are nearly mutually exclusive with mutations in ATRX (Eckel-Passow et al., 2015), which was confirmed in in this Example. Overall, 85% of diffuse gliomas harbored mutations of TERTp (n=157, 48%) or ATRX (n=120, 37%). TERTp mutations activate TERT mRNA expression through the creation of a de novo E26 transformation-specific (ETS) transcription factor-binding site (Horn et al., 2013), and significant TERT upregulation was observed in TERTp mutant cases (p value <0.0001, FIG. 6A). TERT expression measured by RNA-seq was a highly sensitive (91%) and specific (95%) surrogate for the presence of TERTp mutation (FIG. 6B). TERTp status ws correlated with glioma driving alterations and nearly all IDH-wild-type cases with chromosome 7 gain and chromosome 10 loss harbored TERTp mutations or upregulated TERT expression (n=52/53 and n=134/147, respectively; FIG. 1A). Conversely, only 45% of IDH-wild-type samples lacking chromosome 7/chromosome 10 events showed TERTp mutations or elevated TERT expression (n=15/33 and n=43/82, respectively). Thus, TERTp mutations may precede the chr 7/chr 10 alterations that have been implicated in glioma initiation (Ozawa et al., 2014).

To correlate TERTp mutations to telomere length, whole-genome sequencing and low pass whole-genome sequencing data were used to estimate telomere length in 141 pairs of matched tumor and normal samples. As expected, an inverse correlation of telomere length with age at diagnosis was observed in matching blood normal samples (FIG. 1B) and tumor samples (FIG. 6C). Glioma samples harboring ATRX mutations showed significantly longer telomeres compared to TERTp mutant samples (t test p value <0.0001; FIG. 1C). Among TERTp mutation gliomas, there was no difference in telomere length between samples with and without additional IDH1/IDH2 mutations, despite a difference in age. ATRX forms a complex with DAXX and H3.3, and the genes encoding these proteins are frequently mutated in pediatric gliomas (Sturm et al., 2012). Mutations in DAXX and H3F3A were identified in only two samples in the WGS dataset. The ATRX-DAXX-H3.3 complex is associated with the alternative lengthening of telomeres (ALT) and the data herein confirmed the previously hypothesized fundamental differences between the telomere control exerted by telomerase and ALT (Sturm et al., 2014).

As demonstrated by the identification of TERTp mutations, somatic variants affecting regulatory regions may play a role in gliomagenesis. Using 67 matched whole-genome and RNA-seq expression pairs, mutations located within 2 kb upstream of transcription start sites and associated with a gene expression change were identified. Using strict filtering methods, 12 promoter regions with mutations in at least 6 samples were identified. Three of 12 regions related to a significant difference in the expression of the associated gene expression, suggesting possible functional consequences. Other than TERT (n=37), promoter mutations of the ubiquitin ligase TRIM28 (n=8) and the calcium channel gamma subunit CACNG6 (n=7) correlated with respectively upregulation and downregulation of these genes, respectively. TRIM28 has been reported to mediate the ubiquitin-dependent degradation of AMP-activated protein kinase (AMPK) leading to activation of mTOR signaling and hypersensitization to AMPK agonists, such as metformin (Pineda et al., 2015).

### Example 4: Unsupervised Clustering of Gliomas Identifies Six Methylation Groups and Four RNA Expression Groups Associated with IDH Status

To segregate the DNA methylation subtypes across the pan-glioma dataset, 932 glioma samples were profiled on the HumanMethylation450 platform (516 LGG and 129 GBM) and the HumanMethylation27 platform (287 GBM). In order to incorporate the maximum number of samples, datasets from both methylation platforms were mereged yielding a core set of 25,978 CpG probes. To reduce computational requirements to cluster this large dataset, sites that were methylated (mean β value ≥0.3) in non-tumor brain tissues were eliminated and 1,300 tumor-specific methylated probes (1,300/25,978, 5%) were selected to perform unsupervised k-means consensus clustering. This identified six distinct clusters, labeled LGm1-6 (FIG. 2A).

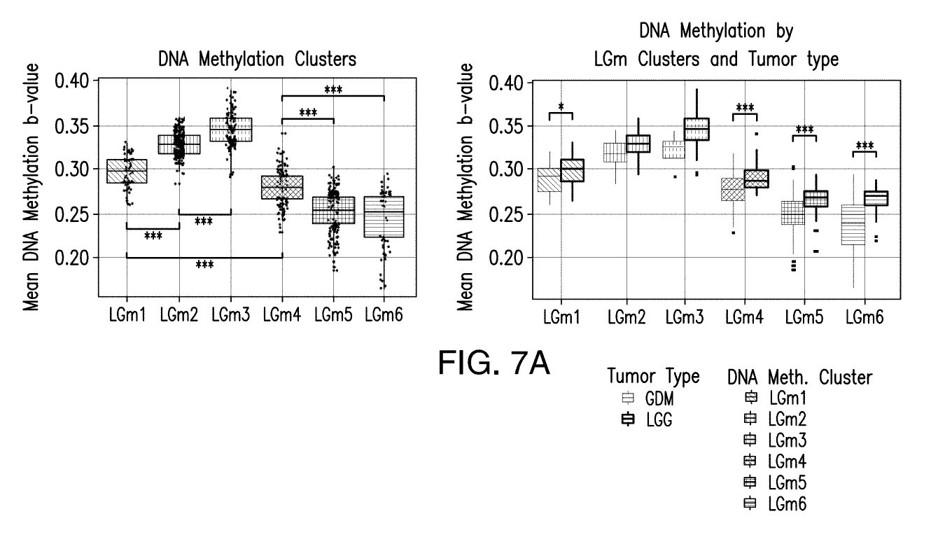

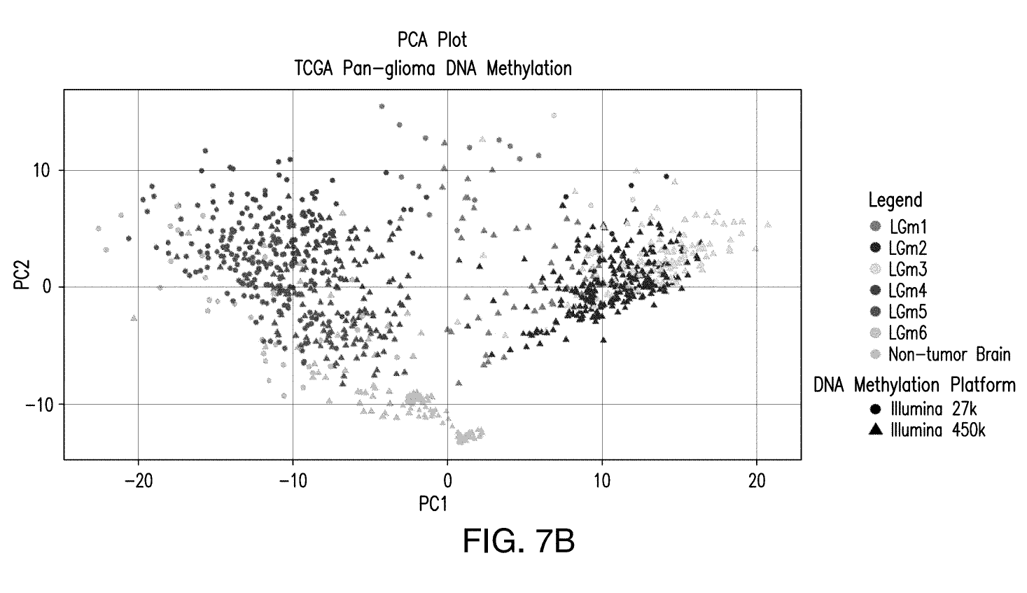

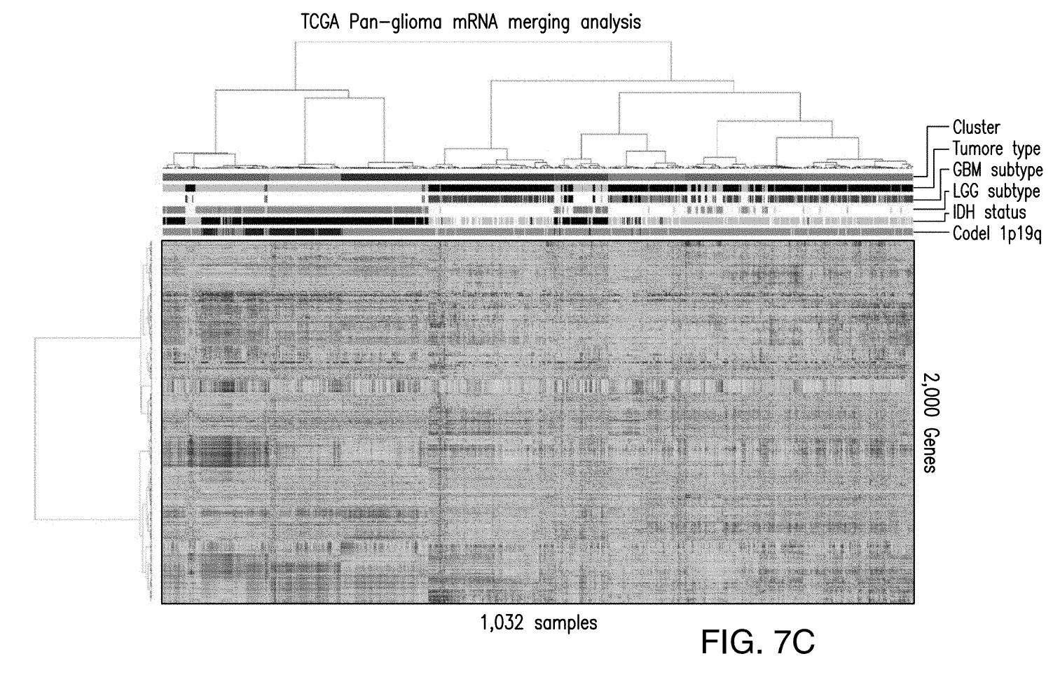

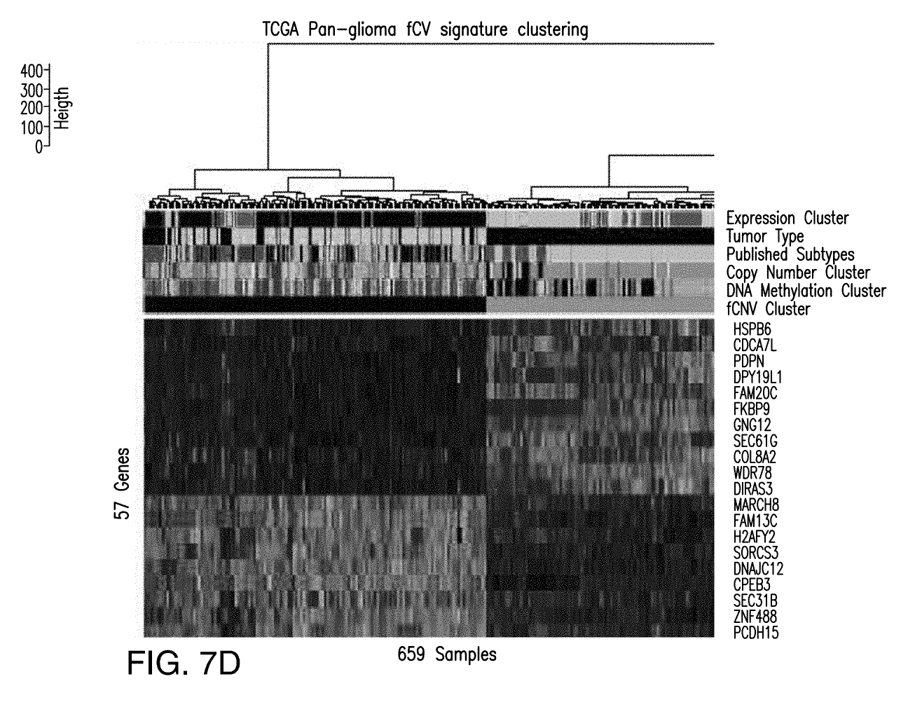

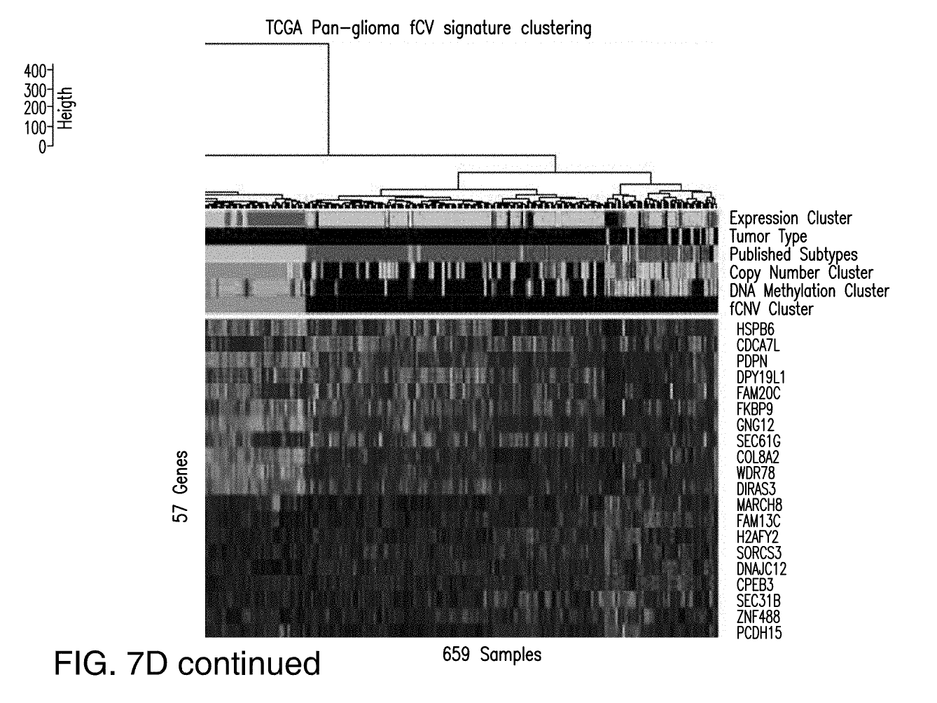

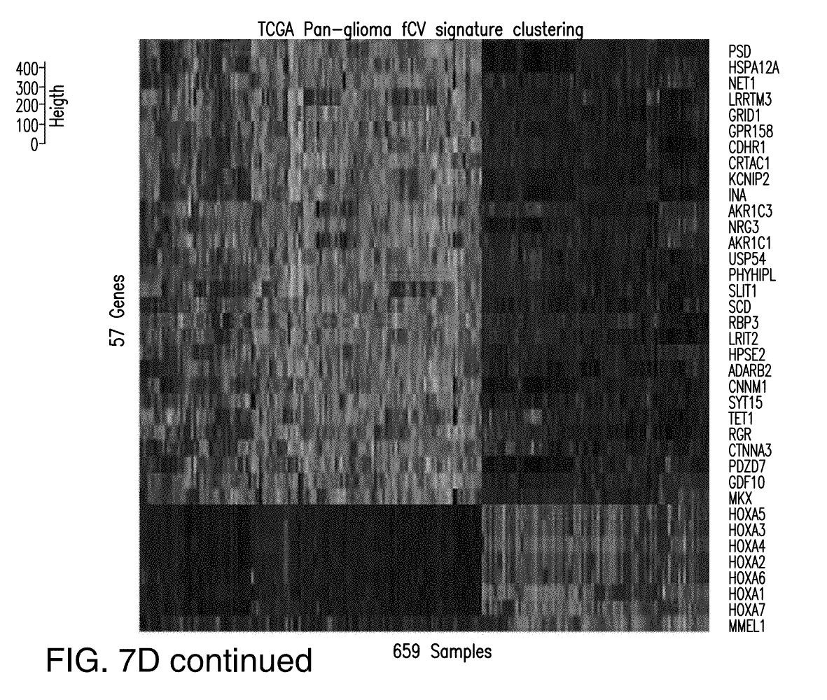

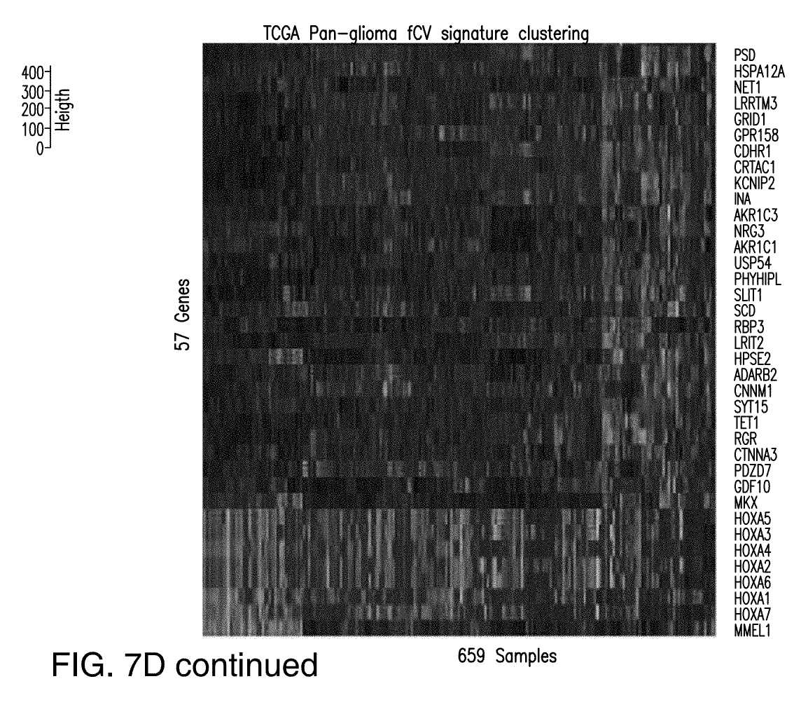

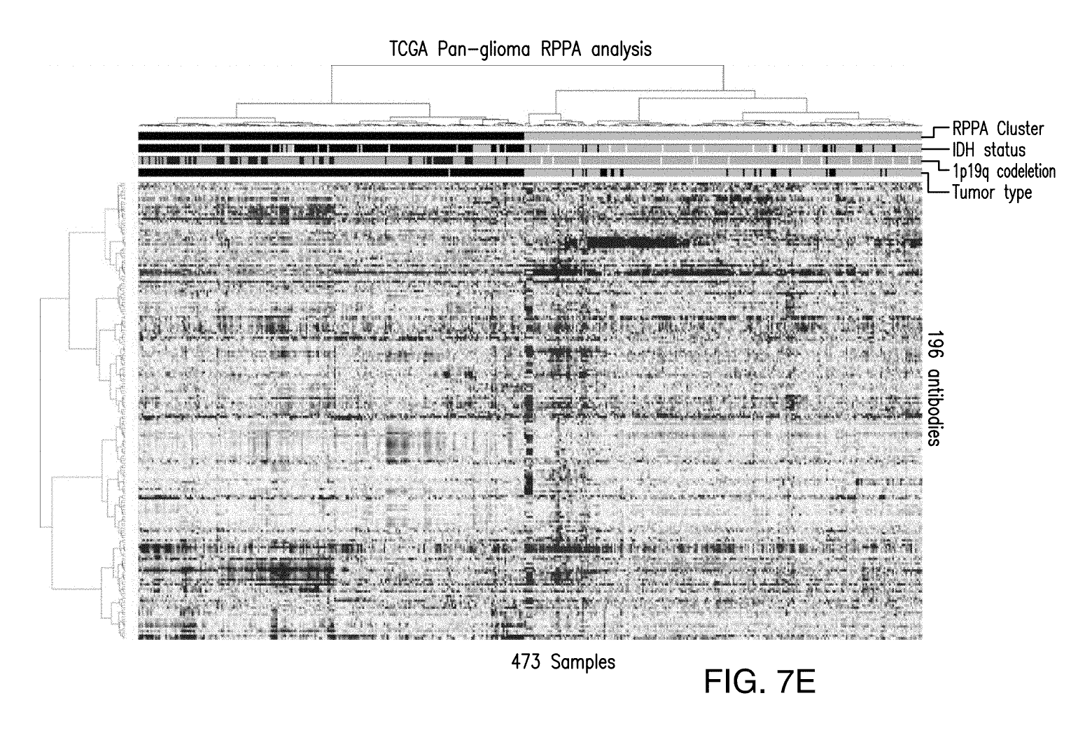

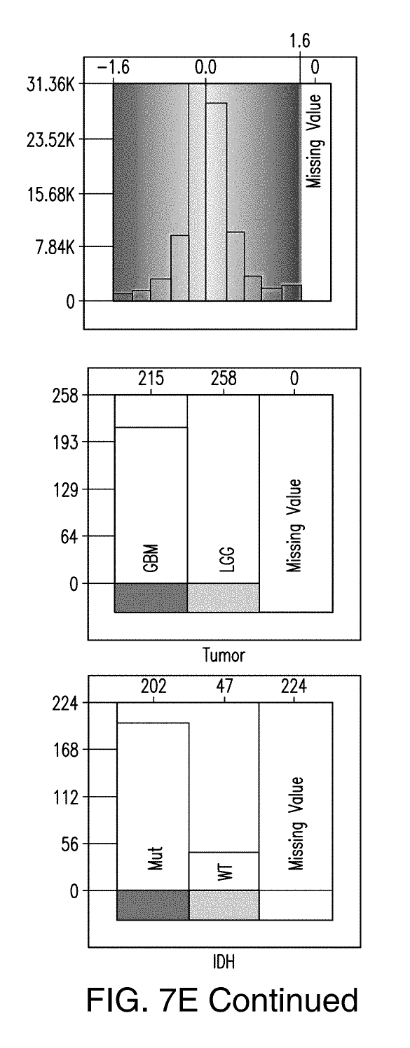

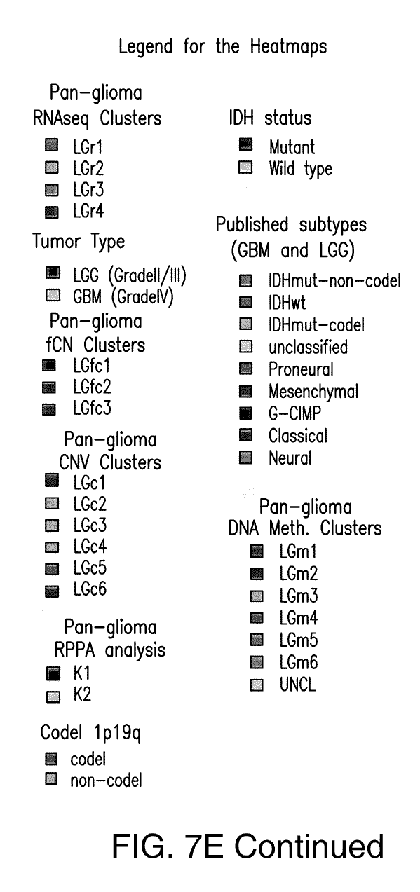

Next, pan-glioma expression subtypes were determined through unsupervised clustering analysis of 667 RNA-seq profiles (513 LGG and 154 GBM), which resulted in four main clusters labeled LGr1-4 (FIG. 2B). An additional 378 GBM samples with Affymetrix HT-HG-U133A profiles (but lacking RNA-seq data) were classified into the four clusters using a k-nearest neighbor classification procedure. IDH mutation status was the primary driver of methylome and transcriptome clustering and separated the cohort into two macro-groups. The LGm1/LGm2/LGm3 DNA methylation macro-group carried IDH1 or IDH2 mutations (449 of 450, 99%) and was enriched for LGG (421/454, 93%) while LGm4/LGm5/LGm6 were IDH-wild-type (429/430, 99%) and enriched for GBM (383/478, 80%). LGm1-3 showed genome-wide hypermethylation compared to LGm4-6 clusters (FIG. 7A), documenting the association between IDH mutation and increased DNA methylation (Noushmehr et al., 2010, Turcan et al., 2012). Principal component analysis using 19,520 probes yielded similar results, thus emphasizing that this probe selection method did not introduce unwanted bias (FIG. 7B). The gene expression clusters LGr1-3 harbored IDH1 or IDH2 mutations (438 of 533, 82%) and were enriched for LGG (436/563, 77%), while the LGr4 was exclusively IDH-wild-type (376 of 387, 97%) and enriched for GBM (399/476, 84%).

The analysis was extended using Tumor Map to perform integrated co-clustering analysis of the combined gene expression (n=1,196) and DNA methylation (n=867) profiles. Tumor Map assigns samples to a hexagon in a grid so that nearby samples are likely to have similar genomic profiles and allows visualizing complex relationships between heterogeneous genomic data samples and their clinical or phenotypical associations. Thus, clusters in the map indicate groups of samples with high similarity of integrated gene expression and DNA methylation profiles (FIG. 2C). The map confirms clustering by IDH status and additionally shows islands of samples that share previously reported GBM cluster memberships (Noushmehr et al., 2010, Verhaak et al., 2010). To assess clustering sensitivity to pre-processing, complementary methods were tried and similar results were obtained (FIG. 7C).

To identify genes whose copy number changes are associated with concordant changes in gene expression, expression and copy number profiles were combined from 659 samples to define a signature of 57 genes with strong functional copy number (fCN) change. The fCN signature clustered gliomas into three macro-clusters, LGfc1-3, strongly associated with IDH and 1p/19q status (FIG. 7D). The fCN analysis revealed the functional activation of a cluster of HOXA genes in the IDH-wild-type LGfc2 cluster, which were previously associated with glioma stem cell maintenance (Kurscheid et al., 2015).

Reverse phase protein array profiles, consisting of 196 antibodies on 473 samples were clustered. Two macro clusters were observed, and in contrast to the transcriptome/methylome/fCNV clustering, the primary discriminator was based on glioma grade (LGG versus GBM) rather than IDH status (FIG. 7E). Compared to the LGG-like cluster, the GBM-like cluster had elevated expression of IGFBP2, fibronectin, PAIL HSP70, EGFR, phosphoEGFR, phosphoAKT, Cyclin B1, Caveolin, Collagen VI, Annexin1, and ASNS, whereas the LGG class showed increased activity of PKC (alpha, beta, and delta), PTEN, BRAF, and phosphoP70S6K.

These results confirmed IDH status as the major determinant of the molecular footprints of diffuse glioma. To further elucidate the subtypes of diffuse glioma, unsupervised clustering within each of the two IDH-driven macroclusters was performed. 1,308 tumor-specific CpG probes defined among the IDH mutation cohort (n=450) were used and three IDH mutant-specific DNA methylation clusters were identified (FIG. 8A). Using 914 tumor-specific CpG probes in the IDH-wild-type cohort (n=430), three IDH-wild-type-specific clusters were uncovered (FIG. 9A). The sets of CpG probes used to cluster each of the two IDH-driven datasets overlapped significantly with the 1,300 probes that defined the pan-glioma DNA methylation clustering (1162/1,300, 89% and 853/1,300, 66%, for IDH mutant and IDH-wild-type, respectively). The clusters identified by separating IDH mutant and IDH-wild-type gliomas showed strong overall concordance with pan-glioma DNA methylation subtypes. Similarly, unsupervised clustering of 426 IDH mutant RNA-seq profiles resulted in three subtypes (FIG. 8A), and analysis of the 234 IDH-wild-type samples led to four mixed LGG/GBM clusters that showed enrichment for previously identified GBM expression subtypes (FIG. 9C) (Verhaak et al., 2010).

### Example 5: An Epigenetic Signature Associated with Activation of Cell Cycle Genes Segregates a Subgroup of IDH Mutant LGG and GBM with Unfavorable Clinical Outcome

The three epigenetic subtypes defined by clustering IDH mutant glioma separated samples harboring the 1p/19q co-deletion into a single cluster and non-codel glioma into two clusters (FIG. 8A). Conversely, non-codel glioma grouped nearly exclusively into a single expression cluster, and codels were split in two separated expression clusters (FIG. 8A). A distinct subgroup of samples within the IDH mutant-non-codel DNA methylation clusters manifested relatively reduced DNA methylation (FIG. 8B). The unsupervised clustering of IDH mutant glioma was unable to segregate the lower methylated non-codel subgroup as the 1,308 probes selected for unsupervised clustering included only 19 of the 131 differentially methylated probes characteristic for this subgroup (FDR<10-15, difference in mean methylation beta value >0.27). The low-methylation subgroup consisted of both G-CIMP GBM (13/25) and LGGs (12/25) and was confirmed using a non-TCGA dataset (FIG. 8C).

The tumors with higher methylation in the split cluster were very similar to those grouped in the second non-codel cluster, and a supervised comparison identified only 12 probes as differentially DNA methylated (FIG. 3A and FIG. 3B). Thus, IDH mutant glioma is composed of three coherent subgroups: (1) the Codel group, consisting of IDH mutant-codel LGGs; (2) the G-CIMP-low group, including IDH mutant-non-codel glioma (LGG and GBM) manifesting relatively low genome-wide DNA methylation; and (3) the G-CIMP-high group, including IDH mutant-non-codel glioma (LGG and GBM) with higher global levels of DNA methylation. The newly identified G-CIMP-low group of glioma was associated with significantly worse survival as compared to the G-CIMP-high and Codel groups (FIG. 8D). The clinical outcome of the tumors classified as G-CIMP-high was as favorable as that of Codel tumors, the subgroup generally thought to have the best prognosis among glioma patients (FIG. 3C and FIG. 8D). The frequencies of glioma driver gene alterations between the three types of IDH mutant glioma were compared and showed that 15 of 18 G-CIMP-low cases carried abnormalities in cell cycle pathway genes such as CDK4 and CDKN2A, relative to 36/241 and 2/172 for G-CIMP-high and Codels, respectively (FIG. 3D). Supervised analysis between gene expression of G-CIMP-low and G-CIMP-high resulted in 943 differentially expressed genes. The 943 deregulated genes were mapped to 767 nearest CpG probes (max distance 1 kb) and the majority of the CpG probes (486/767, 63%) were found to show a significant methylation difference (FDR<0.05, difference in mean methylation beta value >0.01) between G-CIMP-low and G-CIMP-high, suggesting a mechanistic relation between loss of methylation and increased transcript levels.

Recent analysis of epigenetic profiles derived from colon cancers showed that transcription factors may bind to regions of demethylated DNA (Berman et al., 2012). Therefore, it was determined whether transcription factors may be recruited to the DNA regions differentially methylated between G-CIMP-low samples and G-CIMP-high samples from the same methylation cluster, using 450K methylation profiles (n=39). Globally, 643 differentially methylated probes between 27 G-CIMP-low and 12 G-CIMP-high samples (absolute diff-mean difference ≥0.25, FDR≤5%) were detected. Most of these probes (69%) were located outside of any known CpG island but positioned within intergenic regions known as open seas (FIG. 3E). This represents a 2.5-fold open sea enrichment compared to the expected genome-wide distribution of 450K CpG probes (chi-square p value <2.2×10-16). A 3.4-fold depletion within CpG islands (chi-square p value <2.2×10-16) was also observed.

Using this set of intergenic CpG probes, it was determined whether a DNA motif signature associated with distal regulatory elements. Such a pattern would point to candidate transcription factors involved in tumorigenesis of the G-CIMP-low group. A de novo motif scan and known motif scan identified a distinct motif signature TGTT (geometric test p value=10-11, fold enrichment=1.8), known to be associated with the OLIG2 and SOX transcription factor families (FIG. 3E) (Lodato et al., 2013). This observation was corroborated by the higher expression levels of SOX2, as well as 17 out of 20 other known SOX family members in G-CIMP-low compared to G-CIMP-high (fold difference >2). The primary function of SOX2 in the nervous system is to promote self-renewal of neural stem cells and, within brain tumors, the glioma stem cell state (Graham et al., 2003). Interestingly, SOX2 and OLIG2 have been described as neurodevelopmental transcription factors being essential for GBM propagation (Suva et al., 2014). Supervised gene expression pathway analysis of the genes activated in the G-CIMP-low group as opposed to G-CIMP-high group revealed activation of genes involved in cell cycle and cell division consistent with the role of SOX in promoting cell proliferation (FIG. 8E). The enrichment in cell cycle gene expression provides additional support to the conclusion that development of the G-CIMP-low subtype is associated with activation of cell cycle progression and may be mediated by a loss of CpG methylation and binding of SOX factors to candidate genomic enhancer elements.

To validate the G-CIMP-low, G-CIMP-high, and Codel IDH mutant subtypes, a validation cohort was compiled from published studies, including 324 adult and pediatric gliomas (Lambert et al., 2013, Mur et al., 2013, Sturm et al., 2012, Turcan et al., 2012). CpG probe methylation signatures were used to classify the validation set. Among them, 103 were identified as IDH mutant on the basis of their genome-wide DNA methylation profile. Samples in the validation set were classified using the probes that defined the IDH mutant-specific DNA methylation cluster analysis integrated in a supervised random forest method. The analysis recapitulated the clusters generated from the TCGA collection (FIG. 8C). In order to determine epigenetically regulated (EReg) genes that may be characteristic of the biology of the IDH mutant diffuse glioma subtypes, 450 k methylation DNA methylation profiles and gene expression levels were compared between 636 IDH mutant and IDH-wild-type gliomas and 110 non-tumor samples from 11 different tissue types. From the list of epigenetically regulated genes, 263 genes that were grouped into EReg gene signatures were extracted, which showed differential signals among the three IDH mutant subtypes (FIG. 3F). These trends were confirmed in the validation set (FIG. 3G).

The possibility that the G-CIMP-high group is a predecessor to the G-CIMP-low group was investigated by comparing the DNA methylation profiles from ten IDH mutant-non-codel LGG and GBM primary-recurrent cases with the TCGA cohort. The DNA methylation status of probes identified as differentially methylated (n=90) between G-CIMP-low and G-CIMP-high (FDR<10-13, difference in mean methylation beta-value >0.3 and <−0.4) was evaluated. Four out of ten IDHmut-non-codel cases showed a demethylation pattern after disease recurrence, while partial demethylation was demonstrated in the remaining six recurrences, supporting the notion of a progression from G-CIMP-high to G-CIMP-low phenotype (FIG. 3H).

### Example 6: An IDH-Wild-Type Subgroup of Histologically Defined Diffuse Glioma Is Associated with Favorable Survival and Shares Epigenomic and Genomic Features with Pilocytic Astrocytoma

IDH-wild-type gliomas segregated into three DNA methylation clusters (FIG. 9A). The first is enriched with tumors belonging to the classical gene expression signature and was labeled Classic-like, whereas the second group, enriched with mesenchymal subtype tumors, was labeled Mesenchymal-like ((Verhaak et al., 2010). The third cluster contained a larger fraction of LGG in comparison to the other IDH-wild-type clusters. The IDH-wild-type LGGs but not the IDH-wild-type GBM in this cluster displayed markedly longer survival (log-rank p value=3.6×10-5; FIG. 4A) and occurred in younger patients (mean 37.6 years versus 50.8 years, t test p value=0.002). Supervised analysis of differential methylation between LGG and GBM in the third DNA methylation cluster did not reveal any significant probes despite significant differences in stromal content (p value <0.005; FIG. 9D), suggesting that this group cannot be further separated using CpG methylation markers.

Next, the methylation-based classification of IDH-wild-type glioma was validated in an independent cohort of 221 predicted IDH-wild-type glioma samples, including 61 grade I pilocytic astrocytomas (PAs). A supervised random forest model built with the probes that defined the IDH-wild-type clusters was used. Samples classified as Mesenchymal-like showed enrichment for the Sturm et al. (2012)) Mesenchymal subtype (29/88), and gliomas predicted as Classic-like were all RTK II “Classic” (22/22), per the Sturm et al. (2012)) classification (FIG. 4B and FIG. 9B). PA tumors were unanimously classified as the third, LGG-enriched group (FIG. 9B). Based on the molecular similarity with PA, the LGGs in the third methylation cluster of IDH-wild-type tumors were labeled as PA-like. The GBMs in this group were best described as LGm6-GBM for their original pan-glioma methylation cluster assignment and tumor grade.

Pilocytic astrocytomas are characterized by frequent alterations in the MAPK pathway, such as FGFR1 mutations, KIAA1549-BRAF, and NTRK2 fusions (Jones et al., 2013). The frequency of mutations, fusions, and amplifications in eight PA-associated genes (BRAF, NF1, NTRK1, NTRK2, FGFR1, and FGFR2) rated from 11% (n=12/113) of Classic-like, 13% (n=21/158) of Mesenchymal-like IDH-wild-type tumors to 32% (n=7/22) of LGm6-GBM and 52% (n=13/25) of PA-like LGG (Fisher's exact test [FET] p value <0.0001; FIG. 4C). Conversely, only 2 of 25 (8%) PA-like LGG tumors showed TERT expression, compared to 5 of 12 LGm6-GBM (43%), 60 of 65 Classic-like (92%), and 82 of 98 Mesenchymal-like (84%, FET p value <0.0001). The PA-like group was characterized by relatively low frequency of typical GBM alterations in genes such as EGFR, CDKN2A/B, and PTEN and displayed euploid DNA copy number profiles (FIG. 9E). To ascertain that the histologies of the PA-like subgroup had been appropriately classified, an independent re-review was conducted. This analysis confirmed the presence of the histologic features of diffuse glioma (grade II or grade III) in 23 of the 26 cases in the cluster. The remaining three cases were re-named as PA (grade I). An independent review of the magnetic resonance diagnostic images from 13 cases showed a similar pattern, with the majority of tumors showing behavior consistent with grade II or grade III glioma. Taken together, the epigenetic analysis of the IDH-wild-type group of adult glioma revealed the existence of a novel subgroup sharing genetic and DNA methylation features with pediatric PA and favorable clinical outcome compared to diffuse IDH-wild-type glioma. This group may include but extends beyond BRAF-mutated grade II oligodendroglioma that were previously recognized as a unique clinical entity (Chi et al., 2013).

Through comparison of the methylation profiles of 636 glioma and 110 non-neoplastic normal samples from different tissue types, EReg signatures were defined consisting of 27 genes that showed differential signals among IDH-wild-type subtypes in the TCGA (FIG. 4D) and the validation set (FIG. 4E). EReg4 comprised a group of 15 genes hypermethylated and downregulated in particularly Classic-like. EReg5 was defined as a group of 12 genes associated with hypomethylation in LGm6/PA-like compared to all other LGm clusters. These ERegs aided in characterizing the biological importance of IDH-wild-type subtypes and were subsequently used to evaluate the prognostic importance of the IDH-wild-type clusters.

### Example 7: The Epigenetic Classification of Glioma Provides Prognostic Value Independent of Age and Grade

In order to assess whether the DNA methylation-based subtypes identified carry prognostically relevant information independent of known overall survival predictors, a series of survival regression models were conducted. To find the optimal model for survival prediction, covariates were studied individually and in combination with other covariates. Age at diagnosis, histology, IDH/codel subtype, TERT expression, and epigenetic subtype all contribute to survival in single-predictor analysis (log-rank p value <0.05). As expected, age was a highly significant predictor (p<0.0001, C-Index 0.78) and was included in all subsequent multi-predictor models. Histology and grade are highly correlated. Histology provided only marginal improvement to a model that includes grade (likelihood ratio test [LRT] p value=0.08) and was therefore not included in further analyses. Conversely, grade markedly impacted a histology-based predictor model (LRT p value=0.0005) and was retained in the subsequent models. In contrast to previous reports (Eckel-Passow et al., 2015), a statistically significant and independent survival association with TERT expression (LRT p value=0.82) or TERTp mutations after accounting for age and grade (LRT p value=0.85, data not shown) was not observed. Thus, the optimal survival prediction model includes age, grade, and epigenetic subtype (LRT p value <0.0001, C-Index 0.836; Table 2).

In Table 2, survival regression analysis indicates that an optimal model of prognosis includes age, grade, and methylation subtype. These predictors were statistically significant in both the discovery dataset and an external validation dataset. Significance codes: 0 “***”; 0.001 “**”; 0.05 “*”; 0.1 “.”

TABLE 2
DNA Methylation Subtypes Are Prognostically Relevant in Multivariable
Analysis and in External Validation Data

| | | Discovery (n = 809) | | |
|---|---|---|---|---|
| | | C-Index: 0.835 ± 0.019 | | |
| | | | HR (95% | |
| Predictor | Levels | n | CI) | Signif. |
| Age at diagnosis | per year | 809 | 1.05 (1.03-1.06) | *** |
| WHO Grade | II | 214 | 1.0 (ref) | |
| | III | 241 | 1.96 (1.15-3.33) | * |
| | IV | 354 | 2.38 (1.3-4.34) | * |
| Subgroup | IDHmut-codel | 156 | 1.0 (ref) | |
| | G-CIMP-low | 22 | 5.6 (2.49-12.62) | *** |
| | G-CIMP-high | 219 | 1.92 (1.05-3.51) | * |
| | classic-like | 143 | 5.4 (2.79-10.44) | *** |
| | mesenchymal- | 204 | 8.71 (4.59-16.53) | *** |
| | like I | | | |
| | LGm6-GBM | 39 | 5.79 (2.78-12.1) | *** |
| | PA-like | 26 | 2.02 (0.71-5.71) | |
| | | Validation (n = 183) | | |
| | | C-Index: 0.745 ± 0.032 | | |
| | | | HR (95% | |
| Predictor | Levels | n | CI) | Signif. |
| Age at diagnosis | per year | 183 | 1.02 (1-1.04) | * |
| WHO Grade | II | 41 | 1.0 (ref) | |
| | III | 51 | 1.24 (0.55-2.76) | |
| | IV | 91 | 2.6 (1.08-6.3) | * |
| Subgroup | IDHmut-codel | 57 | 1.0 (ref) | |
| | G-CIMP-low | 2 | 0 (0-Inf) | |
| | G-CIMP-high | 15 | 1.25 (0.43-3.66) | |
| | classic-like | 22 | 4.55 (1.8-11.49) | * |
| | mesenchymal- | 61 | 5.55 (2.52-12.21) | *** |
| | like I | | | |
| | LGm6-GBM | 22 | 6.8 (2.58-17.91) | ** |
| | PA-like | 4 | 3.64 (0.79-16.78) | |

To confirm that the epigenetic subtypes provide independent prognostic information, the survival model was tested on the validation dataset. Epigenetic subtypes in these samples were determined as described above. The distinction between LGm6-GBM and PA-like gliomas was made on the basis of tumor grade and not by DNA methylation signature. Using a subset of 183 samples in the validation cohort with known survival, age, and grade, epigenetic subtypes were found to be significant independent predictors of survival in the multivariate analysis (LRT p value <0.0001, C-Index 0.746, Table 2). This generalization of the model supports the epigenetic subtypes as a means to improve the prognostication of glioma.

### Example 8: Activation of Cell Cycle/Proliferation and Invasion/Microenvironmental Changes Marks Progression of LGG to GBM

In spite of morphological differences between LGG and GBM, such as high cell density and microvascular proliferation, clustering of gene expression profiles frequently grouped LGG and GBM together within the same subtype. Gene Set Enrichment Analysis of the genes activated in G-CIMP GBM as opposed to the IDH mutant-non-codel within LGr3 (FIG. 2B) revealed four major groups, including cell cycle and hyperproliferation, DNA metabolic processes, response to stress, and angiogenesis (FIG. 10A). These biological functions are consistent with the criteria based on mitotic index used by pathologists to discriminate lower and high-grade glioma and the significance of activated microglia for tumor aggressiveness (Roggendorf et al., 1996). Conversely, compared with the G-CIMP GBM, IDH mutant-non-codel LGG in LGr3 were characterized by enrichment of genes associated with neuro-glial functions such as ion transport and synaptic transmission, possibly suggesting a more differentiated nature. The comparison of co-clustered GBM and LGG in LGr3 by the PARADIGM algorithm that integrates DNA copy number and gene expression to infer pathway activity confirmed that GBMs express genes associated with cell cycle, proliferation, and aggressive phenotype through activation of a number of cell cycle, cell replication, and NOTCH signaling pathways whereas LGGs exhibit an enrichment of neuronal-differentiation-specific categories, including synaptic pathways (FIG. 10C).

The analysis of the genes activated in GBM versus the LGG component of LGr4, which grouped IDH-wild-type tumors, identified an inflammation and immunologic response signature characterized by the activation of several chemokines (CCL18, CXCL13, CXCL2, and CXCL3) and interleukins (IL8 and CXCR2) enriching sets involved in inflammatory and immune response, negative regulation of apoptosis, cell cycle and proliferation, and the IKB/NFKB kinase cascade Map (FIG. 10B). These characteristics suggest differences in the relative amount of microglia. The ESTIMATE method was used to estimate the relative presence of stromal cells, which revealed significantly lower (p value 10-6) stromal scores of LGG IDH-wild-type versus GBM IDH-wild-type (FIG. 10F) (Yoshihara et al., 2013). Resembling the functional enrichment for LGG within LGr3, functional enrichment of LGG IDH-wild-type in comparison to GBM within LGr4 showed activation in LGG of special glial-neuronal functions involved in ion transport, synaptic transmission, and nervous system development.

Transcription factors that may exert control over prominent gene expression programs, known as master regulators, were identified. Master regulator analysis comparing the IDH-wild-type group to the IDH mutant group revealed transcription factors that were upregulated in IDH-wild-type gliomas and showed an increase in expression of target genes, including NKX2-5, FOSL1, ETV4, ETV7, RUNX1, CEBPD, NFE2L3, ELF4, RUNX3, NR2F2, PAX8, and IRF1. No transcription factors (TFs) were found to be upregulated in IDH mutant gliomas relative to IDH-wild-type gliomas (at a log fold change >1).

### Example 9: Classification of New Glioma Samples Based on DNA Methylation Glioma Subtypes

New glioma samples can be classified into one of the glioma subtypes identified herein using a CpG probe methylation signature. First, all glioma samples should be divided by their known IDH status, separated into either IDHmutant and IDH-wildtype.

IDH-mutant is defined as those samples harboring any type of IDH1 or IDH2 mutation, such as those as described recently (TCGA_Network, 2015). IDH-wildtype refers to those samples with an intact IDH1 or IDH2. Samples can then be further classified accordingly.

IDH-mutant samples may be classified into one of the 3 glioma subtypes using a two-step Random Forest method. First, the sample may be analyzed using the 1,308 tumor specific CpG probes that define the IDHmut specific clusters (FIG. 8A). Next, the sample may be analyzed using the 163 CpG probes that define each TCGA IDH-mutant glioma subtype (FIG. 8C).

If the sample is identified as IDHmut-K1 or IDHmut-K2 using the 1,308 tumor specific CpG probes for IDH-mutant and as G-CIMP-low using the 163 CpG probes defined by a supervised analysis across IDH-mutant subgroups, the sample is classified as G-CIMP-low.

If the sample is identified as IDHmut-K1 or IDHmut-K2 using the 1,308 tumor specific CpG probes for IDH-mutant and as G-CIMP-high using the 163 CpG probes defined by a supervised analysis across IDH-mutant subgroups, the sample is classified as G-CIMP-high.

If the sample is identified as IDHmut-K3 using the 1,308 tumor specific CpG probes for IDH-mutant, the sample is classified as Codel.

IDH-wildtype samples can be classified using a single Random Forest machine-learning model applied with a signature defined by the 914 tumor specific CpG probes for IDH-wildtype (FIG. 9A and FIG. 9B). Samples falling into IDHwt-K3 (aka LGm6) may be subdivided based on grade as either LGm6-GBM and PA-like (LGG).

The following publications are referred to at various places in this specification and are incorporated by reference herein:
Benjamini, Y., and Hochberg, Y. (1995). Controlling the False Discovery Rate—a Practical and Powerful Approach to Multiple Testing. J Roy Stat Soc B Met 57, 289-300.
Berman, B. P., Weisenberger, D. J., Aman, J. F., Hinoue, T., Ramj an, Z., Liu, Y., Noushmehr, H., Lange, C. P., van Dijk, C. M., Tollenaar, R. A. et al. (2012)
Regions of focal DNA hypermethylation and long-range hypomethylation in colorectal cancer coincide with nuclear lamina-associated domains. Nat. Genet.; 44: 40-46
Bindea, G., Mlecnik, B., Hackl, H., Charoentong, P., Tosolini, M., Kirilovsky, A., Fridman, W. H., Pages, F., Trajanoski, Z., and Galon, J. (2009). ClueGO: a Cytoscape plug-in to decipher functionally grouped gene ontology and pathway annotation networks. Bioinformatics 25, 1091-1093.
Borah, S., Xi, L., Zaug, A. J., Powell, N. M., Dancik, G. M., Cohen, S. B., Costello, J. C., Theodorescu, D., and Cech, T. R. (2105) Cancer. TERT promoter mutations and telomerase reactivation in urothelial cancer. Science; 347: 1006-1010
Brennan, C. W., Verhaak, R. G., McKenna, A., Campos, B., Noushmehr, H., Salama, S. R., Zheng, S., Chakravarty, D., Sanborn, J. Z., Berman, S. H., et al. (2013). The somatic genomic landscape of glioblastoma. Cell 155, 462-477.
Cancer Genome Atlas Research, N. (2008). Comprehensive genomic characterization defines human glioblastoma genes and core pathways. Nature 455, 1061-1068.
Cancer Genome Atlas Research, N. (2013). Comprehensive molecular characterization of clear cell renal cell carcinoma. Nature 499, 43-49.
Cancer Genome Atlas Research, N. (2014a). Comprehensive molecular characterization of gastric adenocarcinoma. Nature 513, 202-209.
Cancer Genome Atlas Research, N. (2014b). Comprehensive molecular characterization of urothelial bladder carcinoma. Nature 507, 315-322.
Cancer Genome Atlas Research, N. (2015). Comprehensive, Integrative Genomic Analysis of Diffuse Lower-Grade Gliomas. N Engl J Med.; 372: 2481-2498
Carter, S. L., Cibulskis, K., Helman, E., McKenna, A., Shen, H., Zack, T., Laird, P. W., Onofrio, R. C., Winckler, W., Weir, B. A., et al. (2012). Absolute quantification of somatic DNA alterations in human cancer. Nat Biotechnol 30, 413-421.
Chen, Y. J., Campbell, H. G., Wiles, A. K., Eccles, M. R., Reddel, R. R., Braithwaite, A. W., and Royds, J. A. (2008). PAX8 regulates telomerase reverse transcriptase and telomerase RNA component in glioma. Cancer research 68, 5724-5732.
Chi, A. S., Batchelor, T. T., Yang, D., Dias-Santagata, D., Borger, D. R., Ellisen, L. W., Iafrate, A. J., and Louis, D. N. (2013) BRAF V600E mutation identifies a subset of low-grade diffusely infiltrating gliomas in adults. J. Clin. Oncol.; 31: e233-e236
Cibulskis, K., Lawrence, M. S., Carter, S. L., Sivachenko, A., Jaffe, D., Sougnez, C., Gabriel, S., Meyerson, M., Lander, E. S., and Getz, G. (2013). Sensitive detection of somatic point mutations in impure and heterogeneous cancer samples. Nature biotechnology 31, 213-219.
Colaprico, A., Silva, T. C., Olsen, C., Garofano, L., Cava, C., Garolini, D., Sabedot, T., Malta, T., Pagnotta, S. M., Castiglioni, I., et al. (2015). TCGAbiolinks: An R/Bioconductor package for integrative analysis of TCGA data. doi: 10.1093/nar/gkv1507.
Coombes, K. N., S.; Joy, C.; Hu, J.; Baggerly, K.; et al. (2011). SuperCurve Package. R packageversion 1.4.1.
Ding, Z., Mangino, M., Aviv, A., Spector, T., Durbin, R., and Consortium, U.K. (2014). Estimating telomere length from whole genome sequence data. Nucleic acids research 42, e75.
Eckel-Passow, J. E., Lachance, D. H., Molinaro, A. M., Walsh, K. M., Decker, P. A., Sicotte, H., Pekmezci, M., Rice, T., Kosel, M. L., Smirnov, I. V. et al. (2015) Glioma Groups Based on 1p/19q, IDH, and TERT Promoter Mutations in Tumors. N. Engl. J. Med.; 372: 2499-2508
Faith, J. J., Hayete, B., Thaden, J. T., Mogno, I., Wierzbowski, J., Cottarel, G., Kasif, S., Collins, J. J., and Gardner, T. S. (2007). Large-scale mapping and validation of Escherichia coli transcriptional regulation from a compendium of expression profiles. Plos Biol 5, 54-66.
Frattini, V., Trifonov, V., Chan, J. M., Castano, A., Lia, M., Abate, F., Keir, S. T., Ji, A. X., Zoppoli, P., Niola, F., et al. (2013). The integrated landscape of driver genomic alterations inglioblastoma. Nature genetics 45, 1141-1149.
Flynn, R. L., Cox, K. E., Jeitany, M., Wakimoto, H., Bryll, A. R., Ganem, N. J., Bersani, F., Pineda, J. R., Suva, M. L., Benes, C. H. et al. (2015) Alternative lengthening of telomeres renders cancer cells hypersensitive to ATR inhibitors. Science; 347: 273-277
Gardiner-Garden, M., and Frommer, M. (1987). CpG islands in vertebrate genomes. J Mol Biol 196, 261-282.
Gleize, V., Alentorn, A., Connen de Kerillis, L., Labussiere, M., Nadaradjane, A., Mundwiller, E., Ottolenghi, C., Mangesius, S., Rahimian, A., Ducray, F., et al. (2015). CIC inactivating mutations identify aggressive subset of 1p19q codeleted gliomas. Annals of neurology.
Golbeck, J., and Mutton, P. (2005). Spring-Embedded graphs for semantic visualization. Visualizing the Semantic Web, 172-182.
Gonzalez-Angulo, A. M., Hennessy, B. T., Meric-Bernstam, F., Sahin, A., Liu, W., Ju, Z., Carey, M. S., Myhre, S., Speers, C., Deng, L., et al. (2011). Functional proteomics can define prognosis and predict pathologic complete response in patients with breast cancer. Clin Proteomics 8, 11.
Graham, V., Khudyakov, J., Ellis, P., and Pevny, L. (2003) SOX2 functions to maintain neural progenitor identity. Neuron.; 39: 749-765
Guintivano, J., Aryee, M. J., and Kaminsky, Z. A. (2013). A cell epigenotype specific model for the correction of brain cellular heterogeneity bias and its application to age, brain region andmaj or depression. Epigenetics 8, 290-302.
Harrell, F. E., Jr., Califf, R. M., Pryor, D. B., Lee, K. L., and Rosati, R. A. (1982). Evaluating the yield of medical tests. JAMA 247, 2543-2546.
Heinz, S., Benner, C., Spann, N., Bertolino, E., Lin, Y. C., Laslo, P., Cheng, J. X., Murre, C., Singh, H., and Glass, C. K. (2010). Simple combinations of lineage-determining transcription factors prime cis-regulatory elements required for macrophage and B cell identities. Mol Cell 38, 576-589.
Hennessy, B. T., Lu, Y., Gonzalez-Angulo, A. M., Carey, M. S., Myhre, S., Ju, Z., Davies, M. A., Liu, W., Coombes, K., Meric-Bernstam, F., et al. (2010). A Technical Assessment of the Utility of Reverse Phase Protein Arrays for the Study of the Functional Proteome in Non-microdissected Human Breast Cancers. Clin Proteomics 6, 129-151.
Hennessy, B. T., Lu, Y. L., Poradosu, E., Yu, Q. H., Yu, S. X., Hall, H., Carey, M. S., Ravoori, M., Gonzalez-Angulo, A. M., Birch, R., et al. (2007). Pharmacodynamic markers of perifosine efficacy. Clin Cancer Res 13, 7421-7431.
Holland, E. C., Celestino, J., Dai, C., Schaefer, L., Sawaya, R. E., and Fuller, G. N. (2000) Combined activation of Ras and Akt in neural progenitors induces glioblastoma formation in mice. Nat. Genet.; 25: 55-57
Horn, S., Figl, A., Rachakonda, P. S., Fischer, C., Sucker, A., Gast, A., Kadel, S., Moll, I., Nagore, E., Hemminki, K. et al. (2013) TERT promoter mutations in familial and sporadic melanoma. Science; 339: 959-961
Hu, J., He, X., Baggerly, K. A., Coombes, K. R., Hennessy, B. T., and Mills, G. B. (2007). Nonparametric quantification of protein lysate arrays. Bioinformatics 23, 1986-1994.
Huang, D. W., Sherman, B. T., and Lempicki, R. A. (2009). Systematic and integrative analysis of large gene lists using DAVID bioinformatics resources. Nat Protoc 4, 44-57.
Hung, N., Chen, Y. J., Taha, A., Olivecrona, M., Boet, R., Wiles, A., Warr, T., Shaw, A., Eiholzer, R., Baguley, B. C., et al. (2014). Increased paired box transcription factor 8 has a survival function in glioma. BMC cancer 14, 159.
Imielinski, M., Berger, A. H., Hammerman, P. S., Hernandez, B., Pugh, T. J., Hodis, E., Cho, J., Suh, J., Capelletti, M., Sivachenko, A., et al. (2012). Mapping the hallmarks of lung adenocarcinoma with massively parallel sequencing. Cell 150, 1107-1120.
Johnson, W. E., Li, C., and Rabinovic, A. (2007). Adjusting batch effects in microarray expression data using empirical Bayes methods. Biostatistics 8, 118-127.
Jones, D. T., Hutter, B., Jager, N., Korshunov, A., Kool, M., Warnatz, H. J., Zichner, T., Lambert, S. R., Ryzhova, M., Quang, D. A. . . . and International Cancer Genome Consortium PedBrain Tumor Project. (2013) Recurrent somatic alterations of FGFR1 and NTRK2 in pilocytic astrocytoma. Nat. Genet.; 45: 927-932
Kent, W. J. (2002). BLAT—the BLAST-like alignment tool. Genome Res 12, 656-664.
Kent, W. J., Sugnet, C. W., Furey, T. S., Roskin, K. M., Pringle, T. H., Zahler, A. M., and Haussler, D. (2002). The human genome browser at UCSC. Genome Res 12, 996-1006.
Killela, P. J., Reitman, Z. J., Jiao, Y., Bettegowda, C., Agrawal, N., Diaz, L. A. Jr., Friedman, A. H., Friedman, H., Gallia, G. L., Giovanella, B. C. et al. (2013) TERT promoter mutations occur frequently in gliomas and a subset of tumors derived from cells with low rates of self-renewal. Proc. Natl. Acad. Sci. USA.; 110: 6021-6026
Kim, H., Zheng, S., Amini, S. S., Virk, S. M., Mikkelsen, T., Brat, D. J., Grimsby, J., Sougnez, C., Muller, F., Hu, J. et al. (2015) Whole-genome and multisector exome sequencing of primary and post-treatment glioblastoma reveals patterns of tumor evolution. Genome Res.; 25: 316-327
Koboldt, D. C., Zhang, Q., Larson, D. E., Shen, D., McLellan, M. D., Lin, L., Miller, C. A., Mardis, E. R., Ding, L., and Wilson, R. K. (2012). VarScan 2: somatic mutation and copy number alteration discovery in cancer by exome sequencing. Genome research 22, 568-576.
Kon, A., Shih, L. Y., Minamino, M., Sanada, M., Shiraishi, Y., Nagata, Y., Yoshida, K., Okuno, Y., Bando, M., Nakato, R. et al. (2013) Recurrent mutations in multiple components of the cohesin complex in myeloid neoplasms. Nat. Genet.; 45: 1232-1237
Kurscheid, S., Bady, P., Sciuscio, D., Samarzija, I., Shay, T., Vassallo, I., Criekinge, W. V., Daniel, R. T., van den Bent, M. J., Marosi, C. et al. (2015)
Chromosome 7 gain and DNA hypermethylation at the HOXA10 locus are associated with expression of a stem cell related HOX-signature in glioblastoma. Genome Biol.; 16: 16
Lambert, S. R., Witt, H., Hovestadt, V., Zucknick, M., Kool, M., Pearson, D. M., Korshunov, A., Ryzhova, M., Ichimura, K., Jabado, N., et al. (2013). Differential expression and methylation of brain developmental genes define location-specific subsets of pilocytic astrocytoma. Acta neuropathologica 126, 291-301.
Lawrence, M., Huber, W., Pages, H., Aboyoun, P., Carlson, M., Gentleman, R., Morgan, M. T., and Carey, V. J. (2013a). Software for computing and annotating genomic ranges. PLoS Comput Biol 9, e1003118.
Lawrence, M. S., Stojanov, P., Polak, P., Kryukov, G. V., Cibulskis, K., Sivachenko, A., Carter, S. L., Stewart, C., Mermel, C. H., Roberts, S. A., et al. (2013b). Mutational heterogeneity in cancer and the search for new cancer-associated genes. Nature 499, 214-218.
Liang, J., Shao, S. H., Xu, Z. X., Hennessy, B., Ding, Z., Larrea, M., Kondo, S., Dumont, D. J., Gutterman, J. U., Walker, C. L., et al. (2007). The energy sensing LKB1-AMPK pathwayregulates p27(kip1) phosphorylation mediating the decision to enter autophagy or apoptosis. Nat Cell Biol 9, 218-224.
Lodato, M. A., Ng, C. W., Wamstad, J. A., Cheng, A. W., Thai, K. K., Fraenkel, E., Jaenisch, R., and Boyer, L. A. (2013). SOX2 co-occupies distal enhancer elements with distinct POU factors in ESCs and NPCs to specify cell state. PLoS Genet.; 9: e1003288
Louis, D. N., Ohgaki, H., Wiestler, O. D., Cavenee, W. K., Burger, P. C., Jouvet, A., Scheithauer, B. W., and Kleihues, P. (2007) The 2007 WHO classification of tumours of the central nervous system. Acta Neuropathol. 2007; 114: 97-109
Mazor, T., Pankov, A., Johnson, B. E., Hong, C., Hamilton, E. G., Bell, R. J., Smirnov, I. V., Reis, G. F., Phillips, J. J., Barnes, M. J. et al. (2015) DNA Methylation and Somatic Mutations Converge on the Cell Cycle and Define Similar Evolutionary Histories in Brain Tumors. Cancer Cell.; 28: 307-317
McCarroll, S. A., Kuruvilla, F. G., Korn, J. M., Cawley, S., Nemesh, J., Wysoker, A., Shapero, M. H., de Bakker, P. I., Maller, J. B., Kirby, A., et al. (2008). Integrated detection and populationgenetic analysis of SNPs and copy number variation. Nature genetics 40, 1166-1174.
McPherson, A., Hormozdiari, F., Zayed, A., Giuliany, R., Ha, G., Sun, M. G., Griffith, M., Heravi Moussavi, A., Senz, J., Melnyk, N., et al. (2011). deFuse: an algorithm for gene fusion discovery in tumor RNA-Seq data. PLoS computational biology 7, e1001138.
Mermel, C. H., Schumacher, S. E., Hill, B., Meyerson, M. L., Beroukhim, R., and Getz, G. (2011). GISTIC2.0 facilitates sensitive and confident localization of the targets of focal somatic copynumber alteration in human cancers. Genome biology 12, R41.
Mur, P., Mollejo, M., Ruano, Y., de Lope, A. R., Fiano, C., Garcia, J. F., Castresana, J. S., Hernandez-Lain, A., Rey, J. A., and Melendez, B. (2013). Codeletion of 1p and 19q determines distinct gene methylation and expression profiles in IDH-mutated oligodendroglial tumors. Acta neuropathologica 126, 277-289.
Neeley, E. S., Kornblau, S. M., Coombes, K. R., and Baggerly, K. A. (2009). Variable slope normalization of reverse phase protein arrays. Bioinformatics 25, 1384-1389.
Noushmehr, H., Weisenberger, D. J., Diefes, K., Phillips, H. S., Pujara, K., Berman, B. P., Pan, F., Pelloski, C. E., Sulman, E. P., Bhat, K. P. . . . , and Cancer Genome Atlas Research Network. (2010) Identification of a CpG island methylator phenotype that defines a distinct subgroup of glioma. Cancer Cell.; 17: 510-522
Olar, A., Wani, K. M., Alfaro-Munoz, K. D., Heathcock, L. E., van Thuijl, H. F., Gilbert, M. R., Armstrong, T. S., Sulman, E. P., Cahill, D. P., Vera-Bolanos, E. et al. (2015) IDH mutation status and role of WHO grade and mitotic index in overall survival in grade II-III diffuse gliomas. Acta Neuropathol. 2015; 129: 585-596.
Omberg, L., et al. (2013), Enabling transparent and collaborative computational analysis of 12 tumor types within The Cancer Genome Atlas. Nat Genet 45(10): p. 1121-6.
Ozawa, T., Riester, M., Cheng, Y. K., Huse, J. T., Squatrito, M., Helmy, K., Charles, N., Michor, F., and Holland, E. C. (2014) Most human non-GCIMP glioblastoma subtypes evolve from a common proneural-like precursor glioma. Cancer Cell.; 26: 288-300.
Peters, J. M. and Nishiyama, T. (2012) Sister chromatid cohesion. Cold Spring Harb. Perspect. Biol.; 4: a011130
Pineda, C. T., Ramanathan, S., Fon Tacer, K., Weon, J. L., Potts, M. B., Ou, Y. H., White, M. A., and Potts, P. R. (2015) Degradation of AMPK by a cancer-specific ubiquitin ligase. Cell.; 160: 715-728
Radenbaugh, A. J., Ma, S., Ewing, A., Stuart, J. M., Collisson, E. A., Zhu, J., and Haussler, D(2014). RADIA: RNA and DNA integrated analysis for somatic mutation detection. PloS one 9, e111516.
Ramos, A. H., Lichtenstein, L., Gupta, M., Lawrence, M. S., Pugh, T. J., Saksena, G., Meyerson, M., and Getz, G. (2015). Oncotator: cancer variant annotation tool. Hum Mutat 36, E2423-2429.
Reuss, D. E., Mamatj an, Y., Schrimpf, D., Capper, D., Hovestadt, V., Kratz, A., Sahm, F., Koelsche, C., Korshunov, A., Olar, A. et al. (2015) IDH mutant diffuse and anaplastic astrocytomas have similar age at presentation and little difference in survival: a grading problem for WHO. Acta Neuropathol.; 129: 867-873.
Robinson, M. D., McCarthy, D. J., and Smyth, G. K. (2010). edgeR: a Bioconductor package for differential expression analysis of digital gene expression data. Bioinformatics 26, 139-140.
Roggendorf, W., Strupp, S., and Paulus, W. (1996) Distribution and characterization of microglia/macrophages in human brain tumors. Acta Neuropathol.; 92: 288-293
Schwartzbaum, J. A., Fisher, J. L., Aldape, K. D., and Wrensch, M. (2006) Epidemiology and molecular pathology of glioma. Nat. Clin. Pract. Neurol.; 2: 494-503.
Sethi, G., et al. (2012), An RNA interference lethality screen of the human druggable genome to identify molecular vulnerabilities in epithelial ovarian cancer. PLoS One, 7(10): p. e47086.
Smyth, G. K. (2005). Limma: linear models for microarray data. Bioinformatics and computational biology solutions using R and Bioconductor, 397-420.
Solomon, D. A., Kim, T., Diaz-Martinez, L. A., Fair, J., Elkahloun, A. G., Harris, B. T., Toretsky, J. A., Rosenberg, S. A., Shukla, N., Ladanyi, M. et al. (2011) Mutational inactivation of STAG2 causes aneuploidy in human cancer. Science; 333: 1039-1043
Spearman, C. (1904). The proof and measurement of association between two things. Am J Psychol 15, 72-101.
Sturm, D., Bender, S., Jones, D. T., Lichter, P., Grill, J., Becher, O., Hawkins, C., Majewski, J., Jones, C., Costello, J. F. et al. (2014) Paediatric and adult glioblastoma: multiform (epi)genomic culprits emerge. Nat. Rev. Cancer; 14: 92-107
Sturm, D., Witt, H., Hovestadt, V., Khuong-Quang, D. A., Jones, D. T., Konermann, C., Pfaff, E., Tonjes, M., Sill, M., Bender, S., et al. (2012). Hotspot mutations in H3F3A and IDH1 define distinct epigenetic and biological subgroups of glioblastoma. Cancer cell 22, 425-437.
Subramanian, A., Tamayo, P., Mootha, V. K., Mukherjee, S., Ebert, B. L., Gillette, M. A., Paulovich, A., Pomeroy, S. L., Golub, T. R., Lander, E. S., et al. (2005). Gene set enrichment analysis: a knowledge-based approach for interpreting genome-wide expression profiles. Proc Natl Acad Sci USA 102, 15545-15550.
Suvà, M. L., Rheinbay, E., Gillespie, S. M., Patel, A. P., Wakimoto, H., Rabkin, S. D., Riggi, N., Chi, A. S., Cahill, D. P., Nahed, B. V. et al. (2014) Reconstructing and reprogramming the tumor-propagating potential of glioblastoma stem-like cells. Cell; 157: 580-594
Suzuki, H., Aoki, K., Chiba, K., Sato, Y., Shiozawa, Y., Shiraishi, Y., Shimamura, T., Niida, A., Motomura, K., Ohka, F. et al. (2015) Mutational landscape and clonal architecture in grade II and III gliomas. Nat. Genet.; 47: 458-468
Takai, D., and Jones, P. A. (2002). Comprehensive analysis of CpG islands in humanchromosomes 21 and 22. Proc Natl Acad Sci USA 99, 3740-3745.
TCGA_Network (2015). Comprehensive, Integrative Genomic Analysis of Diffuse Lower Grade Gliomas. New England Journal of Medicine.
Therneau, T. M. (2014). A package for survival analysis in S. version 2.37-7. http://CRANRprojectorg/package=survival.
Therneau, T. M., and Grambsch, P. M. (2000). Modeling survival data: extending the Cox model (New York: Springer).
Tibes, R., Qiu, Y., Lu, Y., Hennessy, B., Andreeff, M., Mills, G. B., and Kornblau, S. M. (2006). Reverse phase protein array: validation of a novel proteomic technology and utility for analysis of primary leukemia specimens and hematopoietic stem cells. Mol Cancer Ther 5, 2512-2521.
Torres-Garcia, W., Zheng, S., Sivachenko, A., Vegesna, R., Wang, Q., Yao, R., Berger, M. F., Weinstein, J. N., Getz, G., and Verhaak, R. G. (2014). PRADA: pipeline for RNA sequencing data analysis. Bioinformatics 30, 2224-2226.
Trifonov, V., Pasqualucci, L., Dalla Favera, R., and Rabadan, R. (2013). MutComFocal: an integrative approach to identifying recurrent and focal genomic alterations in tumor samples. BMC Syst Biol 7, 25.
Turcan, S., Rohle, D., Goenka, A., Walsh, L. A., Fang, F., Yilmaz, E., Campos, C., Fabius, A. W., Lu, C., Ward, P. S., et al. (2012). IDH1 mutation is sufficient to establish the glioma hypermethylator phenotype. Nature 483, 479-483. van den Bent, M. J. (2010) Interobserver variation of the histopathological diagnosis in clinical trials on glioma: a clinician's perspective. Acta Neuropathol. 2010; 120: 297-304
Vaske, C. J., Benz, S. C., Sanborn, J. Z., Earl, D., Szeto, C., Zhu, J. C., Haussler, D., and Stuart, J. M. (2010). Inference of patient-specific pathway activities from multi-dimensional cancer genomics data using PARADIGM. Bioinformatics 26, i237-i245.
Verhaak, R. G., Hoadley, K. A., Purdom, E., Wang, V., Qi, Y., Wilkerson,
M. D., Miller, C. R., Ding, L., Golub, T., Mesirov, J. P., et al. (2010). Integrated genomic analysis identifies clinically relevant subtypes of glioblastoma characterized by abnormalities in PDGFRA, IDH1, EGFR, and NFL Cancer cell 17, 98-110.
Yan, H., Parsons, D. W., Jin, G., McLendon, R., Rasheed, B. A., Yuan, W., Kos, I., Batinic-Haberle, I., Jones, S., Riggins, G. J. et al. (2009) IDH1 and IDH2 mutations in gliomas. N. Engl. J. Med.; 360: 765-773
Yoshihara, K., Wang, Q., Tones-Garcia, W., Zheng, S., Vegesna, R., Kim, H., and Verhaak, R. G. (2014). The landscape and therapeutic relevance of cancer-associated transcript fusions. Oncogene.
Yoshihara, K., Shahmoradgoli, M., Martinez, E., Vegesna, R., Kim, H., Torres-Garcia, W., Treviño, V., Shen, H., Laird, P. W., Levine, D. A. et al. Inferring tumour purity and stromal and immune cell admixture from expression data.

1. A method of classifying a glioma in a patient, the method comprising:
identifying with respect to the glioma:
isocitrate dehydrogenase genes (IDH) mutation status;
DNA methylation cluster;
RNA cluster;
telomere length;
telomere maintenance; and
at least one biomarker; and
based in the identifications, classifying the glioma as IDH mutant/G-CIMP low glioma type, IDH mutant/G-CIMP high glioma type, IDH mutant/Codel glioma type, DH wild type/Classic like glioma type, IDH wild type/Mesenchymal-like glioma type, IDH wild type/LGm6-GBM glioma type, or PA-like glioma type.

2. The method of claim 1, wherein IDH mutation status is based on a mutation in and IDH1 or IDH2 gene, or the presence of a wild type version of both genes.

3. The method of claim 1, wherein the DNA methylation cluster is LGm1, LGm2, LGm3, LGm4, LGm5, or LGm6.

4. The method of claim 1, wherein the RNA cluster is LGr1/2, LGr3, or LGr4.

5. The method of claim 1, wherein telomere length is identified as elongated, shortened, or stable.

6. The method of claim 1, wherein the telomere maintenance is identified based on the presence of mutant to wild type Alpha-thalassemia X-linked (ATRX).

7. The method of claim 1, wherein the teolmere maintenance is identified based on the upregulation or normal expression of telomerase reverse transcriptase (TERT).

8. The method of claim 1, wherein the biomarker comprises upregulation of Epidermal Growth Factor Receptor (EGFR).

9. The method of claim 1, wherein the biomarker comprises a mutation in Tumor protein p53.

10. The method of claim 1, wherein the biomarker comprises an IDH mutant-codel.

11. The method of claim 1, wherein the biomarker comprises chromosome 7 (chr7) amplification coupled with a chromosome 10 (chr10) deletion.

12. The method of claim 1, wherein the biomarker comprises a Cyclin-dependent kinase 4 (CDK4) amplification coupled with a Cyclin Dependent Kinase Inhibitor 2A (CDKN2A) deletion.

13. The method of claim 1, wherein the biomarker comprises a chromosome 19 (chr19) amplification coupled with a chromosome 20 (chr20) amplification.

14. The method of claim 1, wherein the biomarker comprises a B-Raf gene (BRAF) mutation coupled with a Neurofibromin 1 (NF1) mutation.

15. The method of claim 1, wherein the IDH mutant/G-CIMP low glioma type exhibits an IDH mutation, the LGm1 DNA methylation cluster, the LGr3, RNA cluster, elongated telomere length, an ATRX mutation, a TP53 mutation, and a CDK4 amplification coupled with a CDKN2A deletion.

16. The method of claim 1, wherein the IDH mutant/G-CIMP high glioma type exhibits an IDH mutation, the LGm2 DNA methylation cluster, the LGr3 RNA cluster, elongated telomere length, an ATRX mutation, and a TP53 mutation.

17. The method of claim 1, wherein the IDH mutant/Codel glioma type exhibits an IDH mutation, the LGm3 DNA methylation cluster, the LGr1/2 RNA cluster, shortened telomere length, upregulation of TERT, and an IDH mutant-codel.

18. The method of claim 1, wherein the IDH wild type/Classic like glioma type exhibits wild type IDH, the LGm4 DNA methylation cluster, the LGr4 RNA cluster, shortened telomere length, upregulation of TERT, amplified EGFR, amplified chr 7 coupled with a chr 10 deletion, and amplified chr 19 coupled with amplified chr 20.

19. The method of claim 1, wherein the IDH wild type/Mesenchymal-like glioma type exhibits wild type IDH, the LGm5 DNA methylatin cluster, the LGr4 RNA cluster, shortened telomere length, amplified EGFR, and amplified chr 7 coupled with a chr 10 deletion.

20. The method of claim 1, wherein the IDH wild type/LGm6-GBM glioma type exhibits wild type IDH, the LGm6 DNA methylation cluster, the LGr4 RNA cluster, stable telomer length, amplified EGFR, a CDK4 amplification coupled with a CDKN2A deletion, and a BRAF mutation coupled with a NF1 mutation.

21. The method of claim 1, wherein the PA-like glioma type exhibits wild type IDH, the LGm6 DNA methylation cluster, the LGr4 RNA cluster, stable telomer length, and a BRAF mutation coupled with a NF1 mutation.
